(19) United States
(12) Patent Application Publication
(10) Pub. No.: US 2007/0021624 A1
(43) Pub. Date: Jan. 25, 2007

(54) Steroid sulphatase inhibitors

(76) Inventors: Michael John Reed, Slough (GB); Barry Victor Lloyd Potter, Slough (GB)

Correspondence Address: FROMMER LAWRENCE & HAUG, 745 FIFTH AVENUE- 10TH FL., NEW YORK, NY, 10151, US

(21) Appl. No.: 11/406,079

(22) Filed: Apr. 18, 2006

Related U.S. Application Data
(63) Continuation-in-part of application No. 10/084,235, filed on Feb. 25, 2002, now Pat. No. 7,098,199.
Application No. 10/084,235 is a division of application No. 09/579,163, filed on May 25, 2000, now Pat. No. 6,642,397.
Application No. 09/579,163 is a division of application No. 09/238,345, filed on Jan. 27, 1999, now Pat. No. 6,187,766.
Application No. 09/238,345 is a division of application No. 09/111,927, filed on Jul. 8, 1998, now Pat. No. 6,011,024.
Application No. 09/111,927 is a continuation-in-part of application No. 08/458,352, filed on Jun. 2, 1995, now Pat. No. 5,830,886.
Application No. 08/458,352 is a division of application No. 08/196,192, filed on Dec. 27, 1994, now Pat. No. 5,616,574.
Application No. 09/111,927 is a continuation-in-part of application No. PCT/GB97/00600, filed on Mar. 4, 1997.

(30) Foreign Application Priority Data
Aug. 29, 1991 (GB) 9118478.8
Mar. 5, 1996 (GB) 9604709.7
Mar. 19, 1996 (GB) 9605725.2
Feb. 16, 1996 (GB) 9603325.3
Dec. 5, 1996 (GB) 9625334.9

(51) Int. Cl. C07J 1/00

(52) U.S. Cl. 552/626

(57) ABSTRACT

A method of inhibiting steroid sulphatase activity in a subject in need of same is described.
The method comprises administering to said subject a steroid sulphatase inhibiting amount of a ring system compound; which ring system compound comprises a ring to which is attached a sulphamate group of the formula 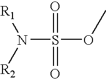
wherein each of R₁ and R₂ is independently selected from H, alkyl, alkenyl, cycloalkyl and aryl, or together represent alkylene optionally containing one or more hetero atoms or groups in the alkylene chain; and wherein said compound is an inhibitor of an enzyme having steroid sulphatase activity (E.C.3.1.6.2); and if the sulphamate group of said compound is replaced with a sulphate group to form a sulphate compound and incubated with a steroid sulphatase enzyme (E.C.3.1.6.2) at a pH 7.4 and 37° C. it would provide a Kₘ value of less than 50 μM.

### REFERENCE TO RELATED APPLICATIONS

This application is a continuation-in-part of U.S. application Ser. No. 10/084,235 filed Feb. 25, 2002 as a division of U.S. application Ser. No. 09/579,163, filed May 25, 2000, which in turn was a division of U.S. application Ser. No. 09/238,345, filed Jan. 27, 1999, which in turn was a division of U.S. application Ser. No. 09/111,927, filed Jul. 8, 1998, incorporated herein by reference and now U.S. Pat. No. 6,011,024, which in turn was a continuation-in-part of U.S. application Ser. No. 08/458,352, filed Jun. 2, 1995, now U.S. Pat. No. 5,830,886, which was a division of U.S. application Ser. No. 08/196,192, filed (§102(e) date of) Dec. 27, 1994, now U.S. Pat. No. 5,616,574. U.S. application Ser. No. 08/196,192 was the U.S. National Phase of PCT/GB92/01587, filed Aug. 28, 1992 and designating the U.S, and incorporated herein by reference. U.S. application Ser. No. 08/196,192 has a §371 date of Dec. 27, 1994 and a §102(e) date of Dec. 27, 1994. PCT/GB92/01587 was published as WO93/05064, has a publication date of Mar. 18, 1993, and claims priority from United Kingdom patent application No. 9118478, filed Aug. 29, 1991. U.S. Ser. No. 09/111,927 was also a continuation-in-part of PCT patent application number PCT/GB97/00600, filed Mar. 4, 1997, designating the U.S., and claiming priority from United Kingdom patent applications 9604709.7 and 9605725.2, filed Mar. 5 and 19, 1996, respectively. PCT/GB97/00600 was published as WO 97/32872 on Sep. 12, 1997. U.S. Ser. No. 09/111,927 was also a continuation-in-part of PCT patent application number PCT/GB97/00444, filed Feb. 17, 1997, designating the U.S., and claiming priority from United Kingdom patent application 9603325.3, filed Feb. 16, 1996. PCT/GB97/00444 was published as WO 97/30041 on Aug. 21, 1997. U.S. Ser. No. 09/111,927 was also a continuation-in-part of PCT patent application number PCT/GB97/03352, filed Dec. 4, 1997, designating the U.S., and claiming priority from United Kingdom patent application 9625334.9, filed Dec. 5, 1996. PCT/GB97/03352 was published as WO 98/24802 on Jun. 11, 1998. Reference is also made to EP 1 568 381 published Aug. 31, 2005 from EP Application 20030754063 filed Oct. 9, 2003 and corresponding US publication 2006/0035875, published Feb. 16, 2006 from U.S. application Ser. No. 10/531,099 filed Apr. 7, 2005. Each of PCT/GB97/00600 (WO 97/32872), PCT/GB97/00444 (WO 97/30041), PCT/GB97/03352 (WO 98/24802), EP 1 568 381, and U.S. Ser. No. 10/531,099 is hereby incorporated herein by reference. In addition, all of the above-mentioned applications, as well as all documents cited herein and documents referenced or cited in documents cited herein, are hereby incorporated herein by reference.

### TECHNICAL FIELD

The present invention relates to a therapeutic agent for a hormone-dependent cancer, comprising a steroid-sulfatase inhibitor.

### BACKGROUND ART

Among cancers, there are those wherein proliferation thereof is promoted by hormone(s) (hormone-dependent cancers). Such hormone-dependent cancers include breast cancer, ovarian cancer, endometrial cancer, prostatic cancer, and thyroid cancer.

Nowadays, the hormone-dependent cancers are treated by surgical removal of an organ that secretes a particular hormone (e.g. surgical removal of the ovary), by the administration of an inhibitor that reduces hormone activities in order to suppress the proliferation of the hormone-dependent cancer cells (e.g. hormone therapy and chemotherapy), or the like. In some cases, these therapies may be performed in combination.

Examples of the agents for hormone therapy include antiestrogen agents, aromatase inhibitors, antiandrogen agents, preparations comprising progesterone, and preparations comprising an luteinizing hormone-releasing hormone (LH-RH) agonist.

On the other hand, steroid sulfatase is a hydrolase that converts estrone sulfate, i.e. inactive estrogen, to estrone, i.e. active estrogen, and that converts androstenediol sulfate, i.e. inactive androgen, to androstenediol, i.e. active androgen. Thus, steroid sulfatase is involved in the proliferation of mammary gland epithelial cells, hormone-dependent cancer cells or tumor cells.

A high estrogen level in breast cancer is considered to be caused by the hydrolysis of estrone sulfate to estrone by steroid sulfatase (estrone sulfatase). Therefore, steroid-sulfatase inhibitors are considered to be effective therapeutic agents for the treatment of estrogen-dependent breast cancer (a hormone-dependent cancer), and further to be effective for preventing or treating other diseases in which estrones are considered to be involved, e.g. endometrial cancer, ovarian cancer, endometriosis, and adenomyosis uteri. Further, since steroid sulfatase is also involved in the biosynthetic process of androgen, it is considered to be effective for preventing or treating diseases in which androgens are considered to be involved, e.g. prostatic cancer.

It has been reported that estrone-3-sulfamate (EMATE) is a typical inhibitor of steroid sulfatase (See, e.g. U.S. Pat. No. 5,616,574; International Journal of Cancer, 1995, 63: 106-111). However, it has been shown that EMATE is not effective in the treatment of estrone-dependent diseases because of its estrogen-like activity (See, e.g. Cancer Research, 1996, 56: 4950-4955).

So far, a large number of steroid-sulfatase inhibitors have been found (See, e.g. U.S. Pat. No. 5,830,886; WO98/11124; WO98/32763; Expert Opinion on Therapeutic Patents, 1999, 9: 1083).

Such inhibitors include tyramine derivatives (See, e.g. U.S. Pat. No. 5,567,831; Cancer Research, 1997, 57: 702-707; The Journal of Steroid Biochemistry and Molecular Biology, 1996, 59: 41-48; The Journal of Steroid Biochemistry and Molecular Biology, 1999, 68: 31-40; The Journal of Steroid Biochemistry and Molecular Biology, 1999, 69: 227-238), cinnamic acid derivatives (See, e.g. U.S. Pat. No. 6,011,024), and diethylstilbestrol derivatives (See, e.g. The Journal of Steroid Biochemistry and Molecular Biology, 1999, 69: 227-238). Recently, other steroid-sulfatase inhibitors have been disclosed (See, e.g. WO01/04086; WO01/02349).

Furthermore, estrone-3-methylthiophosphonate, estrone-3-methylphosphonate, estrone-3-phenylphosphonothioate, estrone-3-phenylphosphonate (See, e.g. U.S. Pat. No. 5,604,215; Cancer Research, 1993, 53: 298-303; Bioorganic & Medicinal Chemistry Letters, 1993, 3: 313-318), and 3-monoalkylthiophosphate derivatives (See, e.g. WO91/13083) have been disclosed as steroid-sulfatase inhibitors.

In addition to the above, other steroid-sulfatase inhibitors have been disclosed (See, e.g. WO93/05064; WO97/30041; WO99/33858; WO99/52890; WO01/36398; WO00/43408).

### DISCLOSURE OF THE INVENTION

An object of the present invention is to provide a therapeutic agent for a hormone-dependent cancer, which comprises a steroid-sulfatase inhibitor, and an agent for hormone therapy and/or an agent for chemotherapy, and the like.

The present invention relates to the following paragraphs (1) to (36):

(1) A therapeutic agent for a hormone-dependent cancer, which comprises (a) a steroid-sulfatase inhibitor and (b) an agent for hormone therapy and/or an agent for chemotherapy, which may be administered together or separately at an interval.

(2) A method for treating a hormone-dependent cancer, which comprises administering (a) a steroid-sulfatase inhibitor and (b) an agent for hormone therapy and/or an agent for chemotherapy together or separately at an interval.

(3) A steroid-sulfatase inhibitor which is used in combination with an agent for hormone therapy and/or an agent for chemotherapy, and which is administered together therewith or separately therefrom at an interval.

(4) A kit for treating a hormone-dependent cancer, which comprises a first component comprising (a) a steroid-sulfatase inhibitor and a second component comprising (b) an agent for hormone therapy and/or an agent for chemotherapy.

(5) A pharmaceutical composition, which comprises (a) a steroid-sulfatase inhibitor and (b) an agent for hormone therapy and/or an agent for chemotherapy.

(6) Use of (a) a steroid-sulfatase inhibitor and (b) an agent for hormone therapy and/or an agent for chemotherapy for the manufacture of a therapeutic agent for a hormone-dependent cancer.

(7) The therapeutic agent for a hormone-dependent cancer according to (1), wherein the steroid-sulfatase inhibitor is a composition comprising, as an active ingredient, a compound represented by Formula (I) or a pharmaceutically acceptable salt thereof: 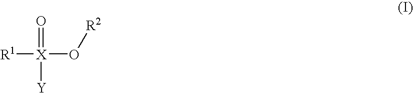
[wherein X represents a phosphorus atom or a sulfur atom, and when X is a phosphorus atom, Y is hydroxy, and when X is a sulfur atom, Y is oxo; R¹ represents a hydrogen atom, substituted or unsubstituted lower alkyl, substituted or unsubstituted cycloalkyl, substituted or unsubstituted lower alkenyl, substituted or unsubstituted aryl, or —NR³R⁴ (wherein R³ and R⁴ may be the same or different and each represents a hydrogen atom, substituted or unsubstituted lower alkyl, substituted or unsubstituted cycloalkyl, substituted or unsubstituted lower alkenyl, or substituted or unsubstituted aryl, or R³ and R⁴ are combined together with the adjacent nitrogen atom thereto to form a substituted or unsubstituted heterocyclic group); and —O—R² represents a monocyclic alcohol residue or a polycyclic alcohol residue].

(8) The method for treating a hormone-dependent cancer according to (2), wherein the steroid-sulfatase inhibitor is a composition comprising, as an active ingredient, the compound represented by Formula (I) described in (7) or a pharmaceutically acceptable salt thereof.

(9) The steroid-sulfatase inhibitor according to (3), wherein the steroid-sulfatase inhibitor is a composition comprising, as an active ingredient, the compound represented by Formula (I) described in (7) or a pharmaceutically acceptable salt thereof.

(10) The kit for treating according to (4), wherein the steroid-sulfatase inhibitor is a composition comprising, as an active ingredient, the compound represented by Formula (I) described in (7) or a pharmaceutically acceptable salt thereof.

(11) The pharmaceutical composition according to (5), wherein the steroid-sulfatase inhibitor is a composition comprising, as an active ingredient, the compound represented by Formula (I) described in (7) or a pharmaceutically acceptable salt thereof.

(12) The use of (a) a steroid-sulfatase inhibitor and (b) an agent for hormone therapy and/or an agent for chemotherapy according to (6), wherein the steroid-sulfatase inhibitor is a composition comprising, as an active ingredient, the compound represented by Formula (I) described in (7) or a pharmaceutically acceptable salt thereof.

(13) The therapeutic agent for a hormone-dependent cancer according to (1), wherein the steroid-sulfatase inhibitor is a composition comprising, as an active ingredient, a compound represented by Formula (IA) or a pharmaceutically acceptable salt thereof: 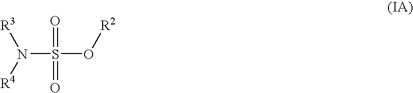
(wherein —O—R², R³, and R⁴ have the same meanings as defined above, respectively).

(14) The method for treating a hormone-dependent cancer according to (2), wherein the steroid-sulfatase inhibitor is a composition comprising, as an active ingredient, the compound represented by Formula (IA) described in (13) or a pharmaceutically acceptable salt thereof.

(15) The steroid-sulfatase inhibitor according to (3), wherein the steroid-sulfatase inhibitor is a composition comprising, as an active ingredient, the compound represented by Formula (IA) described in (13) or a pharmaceutically acceptable salt thereof.

(16) The kit for treating according to (4), wherein the steroid-sulfatase inhibitor is a composition comprising, as an active ingredient, the compound represented by Formula (IA) described in (13) or a pharmaceutically acceptable salt thereof.

(17) The pharmaceutical composition according to (5), wherein the steroid-sulfatase inhibitor is a composition comprising, as an active ingredient, the compound represented by Formula (IA) described in (13) or a pharmaceutically acceptable salt thereof.

(18) The use of (a) a steroid-sulfatase inhibitor and (b) an agent for hormone therapy and/or an agent for chemotherapy according to (6), wherein the steroid-sulfatase inhibitor is a composition comprising, as an active ingredient, the compound represented by Formula (IA) described in (13) or a pharmaceutically acceptable salt thereof.

(19) The therapeutic agent for a hormone-dependent cancer according to (1), wherein the steroid-sulfatase inhibitor is a composition comprising, as an active ingredient, a compound represented by Formula (IB) or a pharmaceutically acceptable salt thereof: 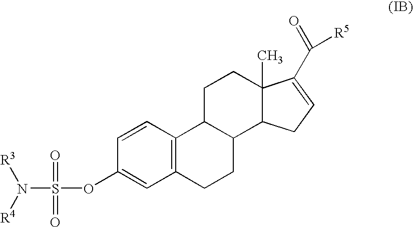
[wherein R³ and R⁴ have the same meanings as defined above, respectively; R⁵ represents a hydrogen atom, substituted or unsubstituted lower alkyl, substituted or unsubstituted cycloalkyl, substituted or unsubstituted lower alkenyl, substituted or unsubstituted lower alkynyl, substituted or unsubstituted aryl, a substituted or unsubstituted heterocyclic group, —NR⁶R⁷ (wherein R⁶ and R⁷ may be the same or different and each represents a hydrogen atom, substituted or unsubstituted lower alkyl, substituted or unsubstituted cycloalkyl, substituted or unsubstituted lower alkenyl, substituted or unsubstituted aryl, or a substituted or unsubstituted heterocyclic group), —OR⁸ (wherein R⁸ represents a hydrogen atom, substituted or unsubstituted lower alkyl, substituted or unsubstituted cycloalkyl, substituted or unsubstituted lower alkenyl, substituted or unsubstituted aryl, or a substituted or unsubstituted heterocyclic group), or —SR^{8A} (wherein R^{8A} has the same meaning as R⁸ defined above)].

(20) The method for treating a hormone-dependent cancer according to (2), wherein the steroid-sulfatase inhibitor is a composition comprising, as an active ingredient, the compound represented by Formula (IB) described in (19) or a pharmaceutically acceptable salt thereof.

(21) The steroid-sulfatase inhibitor according to (3), wherein the steroid-sulfatase inhibitor is a composition comprising, as an active ingredient, the compound represented by Formula (IB) described in (19) or a pharmaceutically acceptable salt thereof.

(22) The kit for treating according to (4), wherein the steroid-sulfatase inhibitor is a composition comprising, as an active ingredient, the compound represented by Formula (IB) described in (19) or a pharmaceutically acceptable salt thereof.

(23) The pharmaceutical composition according to (5), wherein the steroid-sulfatase inhibitor is a composition comprising, as an active ingredient, the compound represented by Formula (IB) described in (19) or a pharmaceutically acceptable salt thereof.

(24) The use of (a) a steroid-sulfatase inhibitor and (b) an agent for hormone therapy and/or an agent, for chemotherapy according to (6), wherein the steroid-sulfatase inhibitor is a composition comprising, as an active ingredient, the compound represented by Formula (IB) described in (19) or a pharmaceutically acceptable salt thereof.

(25) The therapeutic agent for a hormone-dependent cancer according to (1), (7), (13), or (19), wherein the agent for hormone therapy is one or more selected from the group consisting of an antiestrogen agent, an aromatase inhibitor, an antiandrogen agent, a preparation comprising progesterone, and a preparation comprising a luteinizing hormone-releasing hormone (LH-RH) agonist.

(26) The method for treating a hormone-dependent cancer according to (2), (8), (14), or (20), wherein the agent for hormone therapy is one or more selected from the group consisting of an antiestrogen agent, an aromatase inhibitor, an antiandrogen agent, a preparation comprising progesterone, and a preparation comprising a LH-RH agonist.

(27) The steroid-sulfatase inhibitor according to (3), (9), (15), or (21), wherein the agent for hormone therapy is one or more selected from the group consisting of an antiestrogen agent, an aromatase inhibitor, an antiandrogen agent, a preparation comprising progesterone, and a preparation comprising a LH-RH agonist.

(28) The kit for treating according to (4), (10), (16), or (22), wherein the agent for hormone therapy is one or more selected from the group consisting of an antiestrogen agent, an aromatase inhibitor, an antiandrogen agent, a preparation comprising progesterone, and a preparation comprising a LH-RH agonist.

(29) The pharmaceutical composition according to (5), (11), (17), or (23), wherein the agent for hormone therapy is one or more selected from the group consisting of an antiestrogen agent, an aromatase inhibitor, an antiandrogen agent, a preparation comprising progesterone, and a preparation comprising a LH-RH agonist.

(30) The use of (a) a steroid-sulfatase inhibitor and (b) an agent for hormone therapy and/or an agent for chemotherapy according to (6), (12), (18), or (24), wherein the agent for hormone therapy is one or more selected from the group consisting of an antiestrogen agent, an aromatase inhibitor, an antiandrogen agent, a preparation comprising progesterone, and a preparation comprising a LH-RH agonist.

(31) The therapeutic agent for a hormone-dependent cancer according to (1), (7), (13), or (19), wherein the agent for hormone therapy is an antiestrogen agent and/or an aromatase inhibitor.

(32) The method for treating a hormone-dependent cancer according to (2), (8), (14), or (20), wherein the agent for hormone therapy is an antiestrogen agent and/or an aromatase inhibitor.

(33) The steroid-sulfatase inhibitor according to (3), (9), (15), or (21), wherein the agent for hormone therapy is an antiestrogen agent and/or an aromatase inhibitor.

(34) The kit for treating according to (4), (10), (16), or (22), wherein the agent for hormone therapy is an antiestrogen agent and/or an aromatase inhibitor.

(35) The pharmaceutical composition according to (5), (11), (17), or (23), wherein the agent for hormone therapy is an antiestrogen agent and/or an aromatase inhibitor.

(36) The use of (a) a steroid-sulfatase inhibitor and (b) an agent for hormone therapy and/or an agent for chemotherapy according to (6), (12), (18), or (24), wherein the agent for hormone therapy is an antiestrogen agent and/or an aromatase inhibitor.

Any hormone-dependent cancer or tumor, in which cancer cells or tumor cells are stimulated to proliferate by a hormone, can be exemplified as the hormone-dependent cancer treated in the present invention. Such hormone-dependent cancers include breast cancer, ovarian cancer, endometrial cancer, prostatic cancer, and thyroid cancer.

Any steroid-sulfatase inhibitor, which can inhibit the steroid sulfatase activity, can be used as the steroid-sulfatase inhibitor. Examples of such steroid-sulfatase inhibitors include a composition comprising, as an active ingredient, a sulfonate ester, a phosphonate ester, a sulfamate, or a thiophosphate of a monocyclic alcohol or a polycyclic alcohol, or the like or a pharmaceutically acceptable salt thereof.

Specifically, the composition which comprises, as an active ingredient, a compound represented by Formula (I) or a pharmaceutically acceptable salt thereof and the like are exemplified: 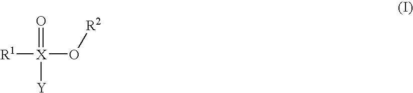
[wherein X represents a phosphorus atom or a sulfur atom, and when X is a phosphorus atom, Y is hydroxy, and when X is a sulfur atom, Y is oxo; R¹ represents a hydrogen atom, substituted or unsubstituted lower alkyl, substituted or unsubstituted cycloalkyl, substituted or unsubstituted lower alkenyl, substituted or unsubstituted aryl, or —NR³R⁴ (wherein R³ and R⁴ may be the same or different and each represents a hydrogen atom, substituted or unsubstituted lower alkyl, substituted or unsubstituted cycloalkyl, substituted or unsubstituted lower alkenyl, or substituted or unsubstituted aryl, or R³ and R⁴ are combined together with an adjacent nitrogen atom thereto to form a substituted or unsubstituted heterocyclic group); and —O—R² represents a monocyclic alcohol residue or a polycyclic alcohol residue].

Among them, the composition which comprises, as an active ingredient, a compound represented by Formula (IA) or a pharmaceutically acceptable salt thereof and the like are preferred:

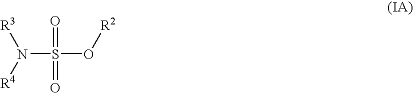

(wherein —O—R², R³, and R⁴ have the same meanings as defined above, respectively). A composition which comprises, as an active ingredient, a compound represented by Formula (IB) or a pharmaceutically acceptable salt thereof and the like are more preferred: 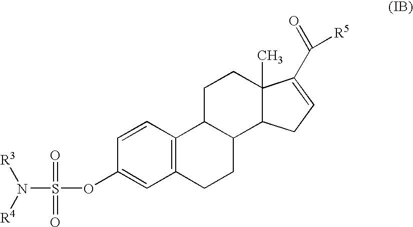
[wherein R³ and R⁴ have the same meanings as defined above, respectively; R5 represents a hydrogen atom, substituted or unsubstituted lower alkyl, substituted or unsubstituted cycloalkyl, substituted or unsubstituted lower alkenyl, substituted or unsubstituted lower alkynyl, substituted or unsubstituted aryl, a substituted or unsubstituted heterocyclic group, —NR⁶R⁷ (wherein R⁶ and R⁷ may be the same or different and each represents a hydrogen atom, substituted or unsubstituted lower alkyl, substituted or unsubstituted cycloalkyl, substituted or unsubstituted lower alkenyl, substituted or unsubstituted aryl, or a substituted or unsubstituted heterocyclic group), —OR⁸ (wherein R⁸ represents a hydrogen atom, substituted or unsubstituted lower alkyl, substituted or unsubstituted cycloalkyl, substituted or unsubstituted lower alkenyl, substituted or unsubstituted aryl, or a substituted or unsubstituted heterocyclic group), or —SR^{8A} (wherein R^{8A} has the same meaning as R⁸ defined above)].

A compound represented by Formula (I) is referred to as Compound (I) hereinafter. Compounds represented by other Formula numbers are also referred to in the same manner.

In the definition of each group in Formulae (I), (IA), and (IB), (i) the monocyclic and polycyclic alcohols constituting monocyclic or polycyclic alcohol residues include any monocyclic and polycyclic alcohol. For example, sulfate compounds (the hydroxyl group is replaced by a sulfate group) corresponding to the alcohols that can be substrates of the steroid sulfatase are preferred. Among them, sulfate compounds having a Km value of less than 50 mu mol/L during incubation at pH 7.4 and 37 DEG C. with an enzyme having a steroid sulfatase activity are more preferred.

Examples of the monocyclic alcohol include a substituted or unsubstituted heterocycle having hydroxy as one of substituents thereof [the heterocycle corresponds to a compound formed by adding one hydrogen atom to a heterocyclic group (x) described later; and substituents other than hydroxy of the substituted heterocycle correspond to substituents of the substituted heterocyclic group (xii) described later], and a substituted or unsubstituted phenol [substituents of the substituted phenol correspond to substituents of substituted heterocyclic group (xii) described later]. Specific examples include tyramine amide derivatives, hydroxycinnamic acid derivatives, and the like.

Examples of the polycyclic alcohol include substituted or unsubstituted fused rings. Examples of the fused ring include di- to penta-cyclic fused rings having 6 to 60 carbon atoms, preferably 6 to 30 carbon atoms and formed by condensing 3- to 8-membered rings having a hydroxyl group as one of the substituents. Each ring may be saturated or unsaturated and may include an element such as a nitrogen atom, an oxygen atom, and a sulfur atom. Specific examples include substituted or unsubstituted sterols; tetrahydronaphthol derivatives; coumarin, chroman, or isoflavone derivatives each having a hydroxyl group as one of substituents; and 4-hydroxytamoxifen derivatives. Substituents of the substituted fused ring and the substituted sterol correspond to substituents of substituted sterol (iii) described later.

(ii) Examples of the sterol include 3-sterol such as estrone, estradiol, estriol, and dehydroepiandrosterone.

(iii) Substituents of the substituted sterol described here may be the same or different. The number of the substituents may be 1 to 3, and examples of the substituents include halogen, nitro, cyano, azide, substituted or unsubstituted lower alkyl, substituted or unsubstituted cycloalkyl, substituted or unsubstituted lower alkenyl, substituted or unsubstituted lower alkynyl, substituted or unsubstituted aryl, substituted or unsubstituted heterocyclic group, —C(═X¹)R⁵ (wherein X¹ represents an oxygen atom or a sulfur atom, R⁵ has the same meaning as defined above), —NR⁹R¹⁰ {wherein R⁹ and R¹⁰ may be the same or different and each represents a hydrogen atom, substituted or unsubstituted lower alkyl, substituted or unsubstituted cycloalkyl, substituted or unsubstituted lower alkenyl, substituted or unsubstituted aryl, a substituted or unsubstituted heterocyclic group, —C(═X²)R¹¹ (wherein X² and R¹¹ have the same meanings as X¹ and R⁵ defined above, respectively), or —SO2R¹² [wherein R¹² represents substituted or unsubstituted lower alkyl, substituted or unsubstituted cycloalkyl, substituted or unsubstituted lower alkenyl, substituted or unsubstituted aryl, a substituted or unsubstituted heterocyclic group, —NR¹³R¹⁴ (wherein R¹³ and R¹⁴ have the same meanings as R⁶ and R⁷ defined above, respectively), or —OR¹⁵ (wherein R¹⁵ has the same meaning as R⁸ defined above)]}, —OR¹⁶ [wherein R¹⁶ represents a hydrogen atom, substituted or unsubstituted lower alkyl, substituted or unsubstituted cycloalkyl, substituted or unsubstituted lower alkenyl, substituted or unsubstituted aryl, a substituted or unsubstituted heterocyclic group, or —SO2R¹⁷ (wherein R¹⁷ has the same meaning as R¹² defined above)], —S(O)mR¹⁸ (wherein m represents 0 or 1, R¹⁸ represents a hydrogen atom, substituted or unsubstituted lower alkyl, substituted or unsubstituted cycloalkyl, substituted or unsubstituted lower alkenyl, substituted or unsubstituted aryl, or a substituted or unsubstituted heterocyclic group), or —SO2R¹⁹ (wherein R¹⁹ has the same meaning as R¹² described above).

The halogen, lower alkyl, cycloalkyl, lower alkenyl, lower alkynyl, aryl, and heterocyclic group mentioned here, have the same meanings as the halogen (ix), lower alkyl (iv), cycloalkyl (vii), lower alkenyl (v), lower alkynyl (vi), aryl (viii), and heterocyclic group (x) defined later, respectively. Substituents of the substituted lower alkyl, substituted lower alkenyl, and substituted lower alkynyl have the same meanings as substituents of the substituted lower alkyl (xiii) defined later, respectively, and substituents of the substituted cycloalkyl, substituted aryl, and substituted heterocyclic group have the same meanings as substituents of the substituted cycloalkyl (xvi) defined later, respectively.

Specifically, examples of the substituted sterol include substituted sterol having hydroxy at 3-position, for example, substituted estrone such as 2-hydroxyestrone, 2-methoxyestrone, 4-hydroxyestrone, 6 alpha-hydroxyestrone, 1 alpha-hydroxyestrone, 15 alpha-hydroxyestrone, and 15 beta-hydroxyestrone; substituted estradiol such as 2-hydroxy-17 beta-estradiol, 2-methoxy-17 beta-estradiol, 4-hydroxy-17 beta-estradiol, 6 alpha-hydroxy-17 beta-estradiol, 7 alpha-hydroxy-17 beta-estradiol, 16 alpha-hydroxy-17 alpha-estradiol, 16 beta-hydroxy-17 alpha-estradiol, 16 beta-hydroxy-17 beta-estradiol, 17 alpha-estradiol, 17 beta-estradiol, and 17 alpha-ethynyl-17 beta-estradiol; substituted estriol such as 2-hydroxyestriol, 2-methoxyestriol, 4-hydroxyestriol, 6 alpha-hydroxyestriol, and 7 alpha-hydroxyestriol; and substituted dehydroepiandrosterone such as 6 alpha-hydroxydehydroepiandrosterone, 7 alpha -hydroxydehydroepiandrosterone, 16 alpha-hydroxydehydroepiandrosterone, and 16 beta-hydroxydehydroepiandrosterone. These substituted sterol may further have the above-mentioned substituents.

(iv) Examples of the lower alkyl include linear or branched alkyl having 1 to 20 carbon atoms, e.g. methyl, ethyl, propyl, isopropyl, butyl, isobutyl, sec-butyl, tert-butyl, pentyl, isopentyl, neopentyl, hexyl, heptyl, octyl, isooctyl, decyl, undecyl, dodecyl, tridecyl, tetradecyl, pentadecyl, and eicocyl.

(v) Examples of the lower alkenyl include linear or branched alkenyl having 2 to 8 carbon atoms, e.g. vinyl, allyl, 1-propenyl, butenyl, pentenyl, hexenyl, heptenyl, and octenyl.

(vi) Examples of the lower alkynyl include linear or branched alkynyl having 2 to 8 carbon atoms, e.g. ethynyl, propynyl, butynyl, pentynyl, hexynyl, heptynyl, and octynyl.

(vii) Examples of the cycloalkyl include cycloalkyl having 3 to 8 carbon atoms, e.g. cyclopropyl, cyclobutyl, cyclopentyl, cyclohexyl, cycloheptyl, and cyclooctyl.

(viii) Examples of the aryl include aryl having 6 to 14 carbon atoms, e.g. phenyl, naphthyl, and anthryl.

(ix) Examples of the halogen include fluorine, chlorine, bromine, and iodine atoms.

(x) Examples of the heterocyclic group include an aliphatic heterocyclic group and an aromatic heterocyclic group.

Examples of the aliphatic heterocyclic group include a 5- or 6-membered monocyclic group containing at least one atom selected from a nitrogen atom, an oxygen atom, and a sulfur atom, and a bicyclic or tricyclic fused ring which is formed by condensation 3- to 8-membered rings and which contains at least one atom selected from a nitrogen atom, an oxygen atom, and a sulfur atom. Specific examples include tetrahydropyranyl, pyranyl, tetrahydrofuranyl, pyrrolidinyl, piperidino, piperidyl, perhydroazepinyl, perhydroazocinyl, morpholino, morpholinyl, thiomorpholino, thiomorpholinyl, piperazinyl, homopiperazinyl, oxazolinyl, dioxolanyl, imidazolidinyl, imidazolinyl, pyrazolidinyl, indolinyl, 1-oxo-1,3-dihydroisoindolyl, 1,1-dioxo-2,3-dihydrobenz[d]isothiazolyl, 2-pyrrolinyl, 2-pyrrolidonyl, 3-pyrrolidonyl, 2-piperidonyl, 3-piperidonyl, 4-piperidonyl, perhydro-2-azepinonyl, perhydro-3-azepinonyl, perhydro-4-azepinonyl, 2-thiazolidonyl, 4-thiazolidonyl, 2-oxazolidonyl, 4-oxazolidonyl, succinimide, glutarimide, hydantoinyl, thiazolidinedionyl, oxazolidinedionyl, and the like.

Examples of the aromatic heterocyclic group include a 5- or 6-membered monocyclic group containing at least one atom selected from an nitrogen atom, an oxygen atom, and a sulfur atoms, and bicyclic or tricyclic fused ring which is formed by condensation of 3- to 8-membered rings and contains at least one atom selected from a nitrogen atom, an oxygen atom, and a sulfur atom. Specific examples include furyl, thienyl, pyrrolyl, imidazolyl, pyrazolyl, triazolyl, tetrazolyl, oxazolyl, isoxazolyl, thiazolyl, isothiazolyl, 1,2,3-oxadiazolyl, 1,2,4-oxadiazolyl, 1,3,4-oxadiazolyl, 1,2,3-thiadiazolyl, 1,2,4-thiadiazolyl, 1,3,4-thiadiazolyl, furazanyl, pyridyl, pyrimidinyl, pyrazinyl, pyridazinyl, quinolyl, isoquinolyl, quinazolinyl, phthalazinyl, purinyl, indolyl, isoindolyl, 2-pyridonyl, 4-pyridonyl, uracilyl, benzofuryl, benzothienyl, benzimidazolyl, benzoxazolyl, benzisoxazolyl, benzothiazolyl, benzisothiazolyl, 1,3-dioxo-1,3-dihydroisoindolyl, 1,1,3-trioxo-2,3-dihydrobenz[d]isothiazolyl, maleimido, phthalimido, and the like.

(xi) Examples of the heterocyclic group formed together with the adjacent nitrogen atom may contain an oxygen atom, a sulfur atom, or a nitrogen atom other than the adjacent nitrogen atom. Specific examples include pyrrolidinyl, thiazolidinyl, oxazolidinyl, piperidino, homopiperidino, piperazinyl, homopiperazinyl, pyrazolidinyl, morpholino, thiomorpholino, tetrahydroquinolyl, tetrahydroisoquinolyl, octahydroquinolyl, benzimidazolyl, indazolyl, indolyl, isoindolyl, purinyl, dihydroindolyl, pyrrolyl, pyrazolyl, triazolyl, tetrazolinyl, imidazolyl, and the like.

(xii) Substituents of the substituted heterocyclic group may be the same or different. The number of the substituents is 1 to 3, and examples of the substituents include halogen, nitro, cyano, azido, substituted or unsubstituted lower alkyl, substituted or unsubstituted cycloalkyl, substituted or unsubstituted lower alkenyl, substituted or unsubstituted lower alkynyl, substituted or unsubstituted aryl, a substituted or unsubstituted heterocyclic group, —C(═X¹)R⁵ (wherein X¹ and R⁵ have the same meanings as defined above, respectively), —NR⁹R¹⁰ {wherein R⁹ and R¹⁰ have the same meanings as defined above, respectively), —OR¹⁶ (wherein R¹⁶ has the same meaning as defined above), —S(O)mR¹⁸ (wherein m and R¹⁸ have the same meanings as defined above, respectively), and —SO2R¹⁹ (wherein R¹⁹ has the same meaning as defined above).

The halogen, lower alkyl, cycloalkyl, lower alkenyl, lower alkynyl, aryl, and heterocyclic group mentioned here, have the same meanings as the halogen (ix), lower alkyl (iv), cycloalkyl (vii), lower alkenyl (v), lower alkynyl (vi), aryl (viii), and heterocyclic group (x) defined above, respectively. The substituents of the substituted lower alkyl, substituted lower alkenyl, and substituted lower alkynyl have the same meanings as substituents of the substituted lower alkyl (xiii) defined later, respectively, and the substituents of the substituted cycloalkyl, substituted aryl, and substituted heterocyclic group have the same meanings as substituents of the substituted cycloalkyl (xvi) defined later, respectively.

(xiii) The substituents of the substituted lower alkyl, substituted lower alkenyl, and substituted lower alkynyl may be the same or different. The number of substituents is 1 to 3, and examples of the substituents include halogen, nitro, cyano, azido, lower alkenyl, lower alkadienyl, lower alkatrienyl, lower alkynyl, (lower alkoxy)lower alkoxy, substituted or unsubstituted cycloalkyl, substituted or unsubstituted aryl, a substituted or unsubstituted heterocyclic group, —C(═X^{1A})R^{5A} [wherein X^{1A} has the same meaning as X¹ defined above, and R^{5A} represents a hydrogen atom, lower alkyl, substituted or unsubstituted cycloalkyl, lower alkenyl, lower alkynyl, substituted or unsubstituted aryl, a substituted or unsubstituted heterocyclic group, substituted or unsubstituted aralkyl, substituted or unsubstituted heteroarylalkyl, —NR^{6A}R^{7A} (wherein R^{6A} and R^{7A} may be the same or different and each represents a hydrogen atom, lower alkyl, substituted or unsubstituted cycloalkyl, lower alkenyl, substituted or unsubstituted aryl, a substituted or unsubstituted heterocyclic group, substituted or unsubstituted aralkyl, or substituted or unsubstituted heteroarylalkyl), —OR^{8A} (wherein R^{8A} represents a hydrogen atom, lower alkyl, substituted or unsubstituted cycloalkyl, lower alkenyl, substituted or unsubstituted aryl, a substituted or unsubstituted heterocyclic group, substituted or unsubstituted aralkyl, or substituted or unsubstituted heteroarylalkyl), or —SR^{8Aa} (wherein R^{8Aa} has the same meaning as R^{8A} defined above)], —NR^{9A}R^{10A} {wherein R^{9A} and R^{1A} may be the same or different and each represents a hydrogen atom, lower alkyl, substituted or unsubstituted cycloalkyl, lower alkenyl, substituted or unsubstituted aryl, a substituted or unsubstituted heterocyclic group, substituted or unsubstituted aralkyl, substituted or unsubstituted heteroarylalkyl, —C(═X^{2A})R^{11A} (wherein X^{2A} and R^{11A} have the same meanings as X^{1A} and R^{4A} defined above, respectively), or —SO₂R^{12A} [wherein R^{12A} represents lower alkyl, substituted or unsubstituted cycloalkyl, lower alkenyl, substituted or unsubstituted aryl, a substituted or unsubstituted heterocyclic group, substituted or unsubstituted aralkyl, substituted or unsubstituted heteroarylalkyl, —NR^{13A}R^{14A} (wherein R^{13A} and R^{14A} have the same meanings as R^{6A} and R^{7A} defined above, respectively), or —OR^{15A} (wherein R^{15A} has the same meaning as R^{8A} defined above)]}, —OR^{16A} [wherein R^{16A} represents a hydrogen atom, lower alkyl, substituted or unsubstituted cycloalkyl, lower alkenyl, substituted or unsubstituted aryl, a substituted or unsubstituted heterocyclic group, substituted or unsubstituted aralkyl, substituted or unsubstituted heteroarylalkyl, or —SO₂R^{17A} (wherein R^{17A} has the same meaning as R^{12A} defined above)], —S(O)ₘₐR^{18A} (wherein ma represents 0 or 1, R^{18A} represents a hydrogen atom, lower alkyl, substituted or unsubstituted cycloalkyl, lower alkenyl, substituted or unsubstituted aryl, a substituted or unsubstituted heterocyclic group, substituted or unsubstituted aralkyl, or substituted or unsubstituted heteroarylalkyl), or —SO₂R^{19A} (wherein R^{19A} has the same meaning as R^{12A} defined above).

The halogen, lower alkyl, lower alkenyl, lower alkynyl, cycloalkyl, aryl, and heterocyclic group mentioned here, have the same meanings as the halogen (ix), lower alkyl (iv), lower alkenyl (v), lower alkynyl (vi), cycloalkyl (vii), aryl (viii), and heterocyclic group (x) described above, respectively. Examples of the lower alkadienyl (xiv) include alkadienyl having 4 to 8 carbon atoms, e.g. 1,3-butadienyl, 1,3-pentadienyl, 1,3-hexadienyl, 2,4-hexadienyl, and 1,3-octadienyl. Examples of the lower alkatrienyl (xv) include alkatrienyl having 6 to 8 carbon atoms, e.g. 1,3,5-hexatrienyl and 1,3,5-octatrienyl. A lower alkyl moiety of the (lower alkoxy)lower alkoxy has the same meaning as the lower alkyl (iv) defined above. The alkylene moieties of the (lower alkoxy)lower alkoxy, aralkyl, and heteroarylalkyl have the same meanings as the group formed by removing one hydrogen atom from lower alkyl (iv) defined above. Aryl moiety of the aralkyl group has the same meaning as the aryl (viii) defined above, and heteroaryl moiety of the heteroarylalkyl has the same meaning as the aromatic heterocyclic group in the heterocyclic group (x) defined above.

Substituents of the substituted cycloalkyl, substituted aryl, substituted heterocyclic, substituted aralkyl, and substituted heteroarylalkyl mentioned here, have the same meanings as the substituents of the substituted cycloalkyl (xvi) defined later, respectively.

(xiv) The substituents of the substituted cycloalkyl, substituted aryl, and substituted heterocyclic group formed together with the adjacent nitrogen atom may be the same or different. The number of the substituents is 1 to 3, and examples of the substituents include lower alkyl, halogen, nitro, cyano, azido, lower alkenyl, lower alkadienyl, lower alkatrienyl, lower alkynyl, (lower alkoxy)lower alkoxy, cycloalkyl, aryl, 4-sulfamoyloxybenzyl, a heterocyclic group, —C(═X^{1B})R^{5B} [wherein X^{1B} has the same meaning as X¹ defined above, and R^{5B} represents a hydrogen atom, lower alkyl, cycloalkyl, lower alkenyl, lower alkynyl, aryl, a heterocyclic group, —NR^{6B}R^{7B} (wherein R^{6B} and R^{7B} may be the same or different and each represents a hydrogen atom, lower alkyl, cycloalkyl, lower alkenyl, aryl, or a heterocyclic group), —OR^{8Ba} (wherein R^{8B} represents a hydrogen atom, lower alkyl, cycloalkyl, lower alkenyl, aryl, or a heterocyclic group), or —SR^{8Ba} (wherein R^{8Ba} has the same meaning as R^{8B} defined above)], —NR^{9B}R^{0B} {wherein R^{9B} and R^{10B} may be the same or different and each represents a hydrogen atom, lower alkyl, cycloalkyl, lower alkenyl, aryl, a heterocyclic group, —C(═X^{2B} )R^{11B} (wherein X^{2B} and R^{11B} have the same meanings as X^{1B} and R^{5B} defined above, respectively), or —SO₂R^{12B} [wherein R^{12B} represents lower alkyl, cycloalkyl, lower alkenyl, aryl, a heterocyclic group, —NR^{13B} R^{14B} (wherein R^{13B} and R^{14B} have the same meanings as R^{6B} and R^{7B} defined above, respectively), or —OR^{15B} (wherein R^{15B} has the same meaning as R^{8B} defined above)]}, —OR^{16B} [wherein R^{16B} represents a hydrogen atom, lower alkyl, cycloalkyl, lower alkenyl, aryl, a heterocyclic group, or —SO₂R^{17B} (wherein R^{17B} has the same meaning as R^{12B} defined above)], —S(O)^{mb}R^{18B} (wherein mb is 0 or 1, R^{18B} represents a hydrogen atom, lower alkyl, cycloalkyl, lower alkenyl, aryl, or a heterocyclic group), or —SO₂R^{19B} (wherein R^{19B} has the same meaning as R^{12B} defined above).

The halogen, lower alkyl, lower alkenyl, lower alkadienyl, lower alkatrienyl, lower alkynyl, cycloalkyl, aryl, and heterocyclic group mentioned here, have the same meanings as the halogen (iX), lower alkyl (iv), lower alkenyl (v), lower alkadienyl (xiv), lower alkatrienyl (xv), lower alkynyl (vi), cycloalkyl (vii), aryl (viii), and heterocyclic group (x), respectively. Lower alkyl moiety of the (lower alkoxy)lower alkoxy has the same meaning as the lower alkyl (iv) defined above, and alkylene moiety of the (lower alkoxy)lower alkoxy has the same meaning as the group formed by removing one hydrogen atom from lower alkyl (iv) defined above.

Examples of production methods for the above-mentioned effective ingredients in the steroid-sulfatase inhibitors according to the present invention will now be described.

For example, the following compounds are prepared according to the respective documents: estrone-3-methylthiophosphonate, estrone-3-methylphosphonate, estrone-3-phenylphosphonothioate, and estrone-3-phenylphosphonate [Cancer Research, vol. 53, p. 298 (1993); Bioorganic & Medicinal Chemistry Letters, vol. 3, p. 313 (1993); U.S. Pat. No. 5,604,215]; estrone-3-sulfamate derivatives [Journal of Medicinal Chemistry, vol. 37, p. 219 (1994)]; 3-desoxyestrone-3-sulfonate derivatives [Steroids, vol. 58, p. 106 (1993); The Journal of Steroid Biochemistry and Molecular Biology, vol. 50, p. 261 (1994)]; 3-desoxyestrone-3-methylsulfonate derivatives [Steroids, vol. 60, p. 299 (1995)]; estrone-3-amino derivatives [The Journal of Steroid Biochemistry and Molecular Biology, vol. 59, p. 83 (1996); U.S. Pat. Nos. 5,571,933 and 5,866,603]; vitamin D3 derivatives [The Journal of Steroid Biochemistry and Molecular Biology, vol. 48, p. 563 (1994)]; dehydroepiandrosterone derivatives [The Journal of Steroid Biochemistry and Molecular Biology, vol. 45, p. 383 (1993); Biochemistry, 36: 2586 (1997)]; estrone-3-sulfamate modifications [The Journal of Steroid Biochemistry and Molecular Biology, vol. 64, p. 269 (1998); WO98/24802; WO98/32763]; 17-alkylestradiol derivatives [Bioorganic & Medicinal Chemistry Letters, vol. 8, p. 1891 (1998); Journal of Medicinal Chemistry, vol. 42, p. 2280 (1999)]; 3-substituted-D-homo-1,3,5,(10)-estratriene derivatives (WO98/11124; WO99/27935); estrone modifications [WO98/42729; WO99/27936; Canadian Journal of Physiology and Pharmacology vol. 76, p. 99 (1998)]; 17 beta -(N-alkylcarbamoyl)estra-1,3,5(10)-triene-3-sulfamate and 17 beta -(N-alkanoylamino)estra-1,3,5(10)-triene-3-sulfamate [Steroids, vol. 63, p. 425 (1998); WO99/03876]; estrone modifications at the 17-position (WO99/33858); tetrahydronaphthol derivatives [Journal of Medicinal Chemistry, vol. 37, p. 219 (1994)]; 4-methylcoumarin-7-sulfamate [Cancer Research, vol. 56, p. 4950 (1996); WO97/30041] tyramine derivatives and phenol derivatives [Cancer Research, vol. 57, p. 702 (1997); Biochemistry, vol. 36, p. 2586 (1997); The Journal of Steroid Biochemistry and Molecular Biology, vol. 68, p. 31 (1999); U.S. Pat. No. 5,567,831]; flavonoid [The Journal of Steroid Biochemistry and Molecular Biology, vol. 63, p. 9 (1997); WO97/32872]; 4-hydroxytamoxifen derivatives [The Journal of Steroid Biochemistry and Molecular Biology, vol. 45, p. 383 (1993); Bioorganic & Medicinal Chemistry Letters, vol. 9, p. 141 (1999)]; isoflavone derivatives [The Journal of Steroid Biochemistry and Molecular Biology, vol. 69, p. 227 (1999)]; and chroman derivatives (WO99/52890).

Furthermore, WO93/05064, WO01/02349, WO97/30041 WO01/36398, and WO00/43408 disclose compounds having a steroid-sulfatase inhibiting activity that can be used in the present invention, and these compounds can be prepared according to methods disclosed therein.

For hormone therapy, agents that can (a) inhibit the production of estrogen or androgen, (b) block estrogen from binding to an estrogen receptor, (c) block androgen from binding to an androgen receptor, or (d) inhibit the secretion of estrogen or luteinizing hormone may be used. Examples of these agents are antiestrogen agents, aromatase inhibitors, antiandrogen agents, LH-RH agonists, and progesterone products, and they may be used alone or in combination.

Examples of the antiestrogen agents include compositions comprising tamoxifen, ICI-182780 (trade name; Faslodex, generic name; fulvestrant), toremifene, or pharmaceutically acceptable salts thereof as active ingredients.

Examples of the aromatase inhibitors include compositions comprising amino-glutathione, anastrozole, letrozole, exemestane, vorozole, fadrozole, or pharmaceutically acceptable salts thereof as active ingredients.

Examples of the antiandrogen agents include compositions comprising flutamide, bicalutamide, nilutamide, cyproterone, or pharmaceutically acceptable salts thereof as active ingredients.

Examples of the LH-RH agonists include compositions comprising luprolide, goserelin, or pharmaceutically acceptable salts thereof as active ingredients.

Examples of the progesterone products include compositions comprising megestrol acetate, medroxyprogesterone acetate, or pharmaceutically acceptable salts thereof as active ingredients.

Examples of the chemotherapy agents include compositions comprising adriamycin, cyclophosphamide, paclitaxel, docetaxel, vinorelbine, fluorouracil, irinotecan, methotrexate, or pharmaceutically acceptable salts thereof as active ingredients.

The pharmaceutically acceptable salts of the effective ingredients that constitute the steroid-sulfatase inhibitors, agents for hormone therapy, and agents for chemotherapy are, for example, pharmaceutically acceptable acid addition salts, metal salts, ammonium salts, organic amine addition salts, and amino acid addition salts. Examples of the acid addition salts include inorganic acid salts, e.g. hydrochloride, sulfate, and phosphate; and organic acid salts, e.g. acetate, maleate, fumarate, tartrate, citrate, lactate, and succinate. Examples of the metal salts include alkali-metal salts, e.g. sodium salt and potassium salt; alkaline-earth metal salts, e.g. magnesium salts and calcium salts; aluminium salts, and zinc salts. Examples of ammonium salts include ammonium salts and tetramethylammonium salts. Examples of organic amine addition salts include addition salts of morpholine and piperidine. Examples of amino acid addition salts include addition salts of lysine, glycine, phenylalanine, aspartic acid, and glutamic acid.

Steroid-sulfatase inhibitors and agents for hormone therapy and/or agents for chemotherapy agents used in therapeutic agents and pharmaceutical compositions for hormone-dependent cancers according to the present invention may be administered alone or in combination as preparations containing their active ingredients. Particularly, a combination of two to four preparations is preferable. When the preparations are used or administered in combination, they may be used or administered together or separately at an interval.

These preparations can be manufactured by a conventional process using a pharmaceutically acceptable diluent, excipient, disintegrant, lubricant; binder, surfactant, water, saline, vegetable-oil solubilizer, isotonic agent, preservative, or antioxidant in addition to each active ingredient.

When the preparations are administered in combination, for example, a first component comprising (a) the steroid-sulfatase inhibitor and a second component comprising (b) the agent for hormone therapy and/or agent for chemotherapy are separately prepared as described above and made into a kit. By utilizing such a kit, different preparations can be administered together or separately at an interval to one subject by the same route or different routes. The second component may be further separated into several components, preferably, two or three components.

The kit is composed of at least two containers (e.g. vials, bags) and contents (i.e. the first and second components). The material and the shape of the containers are not limited, but the containers must prevent the contents, i.e. the components, from degrading due to external temperature or light during the storage, and should be made from a material that does not elute its chemical constituents. The first component and the second component are administerable dosage forms so as to be administered through different routes (e.g. tubes) or the same route. A preferable example is a kit for injection. For example, the containers of the first and second components are formed to connect to a bag containing an infusion solution so that each of the components is mixed with the infusion solution.

A method for treating hormone-dependent cancers according to the present invention can be performed similarly to the above-mentioned utilization or administration of the steroid-sulfatase inhibitor and the agent for hormone therapy and/or agent for chemotherapy used as the therapeutic agent for hormone-dependent cancers. Namely, the method can be performed by preparing the steroid-sulfatase inhibitor and the agent for hormone therapy and/or agent for chemotherapy so as to contain their active ingredients and by administering alone or in combination, preferably, in a combination of two to four preparations. When the preparations are administered in combination, they may be administered together or separately at an interval and may also be administered in the form of a kit as described above.

The efficiency of hormone-dependent cancer treatment by the combined administration of a steroid-sulfatase inhibitor and an agent for hormone therapy will be explained in detail by referring to Experimental Examples.

### FIELD OF INVENTION

This invention relates to novel compounds for use as steroid sulphatase inhibitors, and pharmaceutical compositions containing them.

### BACKGROUND AND PRIOR ART

Steroid precursors, or pro-hormones, having a sulphate group in the 3-position of the steroid nucleus, referred to hereinafter simply as steroid sulphates, are known to play an important part as intermediates in steroid metabolism in the human body. Oestrone sulphate and dehydroepiandrosterone (DHA) sulphate, for example, are known to play an important role as intermediates in the production, in the body, of oestrogens such as oestrone and oestradiol. Oestrone sulphate, in particular, is known, for example, to represent one of the major circulating oestrogen precursors particularly in post-menopausal women and oestrone sulphatase activity in breast tumours is 100-1000 fold greater than that of other enzymes involved in oestrogen formation (James et al., Steroids, 50, 269-279 (1987)).

Not only that, but oestrogens such as oestrone and oestradiol, particularly the over-production thereof, are strongly implicated in malignant conditions, such as breast cancer, see Breast Cancer, Treatment and Prognosis: Ed. R. A. Stoll, pp. 156-172, Blackwell Scientific Publications (1986), and the control of oestrogen production is the specific target of many anti cancer therapies, both chemotherapy and surgical, e.g. oöphorectomy and adrenalectomy. So far as endocrine therapy is concerned, efforts have so far tended to concentrate on aromatase inhibitors, i.e. compounds which inhibit aromatase activity, which activity is involved, as the accompanying oestrogen metabolic flow diagram (FIG. 1) shows, in the conversion of androgens such as androstenedione and testosterone to oestrone and oestradiol respectively.

In recently published International Application WO91/13083 a proposal has been made to target a different point in the oestrogen metabolic pathway, or rather two different points, that is to say the conversion of DHA sulphate and oestrone sulphate to DHA and oestrone, respectively, by steroid sulphatase activity, and using 3-monoalkylthiophosphonate steroid esters as a steroid sulphatase inhibitor, more especially oestrone-3-monomethyl-thiophosphonate.

### OBJECTS OF THE INVENTION

A first object of the present invention is to provide new compounds capable of inhibiting steroid sulphatase activity in vitro and in vivo.

A second object of the present invention is to provide new compounds having improved activity as steroid sulphatase inhibitors both in vitro and in vivo.

A third object of the invention is to provide pharmaceutical compositions effective in the treatment of oestrogen dependent tumours.

A fourth object of the invention is to provide pharmaceutical compositions effective in the treatment of breast cancer.

A fifth object of the invention is to provide a method for the treatment of oestrogen dependent tumours in mammals, especially humans.

A sixth object of the invention is to provide a method for the treatment of breast cancer in mammals and especially in women.

### SUMMARY OF INVENTION

The invention is based on the discovery of novel compounds having steroid sulphatase inhibitory activity, in some cases, with extremely high activity levels.

In one aspect, the present invention provides a method of inhibiting steroid sulphatase activity in a subject in need of same.

In another aspect, the present invention provides compounds and compositions useful in that method of inhibiting steroid sulphatase activity.

The method of the present invention comprises administering to said subject a steroid sulphatase inhibiting amount of a ring system compound; which ring system compound comprises a ring to which is attached a sulphamate group of the formula 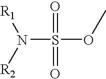
wherein each of R₁ and R₂ is independently selected from H, alkyl, alkenyl, cycloalkyl and aryl, or together represent alkylene optionally containing one or more hetero atoms or groups in the alkylene chain, and wherein said compound is an inhibitor of an enzyme having steroid sulphatase activity (E.C.3.1.6.2); and if the sulphamate group of said compound is replaced with a sulphate group to form a sulphate compound and incubated with a steroid sulphatase enzyme (E.C.3.1.6.2) at a pH 7.4 and 37° C. it would provide a Kₘ value of less than 50 μM.

The compounds that are useful in the method of the present invention are sulphamic acid ester ring system compounds, the sulphate of which is a substrate for enzymes having steroid sulphatase (EC 3.1.6.2) activity, the N-alkyl and N-aryl derivatives of those sulphamic acid esters, and their pharmaceutically acceptable salts.

In one aspect of the present invention, compounds for use in the method of the present invention are the sulphamic acid esters of polycyclic alcohols, being polycyclic alcohols the sulphate of which is a substrate for enzymes having steroid sulphatase (EC 3.1.6.2) activity, the N-alkyl and N-aryl derivatives of those sulphamic acid esters, and their pharmaceutically acceptable salts.

For one aspect of the present invention, broadly speaking, the novel compounds of this invention are compounds of the Formula (I) 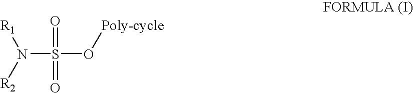
where:

R₁ and R₂ are each independently selected from H, alkyl, cycloalkyl, alkenyl and aryl, or together represent alkylene optionally containing one or more hetero atoms or groups in the alkylene chain; and the group —O-Polycycle represents the residue of a polycyclic alcohol, the sulphate of which is a substrate for enzymes having steroid sulphatase activity (EC 3.1.6.2).

As used herein the reference to polycyclic alcohols, the sulphate of which is a substrate for enzymes having steroid sulphatase activity refers to polycyclic alcohols, the sulphate of which, viz: the derivatives of the Formula: 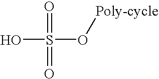
when incubated with steroid sulphatase EC 3.1.6.2 at pH 7.4 and 37° C. provides a Kₘ value of less than 50 moles.

### BRIEF DESCRIPTION OF DRAWINGS

The activity of the present compounds as steroid sulphatase inhibitors is illustrated in the accompanying drawings, in which:

FIG. 1 is a schematic chart showing the metabolic pathways, enzymes and steroid intermediates associated with the production of oestradiol in vivo.

FIG. 2 is a histogram showing the dose-dependent inhibitory effect of oestrone-3-sulphamate on steroid sulphatase activity in human MCF-7 cells in vitro.

FIG. 3 is a histogram showing the dose-dependent inhibitory effect of oestrone-3-N,N-dimethylsulphamate on steroid sulphatase activity in human MCF-7 cells in vitro.

FIG. 4 is a graph comparing the log dose-response curves for oestrone-3-sulphamate and oestrone-3-N,N-dimethylsulphamate on steroid sulphatase activity in human MCF-7 cells in vitro.

FIG. 5 is a graph showing the dose-dependent inhibitory effect of oestrone-3-sulphamate, together with its IC₅₀ value (concentration required to produce 50% inhibition), on steroid sulphatase activity in human placental microsomes in vitro.

FIG. 6 shows the structures of oestrone (1), oestrone sulphate (2), oestrone-3-sulphamate (otherwise known as “EMATE”) (3) and steroid sulphamates (4-5) (See Example 13 and WO 97/30041).

FIG. 7 shows the structures of 7-hydroxycoumarin (11), 7-(sulphoxy)-4-methylcoumarin (12) and coumarin sulphamates (13-16) (See Example 13 and WO 97/30041).

FIG. 8 shows the sulphation of 7-hydroxy-4-methylcoumarin; pyridine/SO3-pyridine complex, NaOH in MeOH (Route a) (See Example 13 and WO 97/30041).

FIG. 9 shows the sulphamoylation of 7-hydroxy-4-methylcoumarin; NaH/DMF, H2NSO2Cl in toluene (Route b) (See Example 13 and WO 97/30041).

FIG. 10 shows the dose-dependent inhibition of oestrone sulphatase in intact MCF-7 breast cancer cells by coumarin-7-O-sulphamate (13), 4-methylcoumarin-7-O-sulphamate (14), 3,4,8-trimethyl-coumarin-7-O-sulphamate (15) and 4-(trifluoromethyl)coumarin-7-O-sulphamate (16) (See Example 13 and WO 97/30041).

FIG. 11 shows the time-dependent and the concentration-dependent inactivation of oestrone sulphatase by 4-methyl-coumarin-7-O-sulphamate (14) (See Example 13 and WO 97/30041).

FIG. 12 is a graph (% inhibition vs. coumate) (See Example 13 and WO 97/30041).

FIGS. 13 and 14 present Formulae (A) to (H) with Formulae (A), (B) and (C) presented in FIG. 13 and Formulae (D), (E), (F), (G) and presented in FIG. 14 (See Example 13. Preferably, in Formula (A), R₁13 R₆ are independently selected from H, halo, hydroxy, sulphamate, alkyl and substituted variants or salts thereof, but wherein at least one of R₁—R⁶ is a sulphamate group; and wherein X is any one of O, S, NH, a substituted N, CH₂, or a substituted C. Preferably X is O. Preferably, if the sulphamate group of the compound were to be replaced with a sulphate group to form a sulphate compound then that sulphate compound would be hydrolysable by an enzyme having steroid sulphatase (E.C.3.1.6.2) activity. In a highly preferred embodiment, the compound is not hydrolysable by an enzyme having steroid sulphatase (E.C.3.1.6.2) activity. In Formula (B) R₁—R₆ are independently selected from H, halo, hydroxy, sulphamate, alkyl and substituted variants or salts thereof; but wherein at least one of R₁—R₆ is a sulphamate group. The alkyl group(s) in Formula (A) or Formula (B) can be any suitable linear or branched alkyl group which may be saturated or unsaturated and/or substituted or non-substituted. The alkyl group may even be a cyclic alkyl group. For example, at least two of R₁—R₆ are linked to form a further cyclic component. Preferably R₁—R₅ are independently selected from H, alkyl and haloalkyl; preferably wherein R₁—R₅ are independently selected from H, C₁₋₆ alkyl and C₁₋₆haloalkyl. Preferably R₁—R₅ are independently selected from H, C₁₋₃ alkyl and C1-3 haloalkyl. Preferably R₁—R₅ are independently selected from H, methyl and halomethyl. Preferably R₆ is OSO₂NH₂. In Formula (A) or Formula (B), two or more of R₁—R₆ may be linked together to form an additional cyclic structure. A typical example of such a compound has the general Formula (C), wherein any one of R₃—R₆ is a sulphamate group, and wherein n is an integer. Typically, R6 is a sulphamate group. A typical sulphamate group is —OS(O)(O)—NH₂. Preferably n is an integer of from 3 to 10, preferably from 3 to 7. Optionally, the group (CH₂)n of Formula (C) can be a substituted alkyl chain. Typical compounds falling within the general Formula (C) are shown in FIG. 14 as compound (D) (where n=3), compound (E) (where n=4), compound (F) (where n=5), compound (G) (where n=6), compound (H) (where n=7). For these compounds, R₆ is a sulphamate group of the formula —OS(O)(O)—NH₂ and each of R₃—R₅ is H. The term “sulphamate” includes an ester of sulphamic acid, or an ester of an N-substituted derivative of sulphamic acid, or a salt thereof. Thus, the term includes functional groups of the formula: —O—S(O)(O)—N(R₇)(R₈) where R₇ and R₈ are independently selected from H, halo, linear or branched alkyl which may be saturated or unsaturated and/or substituted or non-substituted, aryl, or any other suitable group. Preferably, at least one or R₇ and R₈ is H. In a preferred embodiment, each of R₇ and R₈ is H. See also WO 97130041).

FIG. 15 shows a schematic diagram of some enzymes involved in the in situ synthesis of oestrone from oestrone sulfate, oestradiol and androstenedione (See Example 14 and WO 97/32872, see also FIG. 1. FIG. 15 shows the origin of oestrogenic steroids in postmenopausal women; “ER” denotes Oestrogen Receptor, “DHA/-S” denotes Dehydroepiandrosterone/-Sulfate; “DHA-STS” denotes DHA-sulphatase; “Adiol-STS” denotes Adiol Sulphatase; and “17B-HSD” denotes Oestradiol 17B-hydroxysteroid dehydrogenases).

FIGS. 16a, 16b, 16c, and FIGS. 17 to 23 depict chemical formulae (See Example 14 and WO 97/32872. In a preferred embodiment, the compounds comprise a first ring structure and a sulphamoyl group, which first ring structure may be substituted and/or unsaturated. The first ring structure is preferably a phenolic ring structure, which may be substituted. The compounds may further comprise a second ring structure, which may be substituted and/or unsaturated. The compounds may preferably be a sulphamate of a flavone, an isoflavone or a flavanone, or a sulphamate of a benzoflavone, e.g., FIG. 23 wherein R is H or OH; and, the invention also encompasses substituted variants of the sulphamate of the benzoflavone of FIG. 23. With regard to FIGS. 16a, 16b, 16c and 17 to 22, it is generally preferred that in formula I, A represents the first ring structure, B represents the third ring structure, D represents the second ring structure, C is an optional double bond, E is a link joining the second ring structure to the third ring structure, X represents a suitable first group, and Y represents a suitable second group, wherein any one of ring structures A, B, and D is a phenolic ring, and any one of ring structures A, B and D has bound thereto a sulphamate group. Each of the ring structures can independently comprise from 3 to 20 atoms in the ring, preferably 4 to 8 atoms in the ring; and, preferably ring A and ring D comprise 6 atoms in the ring. A further cyclic group may be linked to ring A or D. This cyclic group may be linked to two spaced-apart atoms in ring A or ring D, such as the structure shown in FIG. 23. Preferably the first ring structure and the second ring structure are substituted. Preferably any one of ring structures A and D has bound thereto a sulphamate group. Preferably, each of the first ring and the second ring is a homogeneous ring structure, i.e., the ring is made up of the same atoms. Preferably, each of the first ring and the second ring comprises only carbon atoms in the ring. Preferably X is C═O. Preferably the compound has the general formula II wherein F represents a phenolic ring structure (the first ring structure), J represents the third ring structure, I represents a phenolic ring structure (the second ring structure), G is an optional double bond, H is a link joining the second ring structure to the third ring structure, and Y represents a suitable second group, and any one of ring structures F, J and I has bound thereto a sulphamate group. Preferably the third ring structure is a heterogeneous ring structure, i.e., different atoms are in the ring. Preferably, Y is O. Preferably, any one of the ring structures F and I has bound thereto a sulphamate group. Preferably link E or link H is a bond. Preferably, the compound is a sulphamate of any one of a flavone, an isoflavone or a flavanone. Preferably, the compound is a compound of formula IV, V or VI, wherein R₁—R₁₂ are independently selected from H, OH, a halogen, an amine, an amide, a sulphonamine, a sulphonamide, any other sulphur containing group, a saturated or unsaturated C₁₋₁₀ ally, an aryl group, a saturated or unsaturated C₁₋₁₀ ether, a saturated or unsaturated C₁₋₁₀ ester, a phosphorus containing group, and wherein at least one of R₁—R₁₂ is a sulphamate group. Preferably the sulphamate group has the general formula OSO₂NR₁₃R₁₄ wherein R₁₃ and R₁₄ are independently selected from H, OH, a halogen, a saturated or unsaturated C₁₋₁₀ alkyl, an aryl group, a saturated or unsaturated C₁₋₁₀ ether, a saturated or unsaturated C₁₋₁₀ ester, and, each of R₁₃ and R₁₄ may be other suitable groups. Preferably the compound is a compound of formula IV, V or VI, wherein R₁—R₁₂ are independently selected from H, OH, OSO₂NR₁₃R₁₄, O—CH₃; wherein at least one of R₁—R₁₂ is OSO₂NR₁₃R₁₄ and R₁₃ and R₁₄ are as defined above. Preferably, at least one of R₁₃ and R₁₄ is H; and preferably each is H. Preferably, the compound is a sulphamate of any one of the flavone of formula VII, the isoflavone of formula VIII, or the flavanone of formula IX Preferably the compound is a sulphamate of any of formulae VII, VIII or IX. Preferably the compound is a sulphamate of a flavone, isoflavone or flavanone wherein the suphamoyl group is on the C4′ atom of the flavone, isoflavone or flavanone. The C4′ position is shown in formula III. Preferably, the compound is a flavonoid or flavanoid sulphamate. Preferably, if the sulphamate group of the compound were to be replaced with a sulphate group so as to form a sulphate compound then that sulphate compound would be hydrolysable by an enzyme having steroid sulphatase (E.C.3.1.6.2) activity. The compound may have one or more sulphamate groups. For example, the compound may be mono-sulphamate or a bis-sulphamate. For instance, in FIGS. 20-22, R₃ and R₄ may be each a sulphamate).

FIG. 24 presents a bar graph of inhibition of oestrone sulphatase (See Example 14 and WO 97/32872).

FIGS. 25 to 34 show compounds of the Formulae I to X, respectively (See Example 15 and WO 98/24802. With reference to FIGS. 25 to 34: In Formula I; A is a first group; B is an aryl ring structure having at least 4 carbon atoms in the ring and wherein the ring B is substituted in at least the 2 position and/or the 4 position with an atom or group other than H; X is a sulphamate group; wherein group A and ring B together are capable of mimicking the A and B rings of oestrone; and wherein group A is attached to at least one carbon atom in ring B. The term “mimic” as used herein means having a similar or different structure but having a similar functional effect. In other words, group A and ring B together of the compounds of the present invention are bio-isosteres of the A and B rings of oestrone. Preferably, the sulphamate group is at position 3 of the ring B. Preferably, the ring B has six carbon atoms in the ring. Preferably, the compound has the Formula II; wherein X is the sulphamate group; A is the first group; R₁ and/or R₂ is a substituent other than H; wherein R₁ and R₂ may be the same or different but not both being H; and wherein optionally group A is attached to at least one other carbon atom in ring B. Preferably, group A is additionally attached to the carbon atom at position 1 of the ring B. Preferably, group A and ring B are a steroid ring structure or a substituted derivative thereof. Preferably, the compound has the Formula IV; wherein X is the sulphamate group; R₁ and/or R₂ is a substituent other than H; wherein R₁ and R₂ may be the same or different but not both being H; and wherein Y is a suitable linking group. Suitable linking groups for Y include groups made up of at least any one or more of C, O, N, and S. The linking groups can also comprise H. The linking group may also increase the size of the ring (i.e. the D ring). Preferably, however, the D ring comprising Y is a five-membered ring. Preferably, Y is —CH₂— or —C(O)—. Preferably, Y is —C(O)—. Preferably, the compound has the Formula V; wherein X is the sulphamate group; R₁ and/or R₂ is a substituent other than H; and wherein R₁ and R₂ may be the same or different but not both being H. The term “sulphamate” as used herein includes an ester of sulphamic acid, or an ester of an N-substituted derivative of sulphamic acid, or a salt thereof. Preferably, the sulphamate group has the Formula III. In Formula III, each of R₃ and R₄ is independently selected from H or a hydrocarbyl group. The term “hydrocarbyl group” as used herein means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. A non-limiting example of a hydrocarbyl group is an acyl group. In one preferred embodiment of the present invention, the hydrocarbyl group for the sulphamate group is a hydrocarbon group. Here the term “hydrocarbon” means any one of an alkyl group, an alkenyl group, an alkynyl group, which groups may be linear, branched or cyclic, or an aryl group. The term hydrocarbon also includes those groups but wherein they have been optionally substituted. If the hydrocarbon is a branched structure having substituent(s) thereon, then the substitution may be on either the hydrocarbon backbone or on the branch; alternatively the substitutions may be on the hydrocarbon backbone and on the branch Preferably, R₃ and R₄ are independently selected from H or alkyl, cycloalkyl, alkenyl and aryl, or together represent alkylene, wherein the or each alkyl or cycloalkyl or alkenyl or optionally contain one or more hetero atoms or groups. When substituted, the N-substituted compounds of this invention may contain one or two N-alkyl, N-alkenyl, N-cycloalkyl or N-aryl substituents, preferably containing or each containing a maximum of 10 carbon atoms. When R₃ and/or R₄ is alkyl, the preferred values are those where R₃ and R₄ are each independently selected from lower alkyl groups containing from 1 to 5 carbon atoms, that is to say methyl, ethyl, propyl etc. Preferably R₃ and R₄ are both methyl. When R₃ and/or R₄ is aryl, typical values are phenyl and tolyl (-PhCH₃; o-, m- or p-). Where R₃ and R₄ represent cycloalkyl, typical values are cyclopropyl, cyclopentyl, cyclohexyl etc. When joined together R₃ and R₄ typically represent an alkylene group providing a chain of 4 to 6 carbon atoms, optionally interrupted by one or more hetero atoms or groups, e.g. —O— or —NH— to provide a 5-, 6- or 7-membered heterocycle, e.g. morpholino, pyrrolidino or piperidino. Within the values alkyl, cycloalkyl, alkenyl and aryl Applicants include substituted groups containing as substituents therein one or more groups which do not interfere with the sulphatase inhibitory activity of the compound in question. Exemplary non-interfering substituents include hydroxy, amino, halo, alkoxy, alkyl and aryl. In some preferred embodiments, at least one of R₃ and R₄ is H. In some further preferred embodiments, each of R₃ and R₄ is H. Preferably, each of R₁ and R₂ is independently selected from H, alkyl, cycloalkyl, alkenyl, aryl, substituted alkyl, substituted cycloalkyl, substituted alkenyl; substituted aryl, any other suitable hydrocarbyl group, a nitrogen containing group, a S containing group, a carboxy containing group. Likewise, here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. A non-limiting example of a hydrocarbyl group is an acyl group. Preferably, each of R₁ and R₂ is independently selected from H, C₁₋₆ alkyl, C₁₋₆ cycloalkyl, C₁₋₆ alkenyl, substituted C₁₋₆ alkyl, substituted C₁₋₆ cycloalkyl, substituted-C₁₋₆ alkenyl, substituted aryl, a nitrogen containing group, a S containing group, or a carboxy group having from 1-6 carbon atoms. Likewise, here within the values alkyl, cycloalkyl, alkenyl and aryl Applicants include substituted groups containing as substituents therein one or more groups which do not interfere with the sulphatase inhibitory activity of the compound in question. Exemplary non-interfering substituents include hydroxy, amino, halo, alkoxy, alkyl and aryl. Preferably, each of R₁ and R₂ is independently selected from H, C₁₋₆ alkyl, C₁₋₆ alkenyl, a nitrogen containing group, or a carboxy group having from 1-6 carbon atoms. Preferably, each of R₁ and R₂ is independently selected from H, C₁₋₆ alkyl, C₁₋₆ alkenyl, NO₂, or a carboxy group having from 1-6 carbon atoms. Preferably, each of R₁ and R₂ is independently selected from H, C₃ alkyl, C₃ alkenyl, NO₂, or H₃CHO. Preferably, the compound is any one of the Formulae V-DC. Preferably, for some applications, the compound is further characterised by the feature that if the sulphamate group were to be substituted by a sulphate group to form a sulphate derivative, then the sulphate derivative would be hydrolysable by an enzyme having steroid sulphatase (E.C.3.1.6.2) activity—i.e. when incubated with steroid sulphatase EC 3.1.6.2 at pH 7.4 and 37 C. In one preferred embodiment, if the sulphamate group of the compound were to be replaced with a sulphate group to form a sulphate compound then that sulphate compound would be hydrolysable by an enzyme having steroid sulphatase (E.C. 3.1.6.2) activity and would yield a Kₘ value of less than 50 mmoles when incubated with steroid sulphatase EC 3.1.6.2 at pH 7.4 and 37 C. In another preferred embodiment, if the sulphamate group of the compound were to be replaced with a sulphate group to form a sulphate compound then that sulphate compound would be hydrolysable by an enzyme having steroid sulphatase (E.C. 3.1.6.2) activity and would yield a Kₘ value of less than 50 mmoles when incubated with steroid sulphatase EC 3.1.6.2 at pH 7.4 and 37 C. In a highly preferred embodiment, the compound of the present invention is not hydrolysable by an enzyme having steroid sulphatase (E.C. 3.1.6.2) activity. Preferably the group A and the ring B together—hereinafter referred to as “group A/ring B combination”—will contain, inclusive of all substituents, a maximum of about 50 carbon atoms, more usually no more than about 30 to 40 carbon atoms. A preferred group A/ring B combination has a steroidal ring structure, that is to say a cyclopentanophenanthrene skeleton. Preferably, the sulphamyl or substituted sulphamyl group is attached to that skeleton in the 3-position. Thus, according to a preferred embodiment, the group A/ring B combination is a substituted or unsubstituted, saturated or unsaturated steroid nucleus. A suitable steroid nucleus is a substituted (i.e. substituted in at least the 2 and/or 4 position and optionally elsewhere in the steroid nucleus) derivative of any one of: oestrone, 2-OH-oestrone, 2-methoxy-oestrone, 4-OH oestrone, 6a-OH-oestrone, 7a-OH-oestrone, 16a-OH-oestrone, 16b-OH-oestrone, oestradiol, 2-OH-17b-oestradiol, 2-methoxy-17b-oestradiol, 4-OH-17b-oestradiol, 6a-OH-17b-oestradiol, 7a-OH-17b-oestradiol, 16a-OH-17a-oestradiol, 16b-OH-17a-oestradiol, 16b-H-17b-oestradiol, 17a-oestradiol, 17b-oestradiol, 17a-ethinyl-17b-oestradiol, oestriol, 2-OH-oestriol, 2-methoxy-oestriol, 4-OH-oestriol, 6a-OH-oestriol, 7a-OH-oestriol, dehydroepiandrosterone, 6a-OH-dehydroepiandrosterone, 7a-OH-dehydroepiandrosterone, 16a-OH-dehydroepiandrosterone, 16b-OH-dehydroepiandrosterone. In general terms the group A/ring B combination may contain a variety of non-interfering substituents. In particular, the group A/ring B combination may contain one or more hydroxy, alkyl especially lower (C₁-C₆) alkyl, e.g. methyl, ethyl, n-propyl, isopropyl, n-butyl, sec-butyl, tert-butyl, n-pentyl and other pentyl isomers, and n-hexyl and other hexyl isomers, alkoxy especially lower (C₁-C₆) alkoxy, e.g. methoxy, ethoxy, propoxy etc., alkenyl, e.g. ethenyl, or halogen, e.g. fluoro substituents. The group A/ring B combination may even be a non-steroidal ring system. A suitable non-steroidal ring system is a substituted (i.e. substituted in at least the 2 and/or 4 position and optionally elsewhere in the ring system) derivative of any one of: diethylstilboestrol, stilboestrol. When substituted, the N-substituted compounds of this invention may contain one or two N-alkyl, N-alkenyl, N-cycloalkyl or N-aryl substituents, preferably containing or each containing a maximum of 10 carbon atoms. When R₁ and/or R₂ and/or R₃ and/or R₄ is alkyl, the preferred values are those where each of R₁ and R₂ and R₃ and R₄ is independently selected from lower alkyl groups containing from 1 to 6 carbon atoms, that is to say methyl, ethyl, propyl etc. When R₁ and/or R₂ and/or R₃ and/or R₄ is aryl, typical groups are phenyl and tolyl (-PhCH₃; o-, m- or p-). Where R₁ and/or R₂ and/or R₃ and/or R₄ represent cycloalkyl, typical values are cyclopropyl, cyclopentyl, cyclohexyl etc. When joined together R₃ and R₄ typically represent an alkylene group providing a chain of 4 to 6 carbon atoms, optionally interrupted by one or more hetero atoms or groups, e.g. —O— or —NH— to provide a 5-, 6- or 7-membered heterocycle, e.g. morpholino, pyrrolidino or piperidino. Within the values alkyl, cycloalkyl, alkenyl and aryl we include substituted groups containing as substituents therein one or more groups which do not interfere with the sulphatase inhibitory activity of the compound in question. Examples of non-interfering substituents include hydroxy, amino, halo, alkoxy, allyl and aryl. Applicants have also surprisingly found that when the compound has the Formula IV where Y═—CH₂— it is not necessary for the compound to be substituted in the 2 and 4 ring positions, i.e., R₁ and R₂ may both be hydrogen. In one embodiment of this aspect, any of the ring positions (including R₁ and R₂, but excluding Y) may be substituted. Thus, according to another aspect of the present invention there is provided a sulphamate compound of the Formula X and wherein X is a sulphamate group, and Y is CH₂ and optionally any other H attached directly to the ring system is substituted by another group. X may be as described above. Any replacement for H on the ring system may be any one of the substituents described above in relation to R₁ and R₂. In an especially preferred embodiment there is no substitution on the ring system, i.e., a compound of Formula IV where Y is —CH₂— and R₁ and R₂ are both H).

FIGS. 35 to 38 show methods of preparing compounds of the present invention (See Example 15 and WO 98/24802. The sulphamate compounds of the present invention may be prepared by reacting an appropriate alcohol with a sulfamoyl chloride, R₃R₄NSO₂Cl. Preferred conditions for carrying out the reaction are as follows: Sodium hydride and a sulfamoyl chloride are added to a stirred solution of the alcohol in anhydrous dimethyl formamide at 0 C. Subsequently, the reaction is allowed to warm to room temperature whereupon stirring is continued for a further 24 hours. The reaction mixture is poured onto a cold saturated solution of sodium bicarbonate and the resulting aqueous phase is extracted with dichloromethane. The combined organic extracts are dried over anhydrous MgSO₄. Filtration followed by solvent evaporation in vacuo and co-evaporated with toluene affords a crude residue which is further purified by flash chromatography. Preferably, the alcohol is derivatised, as appropriate, prior to reaction with the sulfamoyl chloride. Where necessary, functional groups in the alcohol may be protected in known manner and the protecting group or groups removed at the end of the reaction).

FIG. 39 shows a graph illustrating the in vivo inhibition of oestrone sulphatase by NOMATE (0.1 mg/Kg/day for five days) (See Example 15 and WO 98/24802).

FIG. 40 shows a graph illustrating the lack of effect of NOMATE (0.1 mg/Kg/day for five days) on uterine weights in ovariectomised rats (See Example 15 and WO 98/24802).

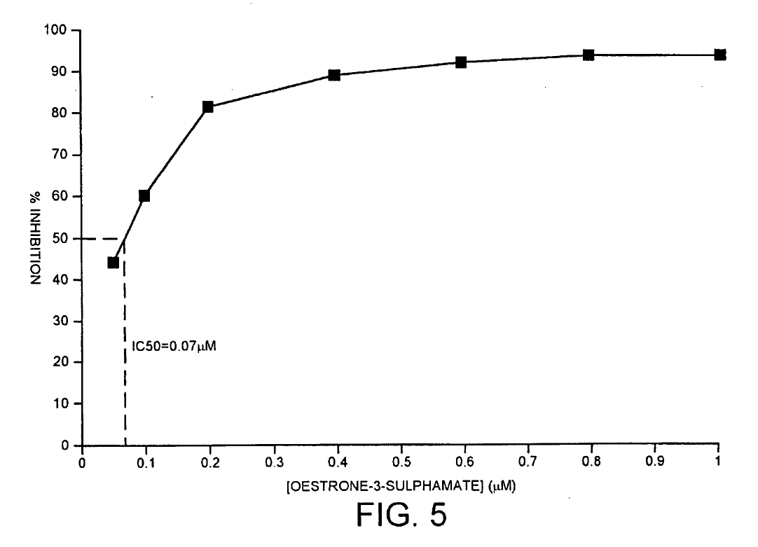

FIG. 5 is a graph showing the dose-dependent inhibitory effect of oestrone-3-sulphamate, together with its IC₅₀ value (concentration required to produce 50% inhibition), on steroid sulphatase activity in human placental microsomes in vitro.

### DETAILED DESCRIPTION

Thus, in one aspect, the present invention provides a method of inhibiting steroid sulphatase activity in a subject in need of same, the method comprising administering to said subject a steroid sulphatase inhibiting amount of a ring system compound; which ring system compound comprises a ring to which is attached a sulphamate group of the formula 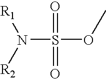
wherein each of R₁ and R₂ is independently selected from H, alkyl, alkenyl, cycloalkyl and aryl, or together represent alkylene optionally containing one or more hetero atoms or groups in the alkylene chain; and wherein said compound is an inhibitor of an enzyme having steroid sulphatase activity (E.C.3.1.6.2); and if the sulphamate group of said compound is replaced with a sulphate group to form a sulphate compound and incubated with a steroid sulphatase enzyme (E.C.3.1.6.2) at a pH 7.4 and 37° C. it would provide a Kₘ value of less than 50 μM.

In one aspect the present invention provides, as novel compounds, the sulphamic acid esters of polycyclic alcohols, being polycyclic alcohols the sulphate of which is a substrate for enzymes having steroid sulphatase activity in accordance with the definition already provided, and their N-alkyl, N-cycloalkyl, N-alkenyl and N-aryl derivatives. These compounds are of Formula I hereinbefore given.

Preferably the polycyclic group will contain, inclusive of all substituents, a maximum of about 40 carbon atoms, more usually no more than about 30.

Preferred polycycles are those containing a steroidal ring structure, that is to say a cyclopentanophenanthrene skeleton. Preferably, the sulphamyl or substituted sulphamyl group is attached to that skeleton in the 3-position, that is to say are compounds of the Formula II: 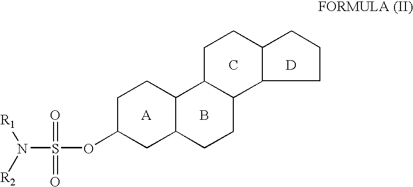
where R₁ and R₂ are as above defined and the ring system ABCD represents a substituted or unsubstituted, saturated or unsaturated steroid nucleus, preferably oestrone or dehydroepiandrosterone.

Other suitable steroid ring systems are:

substituted oestrones, viz:

| | | | |
|---|---|---|---|
| 2-OH-oestrone | 2-methoxy-oestrone | 4-OH-oestrone | 6α-OH- |
| | | | oestrone |
| 7α-OH-oestrone | 16α-OH-oestrone | 16β-OH-oestrone | |

oestradiols and substituted oestradiols, viz:

| | | |
|---|---|---|
| 2-OH-17β- | 2-methoxy-17β-oestradiol | 4-OH-17β-oestradiol |
| oestradiol | | |
| 6α-OH-17β- | 7α-OH-17β-oestradiol | 16α-OH-17α- |
| oestradiol | | oestradiol |
| 16β-OH-17α- | 16β-OH-17β-oestradiol | 17α-oestradiol |
| oestradiol | | |
| 17β-oestradiol | 17α-ethinyl-17β- | |
| | oestradiol | |

oestriols and substituted oestriols, viz:

| | | |
|---|---|---|
| oestriol | 2-OH-oestriol | 2-methoxy-oestriol |
| 4-OH-oestriol | 6α-OH-oestriol | 7α-OH-oestriol |

substituted dehydroepiandrosterones, viz:

| | |
|---|---|
| 6α-OH-dehydroepiandrosterone | 7α-OH-dehydroepiandrosterone |
| 16α-OH-dehydroepiandrosterone | 16β-OH-dehydroepiandrosterone |

In general terms the steroid ring system ABCD may contain a variety of non-interfering substituents. In particular, the ring system ABCD may contain one or more hydroxy, alkyl especially lower (C₁-C₆) alkyl, e.g. methyl, ethyl, n-propyl, isopropyl, n-butyl, sec-butyl, tert-butyl, n-pentyl and other pentyl isomers, and n-hexyl and other hexyl isomers, alkoxy especially lower (C₁-C₆) alkoxy, e.g. methoxy, ethoxy, propoxy etc., alkinyl, e.g. ethinyl, or halogen, e.g. fluoro substituents.

Suitable non-steroidal ring systems (i.e. ring system compounds) include:

diethylstilboestrol, stilboestrol and other ring systems providing sulfates having Kₘ values of less than 50 μmoles with steroid sulphatase EC3.1.6.2.

Examples of some non-steroidal ring systems are presented in the Examples section.

When substituted, the N-substituted compounds of this invention may contain one or two N-allyl, N-alkenyl, N-cycloalkyl or N-aryl substituents, preferably containing or each containing a maximum of 10 carbon atoms. When R₁ and/or R₂ is alkyl, the preferred values are those where R₁ and R₂ are each independently selected from lower alkyl groups containing from 1 to 5 carbon atoms, that is to say methyl, ethyl, propyl etc. Preferably R₁ and R₂ are both methyl. When R₁ and/or R₂ is aryl, typical values are phenyl and tolyl (-PhCH₃; o-, m- or p-). Where R₁ and R₂ represent cycloalkyl, typical values are cyclopropyl, cyclopentyl, cyclohexyl etc. When joined together R₁ and R₂ typically represent an alkylene group providing a chain of 4 to 6 carbon atoms, optionally interrupted by one or more hetero atoms or groups, e.g. -0- or —NH— to provide a 5-, 6- or 7)-membered heterocycle, e.g. morpholino pyrrolidino or piperidino.

Within the values alkyl, cycloalkyl, alkenyl and aryl we include substituted groups is containing as substituents therein one or more groups which do not interfere with the sulphatase inhibitory activity of the compound in question. Exemplary non-interfering substituents include hydroxy, amino, halo, alkoxy, allyl and aryl.

Most preferred are compounds of the Formula III and IV: 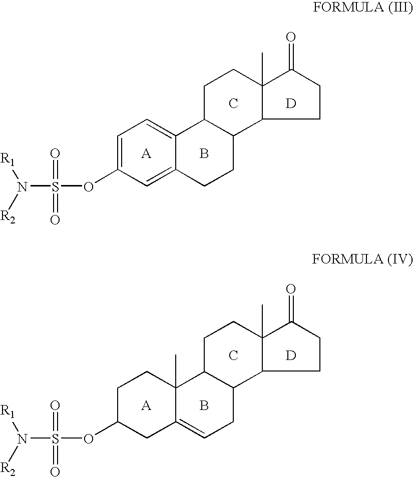
where R₁ and R₂ are H or C₁-C₅ alkyl, i.e. oestrone-3-sulphamate and dehydroepiandrosterone-3-sulphamate and their N-C₁-C₅) alkyl derivatives, especially the dimethyl derivatives, R₁═R₂═CH₃.

The sulphamic acid esters of this invention are prepared by reacting the polycyclic alcohol, e.g. oestrone or dehydroepiandrosterone, with a sulfamoyl chloride R₁R₂NSO₂Cl, i.e. the Reaction Scheme I

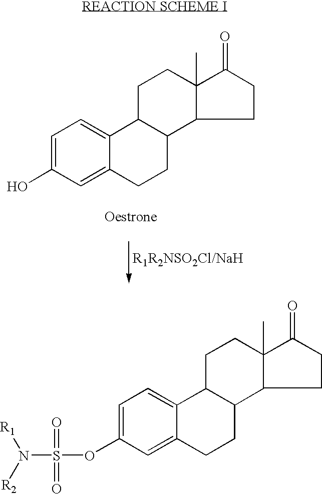

Conditions for carrying out Reaction Scheme I are as follows:

Sodium hydride and a sulphamoyl chloride are added to a stirred solution of oestrone in anhydrous dimethyl formamide at 0° C. Subsequently, the reaction is allowed to warm to room temperature whereupon stirring is continued for a further 24 hours. The reaction mixture is poured onto a cold saturated solution of sodium bicarbonate and the resulting aqueous phase is extracted with dichloromethane. The combined organic extracts are dried over anhydrous MgSO₄. Filtration followed by solvent evaporation in vacuo and co-evaporation with toluene affords a crude residue which is further purified by flash chromatography.

Where necessary, functional groups in the polycyclic alcohol (sterol) may be protected in known manner and the protecting group or groups removed at the end of the reaction.

For pharmaceutical administration, the steroid sulphatase inhibitors of this invention can be formulated in any suitable manner utilising conventional pharmaceutical formulating techniques and pharmaceutical carriers, exipients, diluents etc. and usually for parenteral administration. Approximate effective dose rates are in the range 100 to 800 mg/day depending on the individual activities of the compounds in question and for a patient of average (70 kg) bodyweight. More usual dosage rates for the preferred and more active compounds will be in the range 200 to 800 mg/day, more preferably, 200 to 500 mg/day, most preferably from 200 to 250 mg/day. They may be given in single dose regimes, split dose regimes and/or in multiple dose regimes lasting over several days. For oral administration they may be formulated in tablets, capsules, solution or suspension containing from 100 to 500 mg of compound per unit dose. Alternatively and preferably the compounds will be formulated for parenteral administration in a suitable parenterally administrable carrier and providing single daily dosage rates in the range 200 to 800 mg, preferably 200 to 500, more preferably 200 to 250 mg. Such effective daily doses will, however, vary depending on inherent activity of the active ingredient and on the bodyweight of the patient, such variations being within the skill and judgement of the physician.

For particular applications, it is envisaged that the steroid sulphatase inhibitors of this invention may be used in combination therapies, either with another sulphatase inhibitor, or, for example, in combination with an aromatase inhibitor, such as for example, 4-hydroxyandrostenedione (4-OHA).

The invention is illustrated by the following preparative Examples and test data:

### EXAMPLE 1

### Preparation of oestrone-3-sulphamate

Sodium hydride (60% dispersion; 2 eq) and suiphamoyl chloride (2 eq) were added to a stirred solution of oestrone (1 eq) in anhydrous dimethyl formamide at 0° C. Subsequently, the reaction was allowed to warm to room temperature whereupon stirring was continued for a further 24 hours.

The reaction mixture was poured onto a cold saturated solution of sodium bicarbonate and the resulting aqueous phase was extracted with dichloromethane. The combined organic extracts were dried over anhydrous MgSO₄. Filtration followed solvent evaporation in vacuo and co-evaporation with toluene afforded a crude residue which is further purified by flash chromatography.

Analysis showed the following data:

δ¹H (270 MHz; CD₃OD): 0.91 (s, 3H, C₁₈-Me), 1.40-2.55 (series of m, 13H), 2.90-2.92 (m, 2H), 7.04 (br d, 2H, J=10.44 Hz), 7.33 (br d, 1H, J=8.42 Hz).

δ¹³C (67.8 MHz; CD₃OD): 14.53 (q, C₁₈-Me), 22.80 (t), 27.24 (t), 27.73 (t), 30.68 (t), 33.05 (t), 37.01 (t), 39.76 (d), 45.73 (s, C₁₈), 51.86 (d), 120.76 (d), 123.54 (d), 127.89 (d), 139.83 (s), 150.27 (s), 223.87 (s, C═O).

m/z (%): 349 (9) (m⁺), 270 (100), 213 (26), 185 (43), 172 (31), 159 (21), 146 (36), 91 (33), 69 (37), 57 (73), 43 (56), 29 (24).

Microanalysis:

| | C | H | N |
|---|---|---|---|
| Expected: | 61.87% | 6.63% | 4.01% |
| Found: | 61.90% | 6.58% | 3.95% |

### EXAMPLE 2

### Preparation of oestrone-3-N-methylsulphamate

The procedure of Example 1 was repeated save that sulphamoyl chloride was replaced by the same quantity of N-methylsulphamoyl chloride.

Analysis showed the following data:

δ¹H (270 MHz; CDCl₃): 0.91 (s, 3H, C₁₈-Me), 1.28-1.68 (m, 6H), 1.93-2.60 (series of m, 7H), 2.90-2.95 (m, 2H), 2.94 (d, 3H, J=5.13 Hz, MeN—), 4.68-4.71 (br m, exchangeable, 1H, —NH), 7.02-7.07 (m, 2H), 7.26-7.32 (m, 1H).

m/z (%): 364 [M+H]⁺

### EXAMPLE 3

### Preparation of oestrone-3-N,N-dimethylsulphamate

The procedure of Example 1 was repeated save that sulphamoyl chloride was replaced by the same quantity of N,N-dimethylsulphamoyl chloride.

Analysis showed the following data:

δ¹H (270 MHz; CDCl₃): 0.92 (s, 3H, C₁₈-Me), 1.39-1.75 (m, 5H), 1.95-2.60 (series of m, 6H), 2.82 (s, 3H, MeN—), 2.96-3.00 (m, 4H), 2.98 (s, 3H, MeN—), 7.04 (br d, 2H, J=7.69 Hz), 7.29 (br d, 1H, J=7.88 Hz).

m/z (%): 377 [M]⁺

Microanalysis:

| | C | H | N |
|---|---|---|---|
| Expected: | 63.63% | 7.21% | 3.71% |
| Found: | 63.50% | 7.23% | 3.60% |

### EXAMPLE 4

### Inhibition of Steroid Sulphatase Activity in MCF-7 cells by oestrone-3-sulphamate

Steroid sulphatase is defined as: Steryl Sulphatase EC 3.1.6.2.

Steroid sulphatase activity was measured in vitro using intact MCF-7 human breast cancer cells. This hormone dependent cell line is widely used to study the control of human breast cancer cell growth. It possesses significant steroid sulphatase activity (MacIndoe et al. Endocrinology, 123, 1281-1287 (1988); Purohit & Reed, Int. J. Cancer, 50, 901-905 (1992)) and is available in the U.S.A. from the American Type Culture Collection (ATCC) and in the U.K. (e.g. from The Imperial Cancer Research Fund). Cells were maintained in Minimal Essential Medium (MEM) (Flow Laboratories, Irvine, Scotland) containing 20 mM HEPES, 5% foetal bovine serum, 2 mM glutanile, non-essential amino acids and 0.075% sodium bicarbonate. Up to 30 replicate 25 cm² tissue culture flasks were seeded with approximately 1×10⁵ cells/flask using the above medium. Cells were grown to 80% confluency and medium was changed every third day.

Intact monolayers of MCF-7 cells in triplicate 25 cm² tissue culture flasks were washed with Earle's Balanced Salt Solution (EBSS from ICN Flow, High Wycombe, U.K.) and incubated for 3-4 hours at 37° C. with 5 pmol (7×10⁵ dpm) [6,7-³H]oestrone-3-sulphate (specific activity 60 Ci/mmol from New England Nuclear, Boston, Mass., U.S.A.) in serum-free MEM (2.5 ml) together with oestrone-3-sulphamate (11 concentrations: 0; 1 fM; 0.01 pM; 0.1 pM; 1 pM; 0.01 nM; 0.1 nM; 1 nM; 0.01 μM; 0.1 μM; 1 μM). After incubation each flask was cooled and the medium (1 ml) was pipetted into separate tubes containing [¹⁴C]oestrone (7×10³ dpm) (specific activity 97 Ci/mmol from Amersham International Radiochemical Centre, Amersham, U.K.). The mixture was shaken thoroughly for 30 seconds with toluene (5 ml). Experiments showed that >90% [¹⁴C]oestrone and <0.1% [³H]oestrone-3-sulphate was removed from the aqueous phase by this treatment. A portion (2 ml) of the organic phase was removed, evaporated and the ³H and ¹⁴C content of the residue determined by scintillation spectrometry. The mass of oestrone-3-sulphate hydrolysed was calculated from the ³H counts obtained (corrected for the volumes of the medium and organic phase used, and for recovery of [¹⁴C]oestrone added) and the specific activity of the substrate. Each batch of experiments included incubations of microsomes prepared from a sulphatase-positive human placenta (positive control) and flasks without cells (to assess apparent non-enzymatic hydrolysis of the substrate). The number of cell nuclei per flask was determined using a Coulter Counter after treating the cell monolayers with Zaponin. One flask in each batch was used to assess cell membrane status and viability using the Trypan Blue exclusion method (Phillips, H. J. (1973) In: Tissue culture and applications, [eds: Kruse, D. F. & Patterson, M. K.]; pp. 406-408; Academic Press, New York).

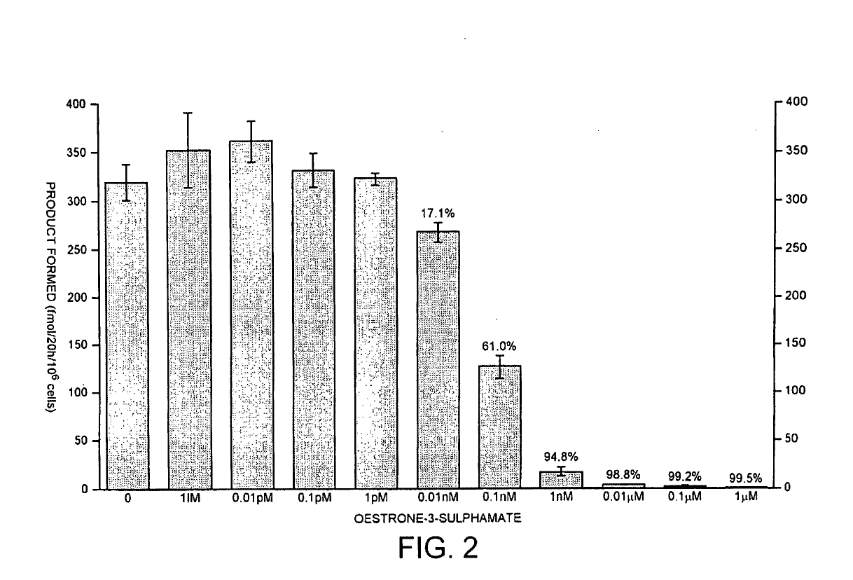

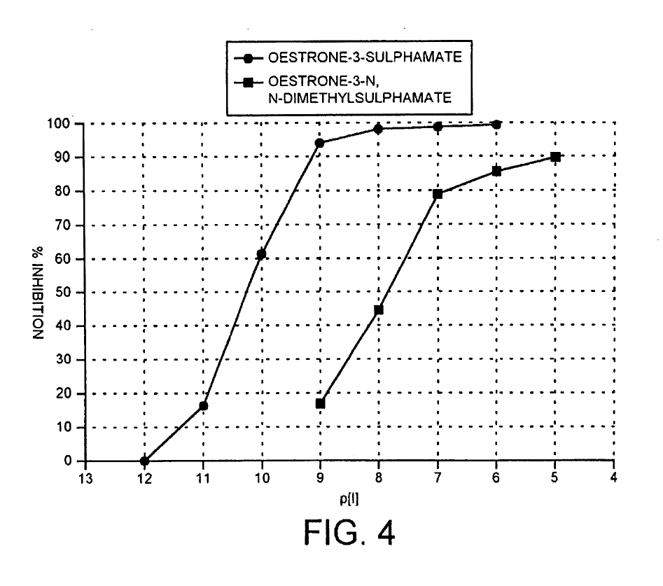

Data for oestrone-3-sulphamate are shown in Table I and FIGS. 2 and 4. Results for steroid sulphatase activity are expressed as the mean ±1 S.D. of the total product (oestrone+oestradiol) formed during the incubation period (20 hours) calculated for 10⁶ cells and, for values showing statistical significance, as a percentage reduction (inhibition) over incubations containing no oestrone-3-sulphamate. Unpaired Student's t-test was used to test the statistical significance of results.

TABLE I
Steroid Sulphatase Activity in MCF-7 cells
in the presence of Oestrone-3-sulphamate

| Oestrone-3- | | Steroid Sulphatase | % reduction over |
|---|---|---|---|
| sulphamate | | Activity ¶ (fmol/20 | control (% |
| concentration | | hr/10⁶ cells) | inhibition) |
| 0 | (control) | 319.7 ± 18.5 | — |
| 1 | fM | 353.3 ± 39.0 | — |
| 0.01 | pM | 362.3 ± 21.2 | — |
| 0.1 | pM | 330.7 ± 17.8 | — |
| 1 | pM | 321.8 ± 6.2 | — |
| 0.01 | nM | 265.1 ± 11.0* | 17.2% |
| 0.1 | nM | 124.8 ± 12.4*** | 60.9% |
| 1 | nM | 16.49 ± 4.7*** | 95.0% |
| 0.01 | μM | 3.92 ± 0.4*** | 98.8% |
| 0.1 | μM | 2.53 ± 1.1*** | 99.2% |
| 1 | μM | 1.68 ± 0.7*** | 99.5% |

¶ mean ± 1 S.D. n = 3
*p ≦ 0.05
***p ≦ 0.001

### EXAMPLE 5

### Inhibition of Steroid Sulphatase Activity in MCF-7 cells by oestrone-3-N,N-dimethylsulphamate

An identical experimental protocol to that described in Example 4 was used to generate results for oestrone-3-N,N-dimethylsulphamate except that incubations contained oestrone-3-N,N-dimethylsulphamate (5 concentrations: 0; 0.001 μM; 0.01 μM; 0.1 μM; 1 μM) in place of oestrone-3-sulphamate.

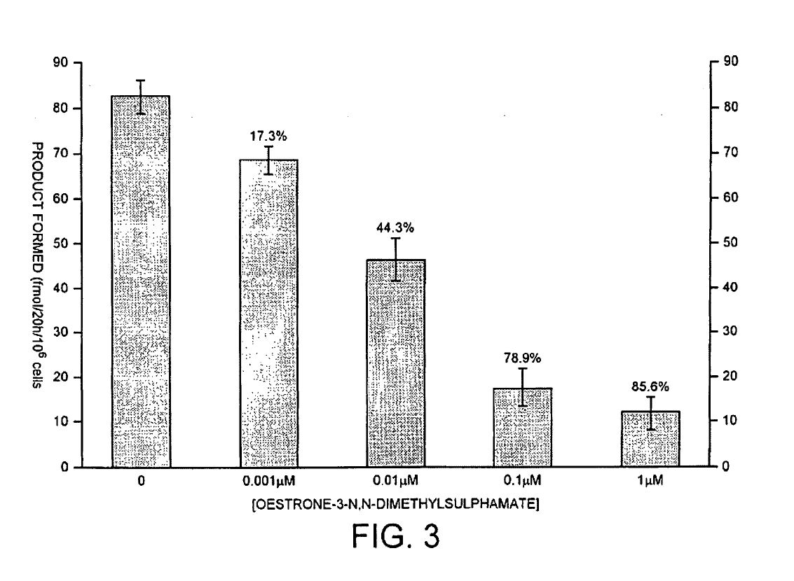

Results for oestrone-3-N,N-dimethylsulphamate are shown in Table II and FIG. 3 and are expressed in an identical manner to Table I and FIG. 2 respectively. Additionally the log dose-response curve is compared with oestrone-3-sulphamate in FIG. 4.

TABLE II
Steroid Sulphatase Activity in MCF-7 cells in the
presence of Oestrone-3-N,N-dimethylsulphamate

| Oestrone-3-N,N- | | Steroid Sulphatase | % reduction over |
|---|---|---|---|
| dimethylsulphamate | | Activity ¶ (fmol/20 | control (% |
| concentration | | hr/10⁶ cells) | inhibition) |
| 0 | (control) | 82.63 ± 3.6 | — |
| 0.001 | μM | 68.33 ± 3.2** | 17.3% |
| 0.01 | μM | 46.0 ± 4.9*** | 44.3% |
| 0.1 | μM | 17.43 ± 4.3*** | 78.9% |
| 1 | μM | 11.89 ± 3.7*** | 85.6% |

¶ mean ± 1 S.D. n = 3
**p ≦ 0.01
***p ≦ 0.001

### EXAMPLE 6

### Inhibition of Steroid Sulphatase Activity in MCF-7 cells by pre-treatment with oestrone-3-N,N-dimethylsulphamate and oestrone-3-N,N-dimethylsulphamate

A similar experimental protocol to that described in Example 4 was used to determine the effect of pre-treating MCF-7 cells with oestrone-3-sulphamate and oestrone-3-N,N-dimethylsulphamate respectively.

Intact monolayers were initially incubated for 2 hours at 37° C. with 0.1 μM oestrone-3-sulphamate, oestrone-3-N,N-dimethylsulphamate or medium alone (control). The medium bathing the cells was then removed by aspiration and cells were washed 3 times successively with 5 ml of medium on each occasion. The resultant ‘washed’ cells were then re-suspended and incubated for 3-4 hours at 37° C. in medium containing 5 pmol (7×10⁵ dpm) [6,7-³H]oestrone-3-sulphate. All other aspects were identical to those described Examples 3 and4.

Results for oestrone-3-sulphamate and oestrone-3-N,N-dimethylsulphamate are shown in Table III and are expressed in a similar manner to Table I.

TABLE III
Steroid Sulphatase Activity in MCF-7 cells
pre-incubated with Oestrone-3-sulphamates

| | Steroid Sulphatase | % reduction over |
|---|---|---|
| | Activity ¶ (fmol/20 | control (% |
| Pre-treatment | hr/10⁶ cells) | inhibition) |
| Control | 65.4 ± 6.4 | — |
| Oestrone-3-sulphamate | 1.7 ± 0.2*** | 97.4% |
| Oestrone-3-N,N- | 53.1 ± 3.4* | 18.8% |
| dimethylsulphamate | | |

¶ mean ± 1 S.D. n = 3
*p ≦ 0.05
***p ≦ 0.001

### EXAMPLE 7

### Inhibition of Steroid Sulphatase Activity in Placental Microsomes by Oestrone-3-sulphamate

Sulphatase-positive human placenta from normal term pregnancies (Obstetric Ward, St. Mary's Hospital, London) were thoroughly minced with scissors and washed once with cold phosphate buffer (pH 7.4, 50 mM) then re-suspended in cold phosphate buffer (5 ml/g tissue). Homogenisation was accomplished with an Ultra-Turrax homogeniser, using three 10 second bursts separated by 2 minute cooling periods in ice. Nuclei and cell debris were removed by centrifuging (4° C.) at 2000 g for 30 minutes and portions (2 ml) of the supernatant were stored at −20° C. The protein concentration of the supernatants was determined by the method of Bradford (Anal. Biochem., 72, 248-254 (1976)).

Incubations (1 ml) were carried out using a protein concentration of 100 μg/ml, substrate concentration of 20 μM [6,7-³H]oestrone-3-sulphate (specific activity 60 Ci/mmol from New England Nuclear, Boston, Mass., U.S.A.) and an incubation time of 20 minutes at 37° C. Eight concentrations of oestrone-3-sulphamate were employed: 0 (i.e. control); 0.05 μM; 0.1 μM; 0.2 μM; 0.4 μM; 0.6 μM; 0.8 μM; 1.0 μM. After incubation each sample was cooled and the medium (1 ml) was pipetted into separate tubes containing [¹⁴C]oestrone (7×10³ dpm) (specific activity 97 Ci/mmol from Amersham International Radiochemical Centre, Amersham, U.K.). The mixture was shaken thoroughly for 30 seconds with toluene (5 ml). Experiments showed that >90% (¹⁴C]oestrone and <0.1% [³H]oestrone-3-sulphate was removed from the aqueous phase by this treatment. A portion (2 ml) of the organic phase was removed, evaporated and the ³H and ¹⁴C content of the residue determined by scintillation spectrometry. The mass of oestrone-3-sulphate hydrolysed was calculated from the ³H counts obtained (corrected for the volumes of the medium and organic-phase used, and for recovery of [¹⁴C]oestrone added) and the specific activity of the substrate.

Results for oestrone-3-sulphamate are shown in Table IV and FIG. 5. Results for steroid sulphatase activity are expressed in Table IV as total product (oestrone+oestradiol) formed during the incubation period (time) and as a percentage reduction (inhibition) over incubations containing no oestrone-3-sulphamate which acted as control. Results for steroid sulphatase activity are expressed in FIG. 4 as percentage reduction (inhibition) over control against concentration of oestrone-3-sulphamate and include the calculated IC₅₀ value (i.e. the concentration of oestrone-3-sulphamate which produces 50% inhibition in relation to control) of 0.07 μM.

TABLE IV
Steroid Sulphatase Activity in placental microsomes
in the presence of Oestrone-3-sulphamate

| Oestrone-3- | | Steroid Sulphatase | % reduction over |
|---|---|---|---|
| sulphamate | | Activity ¶ (pmol/hr/ | control (% |
| concentration | | 0.1 mg protein) | inhibition) |
| 0 | (control) | 768.6 | — |
| 0.05 | μM | 430.4 | 44.0% |
| 0.1 | μM | 305.9 | 60.2% |
| 0.2 | μM | 140.0 | 81.8% |
| 0.4 | μM | 83.3 | 89.2% |
| 0.6 | μM | 61.8 | 92.0% |
| 0.8 | μM | 49.2 | 93.6% |
| 1.0 | μM | 51.6 | 93.3% |

¶ mean of 2 estimates

### EXAMPLE 8

### Inhibition of Steroid Sulphatase Activity in Liver Microsome Preparations from Rats Treated with Subcutaneous Oestrone-3-sulphamate

Four groups of 3 female Wistar rats (weight range 80-110 g) were given 100 ml subcutaneous injections (once daily for 7 days, vehicle: propylene glycol) of either:

Propylene glycol (vehicle control)

Oestrone-3-sulphamate (10 mg/kg/day)

Oestrone-3-sulphate (10 mg/kg/day) (substrate control)

Oestrone-3-sulphate (10 mg/kg/day)+Oestrone-3-sulphamate (10 mg/kg/day)

On the eighth day all rats were sacrificed and livers were removed by dissection. Liver microsomal preparations were prepared by an identical protocol to that described in Example 6 except that the tissue source was rat liver and that duplicate experiments to determine steroid sulphatase activity were performed using [6,7-³H]oestrone-3-sulphate and [7-³]dehydroepiandrosterone-3-sulphate as separate substrates.

Results for steroid sulphatase activity are shown in Table V and are expressed as total product formed during the incubation period in the form of mean ±1 S.D. Results for incubations of tissue obtained from groups of rats treated with oestrone-3-sulphamate are also expressed as a percentage reduction (inhibition) in steroid sulphatase activity compared to their respective controls.

TABLE V
Steroid Sulphatase Activity in Liver Microsome Preparations
from Rats treated with subcutaneous Oestrone-3-sulphamate

| | | Steroid Sulphatase | |
|---|---|---|---|
| | Assay | Activity ¶ (nmol/30 | % reduction |
| Treatment Group | Substrate | min/200 mg protein) | over control |
| control (vehicle) | E₁-S | 20.95 ± 0.2 | — |
| E₁-SO₃NH₂ | E₁-S | 0.34 ± 0.1*** | 98.4% |
| control (E₁-S) | E₁-S | 20.6 ± 0.4 | — |
| E₁-S + E₁SO₃NH₂ | E₁-S | 0.21 ± 0.03*** | 99.0% |
| control (vehicle) | DHA-S | 1.73 ± 0.4 | — |
| E₁-SO₃NH₂ | DHA-S | 0.1 ± 0.01*** | 94.2% |
| control (E₁-S) | DHA-S | 1.71 ± 0.1 | — |
| E₁-S + E₁-SO₃NH₂ | DHA-S | 0.09 ± 0.01*** | 94.7% |

¶ mean ± 1 S.D. n = 3
***≦0.001
E₁-S = oestrone-3-sulphamate
DHA-S = dehydroepiandrosterone-3-sulphate
E₁-SO₃NH₂ = oestrone-3-N,N-dimethylsulphamate

### EXAMPLE 9

Starting with the appropriate parent compound, the ring system sulphamates according to the present invention were prepared essentially as follows. In this regard, a solution of the appropriate parent compound in anhydrous DMF was treated with sodium hydride [60% dispersion; 1.2 equiv.] at 0° C. under an atmosphere of N₂. After evolution of hydrogen had ceased, sulfamoyl chloride in toluene [excess, ca. 5 equiv.] was added and the reaction mixture was poured into brine after warming to room temperature overnight and diluting with ethyl acetate. The organic fraction was washed exhaustively with brine, dried (MgSO₄), filtered and evaporated. The crude product obtained was purified by flash chromatography and recrystallisation to give the corresponding sulfamate. All the compounds were fully characterized by spectroscopic and combustion analysis.

Example compounds are as follows:

### EXAMPLE 9a

### 4-n-Heptyl phenyl-O-sulphamate (9a)

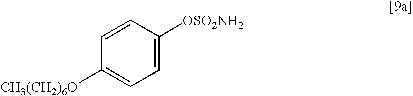

4-n-Heptyloxyphenol (1.0 g, 4.80 mmol) gave a crude product (1.41 g) which was fractionated on silica (200 g) with chloroform/acetone (8:1), and upon evaporation the second fraction gave a pale white residue (757 mg) which was recrystallized from acetone/hexane (1:5) to give 9a as white crystals (0.557 g).

Analytical results were as follows:

m.p.>42° C. (dec.)

R_{f}s=0.56, 0.69 and 0.8 for chloroform/acetone 8:1, 4:1 and 2:1 respectively

vmax (KBr) 3440, 3320 (—NH₂), 1370 (—SO₂N—) cm⁻¹

δ_{H} (acetone-d₆) (270 MHz) 0.89 (3H, t, C-4-CH₃), 1.34 (8H, m, —(CH₂)₄CH₃), 1.79 (2H, pentet, —CH₂(CH₂)₄CH₃), 4.0 (2H, t, J=6.4 Hz, —OCH₂—), 6.95 (2H, dd, J_{C-3-H and C-5-H}=2.2 Hz and J_{C-3-H} and _{C-2-H}=6.97 Hz, C-3-H and C-5-H), 7.01 (2H, br s, exchanged with D₂O, —OSO₂NH₂), 7.23 (2H, dd, J_{C-2-H and C-6-H}=2.4 Hz and J_{C-2-H and C-6-H}=6.97 Hz, C-2-H and C-6-H)

MS: m/z (+ve ion FAB in m-NBA, rel. intensity) 287.1 [100, (M)⁺], 208.2 [30, (M-SO₂NH₂)⁺] MS: m/z (−ve ion FAB in m-NBA, rel. intensity) 286.0 [100, (M−H)⁻], 96.0 (10) and 77.9 (20). Acc. MS: m/z (FAB)⁺ 288.1246 C₁₃H₂₂NO₄S requires 288.1269.

Found C, 54.2; H, 7.35; N, 4.7; C₁₃H₂₁NO₄S requires C, 54.33; H, 7.37; N, 4.87%.

Biological Data:

% Inhibition in MCF-7 Cells at 10 μM=99±2

% Inhibition in Placental Microsomes at 10 μM=40±2

% Inhibition in vivo from a single dose of 10 mg/kg=22±3

### EXAMPLE 9(b)

### (E) Methyl-γ-methyl-6-nonenamide-N-(3-methoxyphenyl-4-O-sulphamate) (9b)

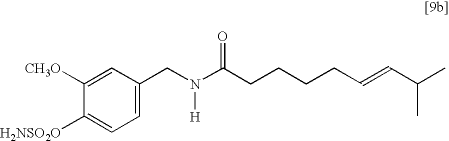

E-Capsaicin ((E)-N-(4-Hydroxy-3-methoxyphenyl)-methyl-γ-methyl-6-nonenamide) (100 mg, 0.3274 mmol) gave a beige crude product (130 mg) which was fractionated on silica (100 g) with chloroform/acetone (2:1), and upon evaporation the second fraction gave a pale white residue (85 mg) which was recrystallized from acetone/hexane (1:2) to give 9b as pale white crystals (63 mg, 50%).

Analytical results were as follows:

m.p=114-116° C.

R_{f}s=0.4 and 0.15 for chloroform/acetone 2:1 and 4:1 respectively

vmax (KBr) 3490,3300 (—NH₂), 1650 (CO), 1380 (—SO₂N—) cm⁻¹

δ_{H} (CDCl₃) (270 MHz) 0.94 (6H, d, J=6.6 Hz, 2x-CH₃), 1.4 (2H, pentet, —COCH₂CH₂— or —CH₂CH₂CH═CH—), 1.62 (2H, pentet, —CH₂CH₂CH═CH— or —COCH₂CH₂—), 2.0 (2H, q, —CH₂CH═CH—), 2.2 (3H, t, —CH₂CONH— and —CH(CH₃)₂), 3.87(3H, s, C-3-OCH₃), 4.39 (2H, d, J=5.86 Hz, ArCH₂NHCO), 5.14 (2H, br s, exchanged with D₂O, —OSO₂NH₂), 5.34 (2H, m, —CH═CH—), 5.87 (1H, t, —NHCO—), 6.84 (1H, dd, J=_{C-6-H and C-2-H} 1.92 Hz and J=_{C-6-H and C-5-H} 8.15 Hz C-6-H), 6.86 (1H, d, J=_{C-1-H and C-6-H} 1.83 Hz, C-1-H) and 7.26 (1H, d, J=_{C-5-H and C-6-H} 8.08 Hz, C-5-H).

MS: m/z (+ve ion FAB in m-NBA, rel. intensity) 385.2 [100, (M+H)⁺], 304.2 (20), 287.1 (10) MS: m/z (−ve ion FAB in m-NBA, rel. intensity) 383.1 (100, (M−H)], 302.2 (10), 95.9 (10) and 77.9 (35)

Found C, 56.2; H, 7.38; N, 7.29; C₁₈H₂₈N₂O₅S requires C, 56.23; H, 7.34; N, 7.29%.

Biological Data:

% Inhibition in MCF-7 Cells at 10 μM=99±2

% Inhibition in Placental Microsomes at 10 μM=40±2

% Inhibition in vivo from a single dose of 10 mg/kg=22±3

9(c) 2-Nitrophenol-O-sulfamate (9c)

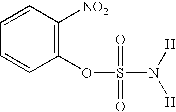

A stirred solution of 2-nitrophenol (1.391 g, 10.0 mmol) in anhydrous DMF (20 mL) was treated with sodium hydride (60% dispersion, 400 mg, 10.0 mmol) at 0° C. under an atmosphere of N₂. After evolution of hydrogen had ceased, sulfamoyl chloride (2 eq.) was added. The reaction mixture was stirred at room temperature overnight and then poured into water (150 mL). The resulting mixture was extracted with ethyl acetate (150 mL) and the organic portion separated was washed with brine (5×100 mL), dried (MgSO₄), filtered and evaporated in vacuo at 40° C. Purification by flash chromatography (ethyl acetate/hexane, 1:1) gave the crude 2-nitrophenol-O-sulfamate which was further purified by recrystallization from hot chloroform to afford the title compound as white crystals (333 mg, 745.8 μmol). The residue recovered, from the evaporation of the mother liquor, was recrystallized from chloroform/hexane to give further crops of the title compound (a total of 252 mg, 564.4 μmol, 26% overall).

Analytical results were as follows:

mp 102-103° C.

¹H NMR (270 MHz, CDCl₃) δ 5.29 (2H, br s, exchanged with D₂O, OSO₂NH₂), 7.48 (1H, m, C4-H or C5-H), 7.66 (1H, dd, J=1.5 and 8.3 Hz, C3-H or C6-H), 7.71 (1H, m, C4-H or C5-H) and 8.05 (1H, dd, J=1.5 and 8.3 Hz, C3-H or C6-H)

MS (EI, 70 eV) m/z (rel. intensity) 218(2, M⁺), 139[100, (M-SO₂NH)⁺]; MS (CI, isobutane) m/z (rel. intensity) 219[27, (M+H)⁺], 202[10, (M+H—NH₃)⁺], 140[100, (M+H—SO₂NH)⁺], 122[15, (M−OSO₂NH₂)⁺]. Anal. (C₆H₆N₂O₅S) C, H, N.

Biological Data:

% Inhibition in MCF-7 Cells at 10 μM=99±2

% Inhibition in Placental Microsomes at 10 μM=40±2

% Inhibition in vivo from a single dose of 10 mg/kg=22±3

### EXAMPLE 10

Starting with the appropriate phenolic parent compound (if there are two phenol groups, it may be necessary to protect one of them using standard protection techniques for at least a part of the reaction), the ring system sulphamates according to the present invention are prepared essentially as follows. Likewise, a solution of the appropriate parent compound in anhydrous DMF is treated with sodium-hydride [60% dispersion; 1.2 equiv.] at 0° C. under an atmosphere of N₂. After evolution of hydrogen has ceased, sulfamoyl chloride in toluene [excess, ca. 5 equiv.] is added and the reaction mixture is poured into brine after warming to room temperature overnight and diluting with ethyl acetate. The organic fraction is washed exhaustively with brine, dried (MgSO₄), filtered and evaporated. The crude product obtained is purified by flash chromatography and recrystallisation to give the corresponding sulfamate. All the compounds are fully characterized by spectroscopic and combustion analysis.

The following compounds of the present invention are made and are found to be steroid sulphatase inhibitors in accordance with the present invention.

### EXAMPLE 10i

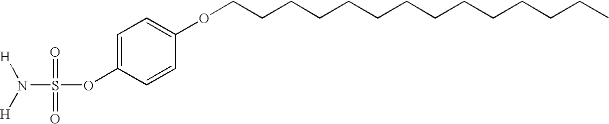

### EXAMPLE 10ii

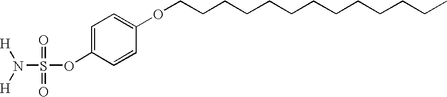

### EXAMPLE 10iii

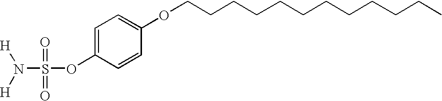

### EXAMPLE 10iv

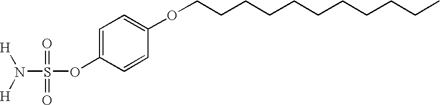

### EXAMPLE 10v

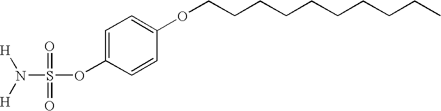

### EXAMPLE 10vi

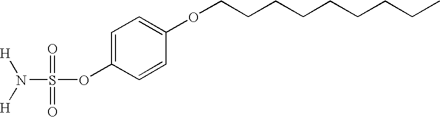

### EXAMPLE 10vii

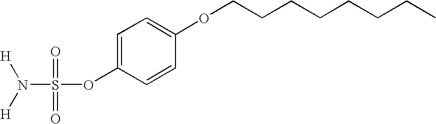

### EXAMPLE 10viii

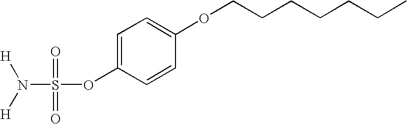

### EXAMPLE 10ix

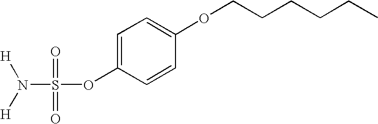

### EXAMPLE 10x

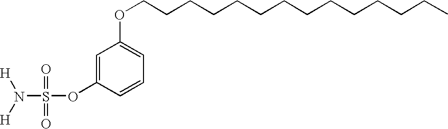

### EXAMPLE 10xi

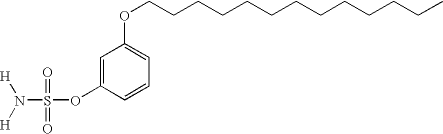

### EXAMPLE 10xii

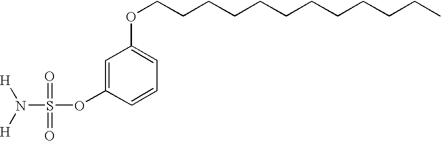

### EXAMPLE 10xiii

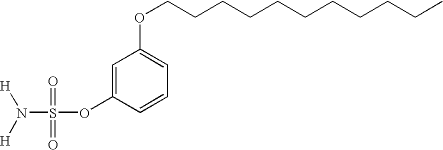

### EXAMPLE 10xiv

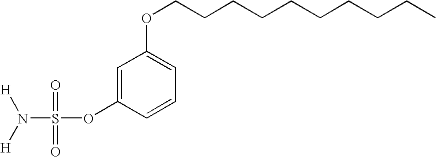

### EXAMPLE 10xv

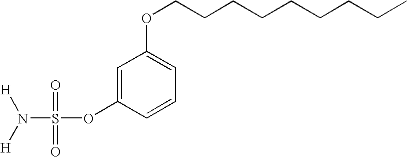

### EXAMPLE 10xvi

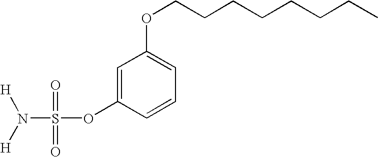

### EXAMPLE 10xvii

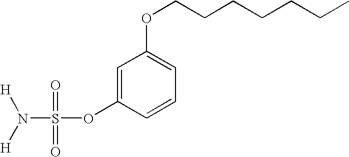

### EXAMPLE 10xviii

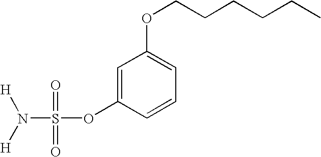

### EXAMPLE 10xix

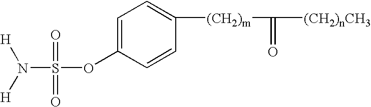 where n is an integer of from 1-3; and n is an integer of from 5-13.

### EXAMPLE 10xx

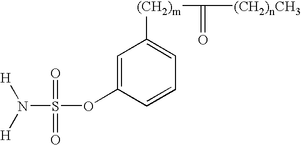 where n is an integer of from 1-3; and n is an integer of from 5-13.

### EXAMPLE 10xxi

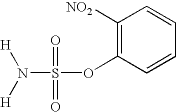

### EXAMPLE 10xxii

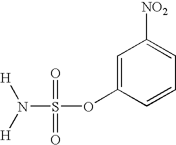

### EXAMPLE 10xxiii

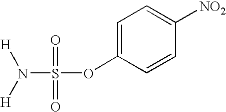

### EXAMPLE 10xxiv

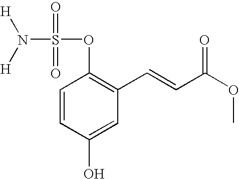

### EXAMPLE 10xxv

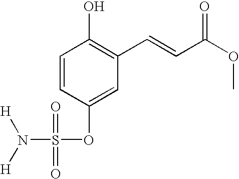

### EXAMPLE 10xxvi

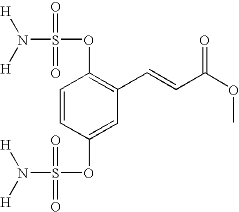

### EXAMPLE 10xxvii

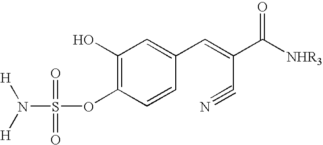 wherein R₃ is H or a suitable side chain—such as C₁₋₆ alkyl.

### EXAMPLE 10xxviii

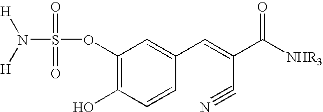 wherein R₃ is H or a suitable side chain—such as C₁₋₆ alkyl.

### EXAMPLE 10xxix

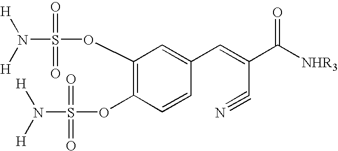 wherein R₃ is H or a suitable side chain—such as Clot alkyl.

### EXAMPLE 11

Starting with the appropriate phenolic parent compound (if there are two phenol groups, it may be necessary to protect one of them using standard protection techniques for at least a part of the reaction), the ring system sulphamates according to the present invention are prepared essentially as follows. Here, a solution of the appropriate parent compound in anhydrous DMF is treated with sodium hydride [60% dispersion; 1.2 equiv.] at 0° C. under an atmosphere of N₂. After evolution of hydrogen has ceased, N-methyl sulfamoyl chloride in toluene [excess, ca. 5 equiv.] is added and the reaction mixture is poured into brine after warming to room temperature overnight and diluting with ethyl acetate. The organic fraction is washed exhaustively with brine, dried (MgSO₄), filtered and evaporated. The crude product obtained is purified by flash chromatography and recrystallisation to give the corresponding sulfamate. All the compounds are fully characterized by spectroscopic and combustion analysis.

The following compounds of the present invention are made and are found to be steroid sulphatase inhibitors in accordance with the present invention. (In the following formulae, the methyl and H groups on the sulphamate group can be interchanged.)

### EXAMPLE 11i

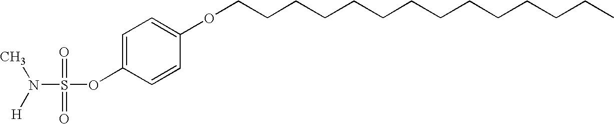

### EXAMPLE 11ii

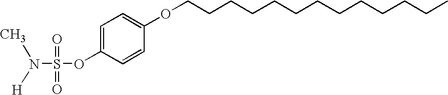

### EXAMPLE 11iii

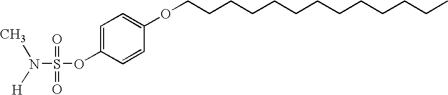

### EXAMPLE 11iv

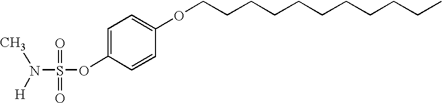

### EXAMPLE 11v

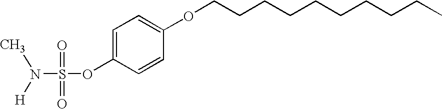

### EXAMPLE 11vi

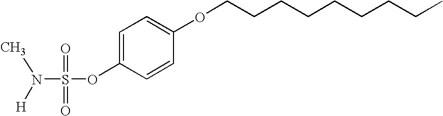

### EXAMPLE 11vii

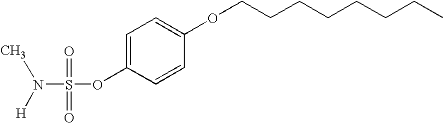

### EXAMPLE 11viii

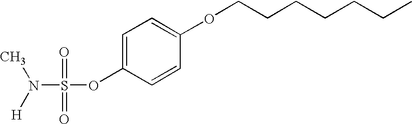

### EXAMPLE 11ix

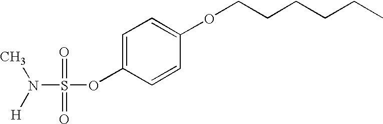

### EXAMPLE 11x

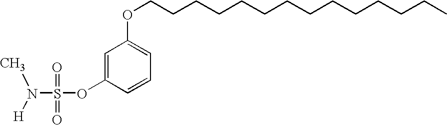

### EXAMPLE 11xi

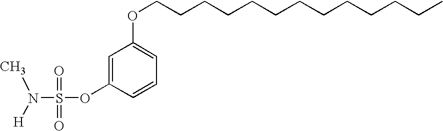

### EXAMPLE 11xii

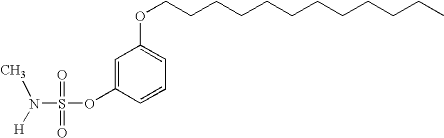

### EXAMPLE 11xiii

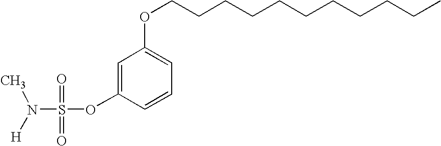

### EXAMPLE 11xiv

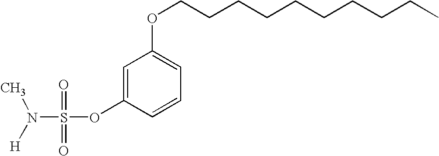

### EXAMPLE 11xv

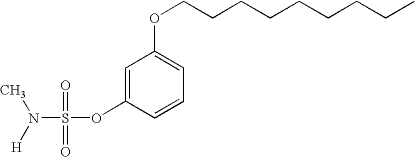

### EXAMPLE 11xvi

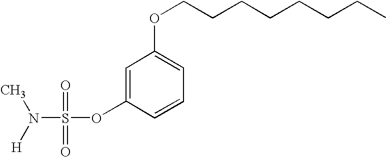

### EXAMPLE 11xvii

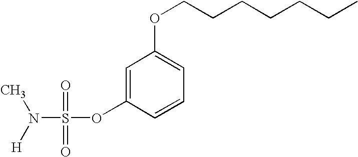

### EXAMPLE 11xviii

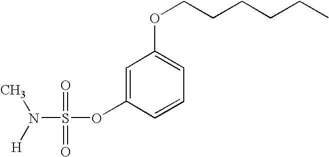

### EXAMPLE 11xix

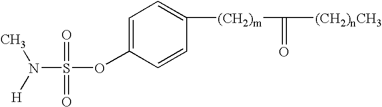 where n is an integer of from 1-3; and n is an integer of from 5-13.

### EXAMPLE 11xx

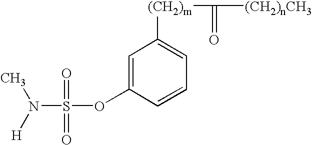 where n is an integer of from 1-3; and n is an integer of from 5-13.

### EXAMPLE 11xxi

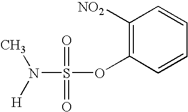

### EXAMPLE 11xxii

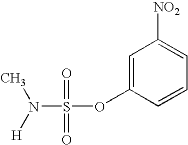

### EXAMPLE 11xxiii

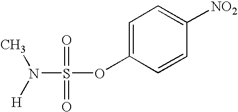

### EXAMPLE 11xxiv

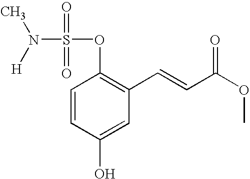

### EXAMPLE 11xxv

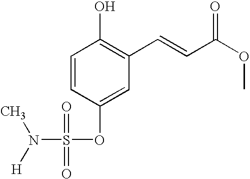

### EXAMPLE 11xxvi

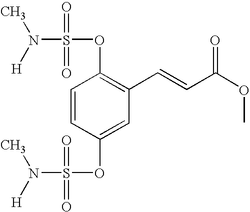

### EXAMPLE 11xxvii

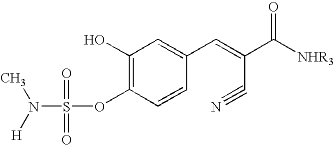 wherein R₃ is H or a suitable side chain—such as C₁₋₆ alkyl.

### EXAMPLE 11xxviii

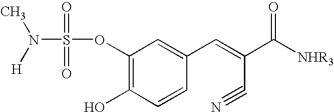 wherein R₃ is H or a suitable side chain—such as C₁₋₆ alkyl.

### EXAMPLE 11xxix

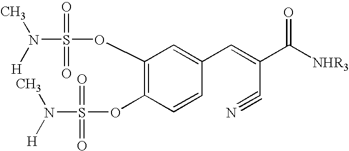 wherein R₃ is H or a suitable side chain—such as C₁₋₆ alkyl.

### EXAMPLE 12

Starting with the appropriate phenolic parent compound (if there are two phenol groups, it may be necessary to protect one of them using standard protection techniques for at least a part of the reaction), the ring system sulphamates according to the present invention are prepared essentially as follows. Here, a solution of the appropriate parent compound in anhydrous DMF is treated with sodium hydride [60% dispersion; 1.2 equiv.] at 0° C. under an atmosphere of N₂. After evolution of hydrogen has ceased, N, N-dimethyl sulfamoyl chloride in toluene [excess, ca. 5 equiv.] is added and the reaction mixture is poured into brine after warming to room temperature overnight and diluting with ethyl acetate. The organic fraction is washed exhaustively with brine, dried (MgSO₄), filtered and evaporated. The crude product obtained is purified by flash chromatography and recrystallisation to give the corresponding sulfamate. All the compounds are fully characterized by spectroscopic and combustion analysis.

The following compounds of the present invention are made and are found to be steroid sulphatase inhibitors in accordance with the present invention.

### EXAMPLE 12i

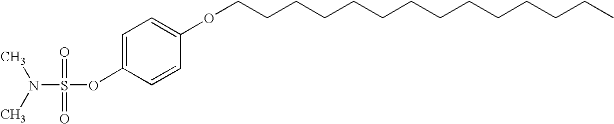

### EXAMPLE 12ii

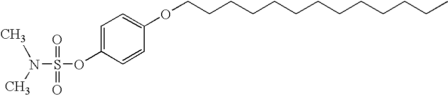

### EXAMPLE 12iii

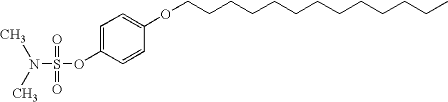

### EXAMPLE 12iv

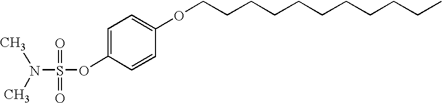

### EXAMPLE 12v

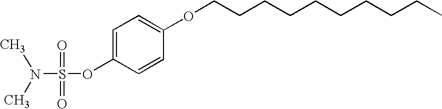

### EXAMPLE 12vi

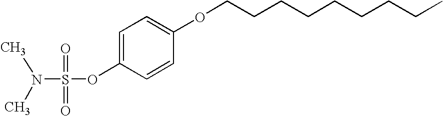

### EXAMPLE 12vii

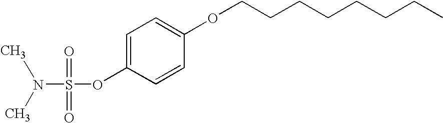

### EXAMPLE 12viii

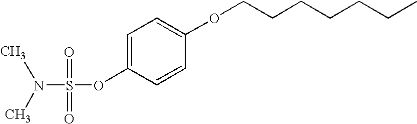

### EXAMPLE 12ix

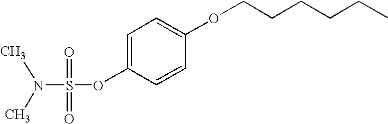

### EXAMPLE 12x

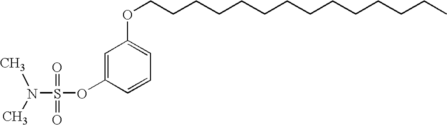

### EXAMPLE 12xi

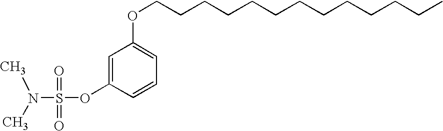

### EXAMPLE 12xii

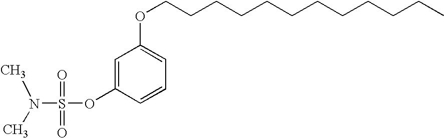

### EXAMPLE 12xiii

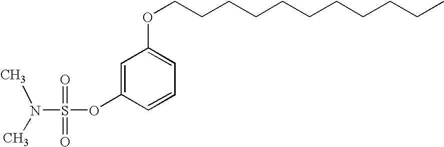

### EXAMPLE 12xiv

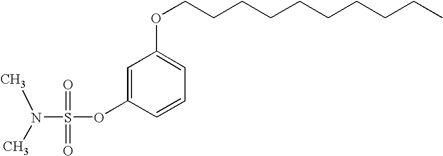

### EXAMPLE 12xv

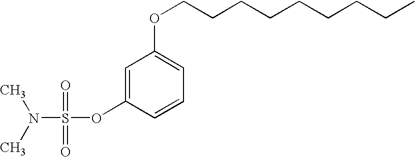

### EXAMPLE 12xvi

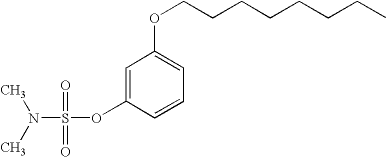

### EXAMPLE 12xvii

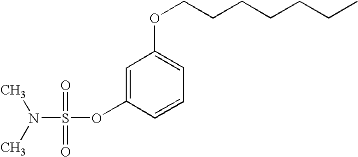

### EXAMPLE 12xviii

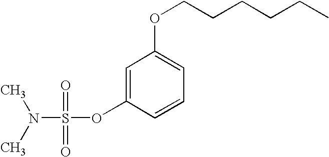

### EXAMPLE 12xix

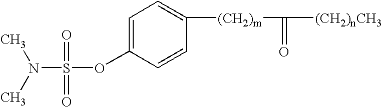 where n is an integer of from 1-3; and n is an integer of from 5-13.

### EXAMPLE 12xx

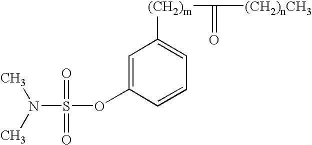 where n is an integer of from 1-3; and n is an integer of from 5-13.

### EXAMPLE 12xxi

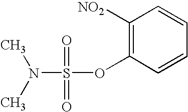

### EXAMPLE 12xxii

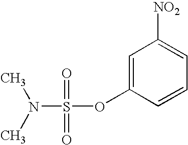

### EXAMPLE 12xxiii

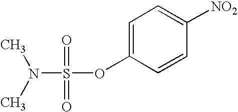

### EXAMPLE 12xxiv

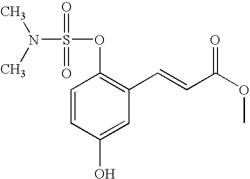

### EXAMPLE 12xxv

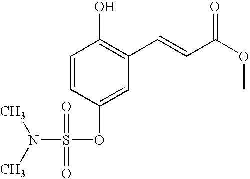

### EXAMPLE 12xxvi

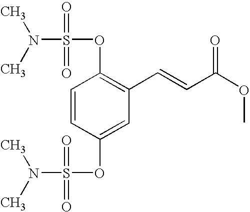

### EXAMPLE 12xxvii

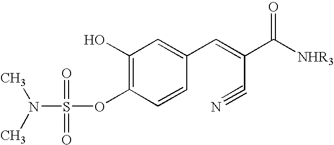 wherein R₃ is H or a suitable side chain—such as C₁₋₆ alkyl.

### EXAMPLE 12xxviii

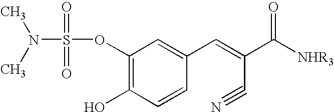 wherein R₃ is H or a suitable side chain—such as C₁₋₆ alkyl.

### EXAMPLE 12xxix

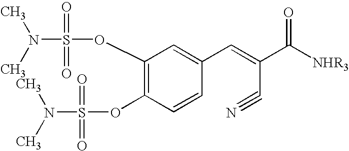 wherein R₃ is H or a suitable side chain—such as C₁₋₆ alkyl.

### EXAMPLE 13

The compounds of the present invention may be prepared by a process that comprises a Packman synthesis step. Packman synthesis is known in the art.

Sulphamoylation of Coumarins

The general procedure for the sulphamoylation of coumarins was as follows. A solution of an appropriate coumarin in anhydrous DMF (ca. 40 ml per g of coumarin) as treated with sodium hydride [60% dispersion; 1 equiv] at 0° C. under an atmosphere of N₂. After evolution of hydrogen had ceased, sulphamoyl chloride in toluene [ca. 0.68 M, 1.5 equiv] was added and the reaction mixture was poured into water after warming to room temperature overnight and then the crude product was then quenched. The organic fraction in ethyl acetate (150 ml) was washed exhaustively with brine, dried (MgSO₄), filtered and evaporated. The crude product obtained was purified by flash chromatography followed by recrystallisation to give the corresponding sulphamate. All new compounds were fully characterised by spectroscopic and combustion analysis. The synthesis of 4 methylcoumarin-7-O-sulphamate (14) is shown in FIG. 4.

Following this general procedure, compounds 13-16 (as shown in FIG. 2)—i.e. coumarin-7-O-sulphamate (13), 4-methylcoumarin-7-O-sulphamate (14), 3,4,8-trimethylcoumarin-7-O-sulphamate (15) and 4-(trifluoromethylcoumarin)-7-O-sulphamate (16)—were prepared. More details on the synthesis of these compounds now follow.

The synthesis of compound 12 (as shown in FIG. 2) is also discussed below.

Preparation of Coumarin-7-O-sulphamate (13)

Following the above-mentioned general procedure, 7-Hydroxycoumarin (500 mg, 3.082 mmol) gave a crude product (605 mg) which was fractionated on silica (200 g) by gradient elution with chloroform/acetone (8:1. 500 ml; 4:1, 1000 ml and then 2:1, 500 ml). Upon evaporation, the second fraction gave a creamy yellow residue (389 mg. 52.3%) which was recrystallised in ethyl acetate/hexane (1:1) to give (13) as dull white crystals (239 mg).

Analytical data were as follows:

M.p. 170.0-171.50° C.; R_{f}s=0.48 (ether), 0.67 (ethyl acetate), 0.19 (chloroform/acetone, 4:1); vmax (KBr) 3360, 3210, 3060, 1720, 1615, 1370. 1125 cm⁻¹; δ_{H} (DMSO-d₆/CDCl₃, ca. 1:25) 6.415 (1H, d, J_{C-4-H,C-3-H} =9.7 Hz, C-3-H), 7.285 (1H, dd, J_{C-8-H,C-6-H}=2.3 Hz and J_{C-5-H,C-6-H}+8.5 Hz, C-6-H), 7.379 (1H, d, J_{C-6-H,C-8-H}=2.2 Hz, C-8-H), 7.508 (2H, br s, D₂O exchanged, —NH₂), 7.543 (1H, d, J_{C-6-H,C-5-H}=8.4 Hz, C-5-H) and 7.760 (1H, d, J_{C-3-H,C-4-H}=9.7 Hz. C-4-H). MS: m/z (E.I., rel. intensity) 241.0(10), 162.0(97), 134.0(100), 105.0(23). Acc. MS: m/z 241.0068, C₉H₇NO₅S requires 241.0045. Found: C, 44.8; H, 2.89; N, 5.82. C₉H₇NO₅S requires C, 44.81; H. 2.92; N. 5.81%.

Preparation of 4-Methylcoumarin-7-O-sulphamate (14)

Following the above-mentioned general procedure, 7-Hydroxy-4-methylcoumarin(500 mg, 2.753 mmol) gave a crude product (633 mg) which was fractionated on silica (200 g) by gradient elution with chloroform/acetone (8:1. 500 ml; 4:1, 1000 ml, 2:1, 500 ml and then 1:1, 500 ml). Upon evaporation, the second fraction gave a creamy yellow residue (425 mg, 60.5%) which was recrystallised in acetone/chloroform (3:5) to give (14) as colorless rhombic crystals (281 mg).

Analytical data were as follows:

M.p. 165-167° C.; R_{f}s=0.48 (ether), 0.29 (ether/hexane 8:1). 0.26 (chloroform/acetone, 4:1); vmax (KBr) 3320, 3180, 3080, 1700, 1620, 1560, 1380, 1125 cm⁻¹; δ_{H} (acetone-d₆) 2.507 (3H, s, —CH₃) 6.339 (1H, s, C-3-H), 7.299 (2H, m, C-6-H and C-8-H), 7.390 (2H, br s, D₂O exchanged, —NH₂) and 7.850 (1H, d, J_{C-6-H,C-5-H}=9 Hz. C-5-H). MS: m/z (+ve ion FAB in m-NBA, rel. intensity) 542.2(15), 511.1[45, (2M+H)⁺], 461.2(20), 409.1[60, (M+H+NBA)⁺], 393.3[60, (M+H+NBA−16)⁺], 329.2[10, (M+H+NBA−80)⁺], 256.1 [100, (M+H)⁺]. MS: m/z (−ve ion FAB in m-NBA, rel. intensity) 421.0(20), 407.1[15, (M−H+NBA)⁻], 335.1(14), 254[100. (M−H)⁻], 175.1[32. (M−H−79)⁻], 121.0(17). Found: C, 47.2; H, 3.56; N, 5.51. C₁₀H₉NO₅S requires C, 47.06; H, 3.55; N, 5.49%.

Preparation of 3,4,8-Trimethylcoumarin-7-O-sulphamate (15)

Following the above-mentioned general procedure, 7-Hydroxy-3,4,8-trimethylcoumarin (1.0 g, 4.896 mmol) gave a crude product (1.33 g) which upon recrystallisation in hot ethyl acetate yielded 238 mg of starting coumarin. The mother liquor was evaporated and the white residue obtained (1.13 g) was fractionated on silica (200 g) with ether. The second fraction was collected, evaporated and the residue obtained (519 mg, 37.4%) was recrystallised in acetone/hexane (1:2) to give (15) as pale yellow crystals (312 mg).

Analytical data were as follows:

M.p. 197-202° C.; R_{f}s=0.50,(ether), 0.69 (ethyl acetate); vmax (KBr) 3310, 3040, 1680, 1600 cm⁻¹; δH (acetone-δ₆) 2.176. 2.383 and 2.458 (9H, three S, 3×CH₃), 7.374 (1H, d, J_{C-5-H,C-6-H}=8.8 Hz. C-6-H), 7.390 (2H, br s, D₂O exchanged, —NH₂) and 7.682 (1H, d, J_{C-6-H,C-5-H}=8.8 Hz, C-5-H). MS: m/z (E.I., rel. intensity) 283.1(10), 204.1(45), 176.1(40), 161.1(22), 69.1(56), 57.1(40), 43.1(100). Acc. MS: m/z 283.0497. C₁₂H₁₃NO₅S requires 283.0514. Found: C, 50.86; H, 4.63; N, 4.97. C₁₂H₁₃NO₅S requires C, 50.88; H, 4.63; N, 4.94%.

Preparation of 4-(Trifluoromethyl)coumarin-7-O-sulphamate (16)

Following the above-mentioned general procedure, 7-Hydroxy-4-(trifluoromethyl)-coumarin (0.90 g, 3.911 mmol) gave a crude product (1.20 g) which was fractionated on silica (200 g) with ether/chloroform (1:4). The residue (392 mg) from the third fraction was further purified by fractionating on silica (100 g) with ether. The first fraction then collected gave a residue (295 mg, 24.4%) which upon recrystallised in ethyl acetate/hexane (1:3) gave (16) as white needle-shaped crystals (160 mg).

Analytical data were as follows:

M.p. 165-168° C.; R_{f}s =0.67 (ether), 0.24 (ether/chloroform, 1:4); vmax (KBr) 3360, 3240, 3100, 1720, 1620, 1380, 1160 cm⁻¹; δ_{H} (acetone-d₆) 6.995 (1H, s, C-3-H), 7.461 (1H, dd, J_{C-8-H,C-6-H}=2.8 Hz and J_{C-8-H,C-6-H}=8.1 Hz, C-6-H), 7.478 (1H, s, C-8-H), 7.53 (2H, br s, D₂O exchanged, —NH₂) and 7.89 (1H, m, C-5-H), ¹H-NMR spectrum of (16) in DMSO-d₆/CDCl₃ (ca. 1:15) showed partial decompostion to the starting coumarin. MS: m/z (E.I., rel. intensity) 309.0(2.6), 230.0(77), 202.0(100), 183.5(5), 173.0(10), 69.0(33). Acc. MS: m/z 308.9874, C_{10H}₆F₃NO₅S requires 308.9919. Found: C, 38.8; H, 1.85; N, 4.53. C₁₀H₆F₃NO₅S requires C, 38.84; H, 1.96; N, 4.53%.

Preparation of 7-Sulphoxy)-4-Methylcoumarin, sodium salt (12)

To a solution of 7-hydroxy-4-methylcoumarin (1.0 g, 5.676 mmol) in dried pyridine (20 ml) under an atmosphere of N₂ [FIG. 8] was added sulphur trioxide-pyridine complex (1.8 g. 11.35 mmol, 2 equiv.) and the reaction mixture was stirred overnight. After removal of pyridine methanol (20 ml) was added to the creamy syrup obtained and the resulting light yellow solution was basified (pH ˜8) by dropwise addition of sodium hydroxide in methanol (1 M, ca. 18 ml). The bright yellow suspension formed was filtered and the precipitated washed with more methanol. The filtrate was then concentrated to 30-40 ml and ether (total 120 ml) was added in portions until precipitation completed. The light beige precipitate was collected (711 mg) and 582 mg of which was recrystallised in methanol/ether (1:1) to give (12) as light creamy yellow crystals (335 mg).

Analytical data were as follows:

M.p. 172-175° C. (dec.); R_{f}s=0.51 (methanol/ethyl acetate, 1:3), 0.67 (methanol/ether 1:3); vmax (KBr) 3500 (br), 3080, 1680, 1610, 1560, 1300, 1260, 1050 cm⁻¹: δ_{H} (DMSO-d₆) 2.407 (3H, s, —CH₃), 6.269 (1H, s, C-3-H), 7.20 (2H, m, C-6-H and C-8-H), and 7.695 (1H, d, J_{C-6-H,C-5-H}=8.8 Hz, C-5-H). MS: m/z (+ve ion FAB in m-NBA, rel. intensity) 176(100, NBA+Na⁺). MS: m/z (−ve ion FAB in m-NBA, rel. intensity) 175.1 (14, M−Na⁺−SO₃), 255.0 (100, M−Na⁺), 408.0 (8, M−Na⁺+NBA), 431.0 (15, M+153), 444.0(20), 533.0(15), 230.0(77), 202.0(100), 183.5(5), 173.0(10), 69.0(33). Acc. MS: m/z (−ve ion FAB in glycerol, rel. intensity) 254.9982(25), C₁₀H₇O₆S requires 254.9963. Found: C, 40.3; H, 2.92. C₁₀H₇O₆NaS.H₂O requires C, 40.55; H, 3.06%. HPLC [Spherisorb ODS5. 25×4.6 mm; Mobile phase: MeOH/H₂O (70:30), Flow rate: 1 ml/min; λₘₐₓ: 316 nm]: t_{R}=1.5 min. c.f. 7-hydroxy-4-methylcoumarin, 3.6 min.

Other data were as follows:

Compound 12 is stable in bases such as sodium hydroxide in methanol but not in acidic conditions. In addition, incomplete basification of the reaction mixture with sodium hydroxide in methanol (<3 equivalents) leads to decomposition of (12). Two equivalents of sodium hydroxide are required for consuming excess sulphur trioxide-pyridine complex to yield the neutral sodium sulphate. Insufficient amount of sodium hydroxide will therefore lead to the formation of sodium hydrogen sulphate which is acidic. Compound 12 appears labile to high temperature as one experiment has shown complete decomposition to 7-hydroxy-4-methylcoumarin after heating (12) as solid at 90° C. for 4 h.

In vitro tests

The above-mentioned coumarin sulphamates were tested for their ability to inhibit E1-STS activity using intact MCF-7 breast cancer cells or placental microsomes (100,000 g fraction) essentially as previously described.

To examine whether compound (12) could act as a substrate for E1-STS. 100 μg of the compound was incubated for 1 hour with placental microsomes in the absence or presence of EMATE (10 μM). The unconjugated coumarin formed at the end of the incubation was extracted with diethyl ether. After evaporation of solvent, the residue was examined by TLC using ethyl acetate/methanol (80:20) as eluent, in which the coumarin sulphate (12) and 7-hydroxy-4-methylcoumarin had R_{f} values of 0.79 and 0.95 respectively. Only unconjugated 7-hydroxy-4-methylcoumarin was detected after incubation of compound (12) with placental microsomes. The inclusion of EMATE in the reaction mixture reduced the hydrolysis of compound (12) by E1-STS, indicating that the coumarin sulphate is indeed a substrate for the sulphatase.

The dose-dependent inhibition of oestrone sulphatase in intact MCF-7 breast cancer cells by coumarin-7-O-sulphamate (13), 4-methylcoumarin-7-O-sulphamate (14), 3,4,8-trimethyl-coumarin-7-O-sulphamate (15) and 4-(trifluoromethyl)coumarin-7-O-sulphamate (16) can be seen from FIG. 5. Assays were performed essentially as previously described. Monolayers of intact MCF-7 cells in 25 cm³ flasks were incubated for 20 h at 37° C. with [³H]oestrone sulphate (2 nM) and coumarin sulphamates at 0.1-10 μM. Oestrone sulphatase activity was determined by measuring the total amount of ³H-labeled oestrone and oestradiol formed. Sulphatase activity in untreated cells was 100-200 fmol/20 h/10⁶ cells. Each point represents the mean ± s.d. of triplicate measurements.

The free parent coumarins of all coumarin sulphamates prepared showed little or no EI-STS inhibitory activity when tested up to 10 μM. However, in contrast, all four coumarin sulphamates (compounds 13-16) inhibited oestrone sulphatase inhibitory activity in a dose-dependent manner (FIG. 5) and the inhibition at 10 μM ranged from 71.5% for compound 16 to 93.3% for compound 14. The IC₅₀ for inhibition of E1-STS by compound 14, the most effective inhibitor, measured using intact MCF-7 cells was 380 nM.

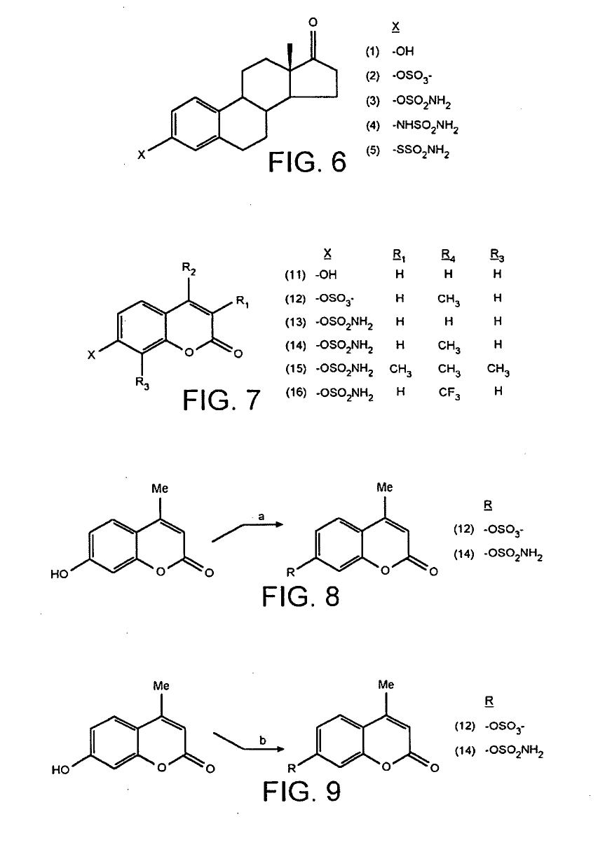

The time- and concentration-dependent inactivation of oestrone sulphatase by 4-methyl-coumarin-7-O-sulphamate (14) can be seen from FIG. 6. Placental microsomes (200 μg) were preincubated with (14) (control, ●; 0.5 μM, Δ and 10 μM, *) for 0-30 min at 37° C. followed by incubation with dextran-charcoal for 10 min at 4° C. Dextran-charcoal was sedimented by centrifugation and portions of the supernatants were then incubated with [³H]oestrone sulphate (20 μM) for 1 h at 37° C. to assess remaining sulphatase activity. Duplicate experiments were run at each concentration, but assays for residual activity were taken at different times in each experiment.

As with EMATE, compound 14 inhibited E1-STS activity in a time- and concentration-dependent manner in a biphasic fashion (FIG. 6), indicating a similar mechanism of action (potential chemical modification of two active site residues). At 10 μM, compound 14 reduced the original E1-STS activity by 95% after preincubating the enzyme with the inhibitor for 20 min.

Additional experiments revealed that compound 14 inhibited placental microsomal DHA-STS activity by 93.6% at the same concentration.

In Vivo Tests

In order to examine if compound 14 possessed oestrogenic activity and also to test its ability to inhibit E1-STS in vivo, it was administered to rats (1 mg/kg subcutaneously, in propylene glycol for 5 days) 14 days after ovariectomy had been performed.

Administration of compound 14 did not result in any significant increase in the uterine weight in these rats (data not shown), showing that compound 14 showed reduced oestrogenic agonist properties. The E1-STS activity in the uteri obtained from these animals was inhibited by 89.4% compared with. the activity in untreated animals.

Preliminary data also demonstrate potent oral activity in rats for compound 14, similar to that observed for EMATE.

In addition to these in vivo results, another series of rats (each weighing approximately 200 g) received 4-methyl coumarin-7-O-sulphamate (compound 14) orally in propylene glycol either as a single dose (SD) or daily for seven days (Multiple Dose, MD).

Inhibition of sulphatase activity was assessed in white blood cells (wbcs) that were collected after a SD or MD. Sulphatase activity was assayed using labelled oestrone sulphate as the substrate and measuring the release of oestrone.

The results are shown in FIG. 7 and in the Table below:

% Inhibition

| Dose mg/kg | SD | MD |
|---|---|---|
| 0.1 | 72 | 65 |
| 1.0 | 85 | 85 |
| 10.0 | 96 | 89 |

Similar results were found with liver cells.

Compound 14therefore demonstrates potent oral activity.

Other modifications of the present invention will be apparent to those skilled in the art. therapeutic agents that possess both aromatase and steroid sulphatase inhibitory properties.

Preferably, if the sulphamate group of the compound of the present invention were to be replaced with a sulphate group so as to form a sulphate compound then that sulphate compound would be hydrolysable by an enzyme having steroid sulphatase (E.C. 3.1.6.2) activity.

The compound of the present invention may have one or more sulphamate groups. For example, the compound may be a mono-sulphamate or a bis-sulphamate. For example, in FIGS. 7, 8, and 9 R₃ and R₄ may be each a sulphamate.

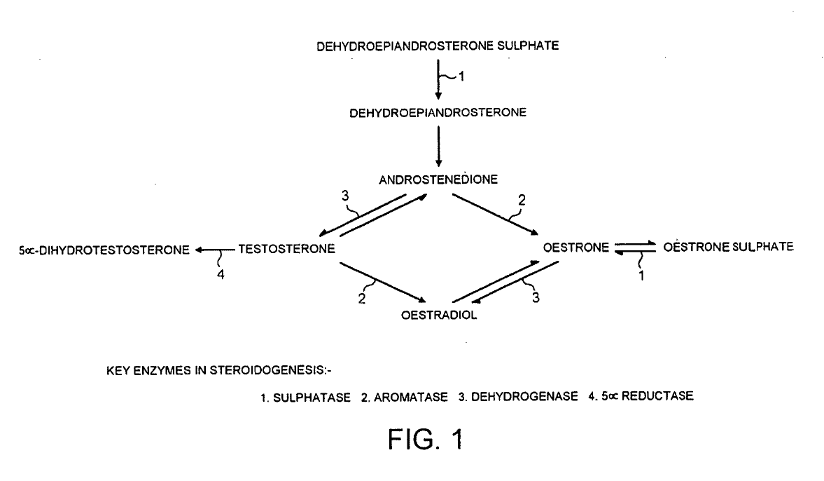

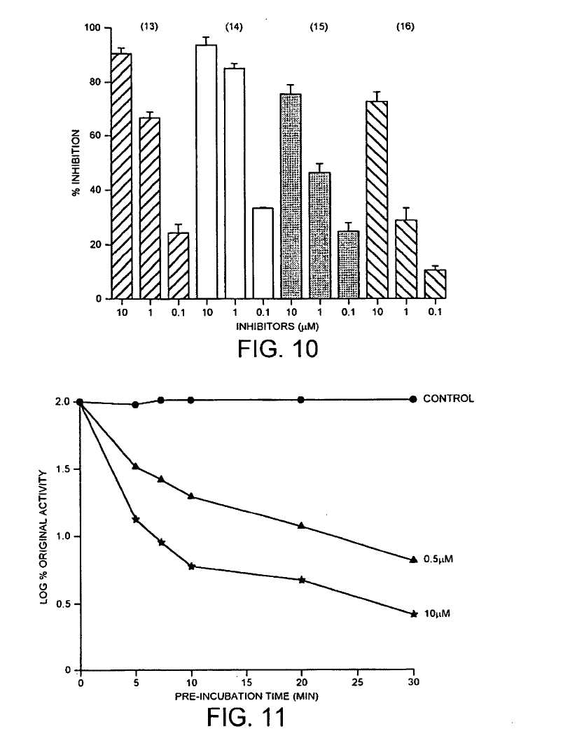

FIGS. 1 and 2 present schematic pathways; FIG. 3-10 present chemical formulae; and FIG. 11 presents a graph.

The present invention will now be described only by way of example.

Compounds Synthesised

The following sulphamate derivatives were synthesised from the following parent compounds:

| PARENT COMPOUND | SULPHAMATE COMPOUND |
|---|---|
| 1 | 2 |
| 3 | 4 |
| 5 | 6 |
| 7 | 8 |
| 9 | 10 |

wherein

1=6-hydroxy flavone

2=flavone-6-sulphamate

3=7-hydroxy flavone

4=flavone-7-sulphamate

5=5,7-dihydroxy flavone

6=5-hydroxy-flavone-7-sulphamate

7=5,7-dihydroxy-4′-hydroxy-flavone

8=5,7-dihydroxy flavanone-4′-flavanone sulphamate

9=5,7-dihydroxy-4′-methoxy-isoflavone

10=5-hydroxy-4′-methoxy-isoflavone-isoflavone-7-sulphamate

The formulae are presented in FIGS. 7-10

Synthesis

The sulphamate derivatives were prepared essentially as described previously. In this regard, a solution of the appropriate flavone, isoflavone or flavanone in anhydrous DMF was treated with sodium hydride (60% dispersion; 1 equiv for 2 and 4; 2 equiv for 6, 8 and 10) at 0° C. under an atmosphere of N₂. After evolution of hydrogen had ceased, sulfamoyl chloride (2 equiv except for 8, 5 equiv) was added and the reaction mixture was poured into brine after warning to room temperature overnight and diluting with ethyl acetate. The organic fraction was washed exhaustively with brine, dried (MgSO₄), filtered and evaporated. The crude product obtained was purified by flash chromatography and recrystallisation to give the corresponding sulfamate.

### Flavone 6-O-sulphamate (2)

6-Hydroxyflavone (1.0 g. 4.113 mmol) gave crude product (1.21 g) which was fractionated on silica (200 g) with ethyl acetate. Upon evaporation, the first fraction gave a creamy residue (760 mg, 58.2%) which was recrystallised in warm acetone/hexane (3:2) to give 2 as creamy rod-shaped crystals (557 mg). m.p. 190-191° C.; R_{f}s=0.71 (ethyl acetate), 0.51 (ethyl acetate/hexane, 2:1), vmax (KBr) 3260, 3040, 1620, 1600, 1580, 1370, 1180 cm⁻¹; δ_{H} (acetone-d₆) 6.917 (1H, s, C-3-H), 7.355 (2H, br s, exchanged with D₂O, —OSO₂NH₂), 7.64 (3H, m, C-3′-H, C-4′-H and C-5′-H), 7.75 (1H, dd, J_{C-8-H,C-7-H}=9 Hz and J_{C-5-H,C-7-H}=3 Hz, C-7-H), 7.87 (1H, d, J_{C-7-H,C-8-H}=9 Hz, C-8-H), 8.02 (1H, d, J_{C-7-H,C-5-H}=3 Hz, C-5-H) and 8.13 (2H, m, C-2′-H and C-6′-H), MS: m/z (E.I., rel. intensity) 317.0(11), 304.2(6), 238.0(96), 210.0(16), 187.1(14), 152.0(8), 136.0(100). Acc. MS (E.I.): m/z. 317.0296, C₁₅H₁₁NO₅S requires 317.0358. Found C, 56.7; H, 3.44; N, 4.31. C₁₅H₁₁NO₅S requires C, 56.78; H, 3.49; N, 4.41%.

### Flavone 7-O-sulphamate (4)

7-Hydroxyflavone (700 mg. 2.938 mmol) gave crude product (770 mg) which was fractionated on silica (200 g) with ethyl acetate. Upon evaporation, the first fraction gave a light brown residue (132 mg) which was recrystallised in hot isopropyl alcohol to give 4 as white needle-shaped crystals (60 mg), m.p. 172-174° C. (dec.); R_{f}s=0.78 (ethyl acetate), 0.56 (ethyl acetate/hexane, 4.1); vmax (KBr) 3260, 3100, 1630, 1600, 1400, 1580, 1200, 1150 cm⁻¹; δ_{H} (DMSO-d₆/CDCl₃, ca. 1:20) 6.824 (1H, s, C-3-H), 7.396 (1H, dd, J_{C-5-H,C-6-H}=8.8 Hz and J_{C-8-H,C-6-H}=2.2 Hz, C-6-H), 7.47 (2H, br s, exchanged with D₂O, —OSO₂NH₂), 7.55 (3H, m, C-;3′-H, C4′-H and C-5′-H), 7.639 (1H, d, J_{C-6-H,C-8-H}=2.2 Hz, C-8-H), 7.92 (2H, m, C-2′-H and C-6′-H) and 8.220 (1H, d, J_{C-6-H,C-5-H}=8.8 Hz. C-5-H). Found: C, 56.5: H, 3.36: N, 4.19. C₁₅H₁₁NO₅S requires C, 56.78; H, 3.49; N, 4.41%.

### 5-Hydroxyflavone 7-O-Sulphamate (6)

5,7-Dihydroxyflavone (1.0 g, 3.933 mmol) gave crude product (1.13 g) which was fractionated on silica (200 g) with chloroform/acetone (8:1). Upon evaporation, the second fraction gave a yellow residue (324 mg, 24.7%) which was recrystallised in ethyl acetate/hexane (1:1) to give 6 as yellow crystals (213 mg). m.p. 195-200° C. (dec.); R_{f}s =0.21, 0.25 and 0.44 for chloroform/acetone 12:1, 8:1 and 4:1

Respectively: vmax (KBr) 3360, 3250, 2925-2850, 1650, 1610, 1380 cm⁻¹, δ_{H} (acetone-d₆) 6.75, 6.98, 7.17 (3H, three s, C-3-H, C-6-H, C-8-H), 7.63 (2H, br s, exchanged with D₂O, —OSO₂NH₂), 7.65 (3H, m, C-3′-H, C-4′-H and C-5′-H), 8.15 (2H, d, J=7.7 Hz, C-2′-H and C-6′-H) and 13.0 (1H, br s, exchanged with D₂O, C-5-OH). MS: m/z (+ve ion FAB in m-NBA, rel, intensity) 440.1(10). 389.3(10). 334.1[100, (M+H)⁺, 288.1(17), 255.0[25, (M+H−79)⁺]. 169.1(30). MS: m/z (−ve ion FAB in m-NBA, rel. intensity) 499.0(30), 484.1[14, (M−2H+153)⁻, 475.1(20), 443.1(24), 332.1[100, (M−H)⁻], 308.1(28), 274.1(20), 253.1[50, (M−H−79)⁻], 195.1(24). Acc. MS (+ve ion FAB in m-NBA): m/z334.0392. C₁₅H₁₂NO₆S requires 334.0385. Found: C, 54.0; H, 3.39; N, 4.21. C₁₅H₁₁NO₆S requires C, 54.03; H, 3.33; N. 4.20%.

### 5,7-Dihydroxyflavanone 4′-O-sulphamate (8)

4′,5,7-Trihydroxyflavanone (1.0 g, 3.675 mmol) gave crude product (965 mg) which was fractionated on silica (200 g) with ethyl acetate/hexane (4:1) to give a mixture of the starting flavanone and product. This mixture was further fractionated on silica (200 g) with chloroform/acetone (4:1) and upon evaporation, the second fraction gave a pale yellow oil (345 mg, 34%) which solidified on standing. Subsequent recrystallisation of this solid in ethyl acetate/hexane (1:1) gave 8 as white crystals (259 mg). m.p. 211-213° C.; R_{f}=0.21 (chloroform/acetone, 4:1); vmax (KBr) 3420, 3340, 3260, 3140, 1640, 1510, 1380, 1160 cm⁻¹; δ_{H} (acetone-d₆) 2.84 (1H, dd, J_{AB}=17.4 Hz and J _{, eq}=3.1 Hz, C-3-H_{B}), 3.19 (1H, dd, J_{BA}=16.9 Hz and J _{,} =12.8 Hz, C-3-H_{A}), 5.62 (1H, dd, J _{, eq}=3.1 Hz and J _{,} =12.8 Hz. C-2-H), 5.98 (1H, d, J=2.0 Hz, C-6-H or C-8-H), 6.01 (1H, d, J=2.0 Hz, C-6-H or C-8-H), 7.20 (2H, br s, exchanged with D₂O, —OSO₂NH₂), 7.40 (2H, d, J=8.7 Hz. C-2′-H and C-6′-H), 7.66 (2H, d, J=8.7 Hz, C-3′-H and C-5′-H), 9.65 (1H, br s, C-7-OH) and 12.15 (1H, s, C-5-OH). MS: m/z (+ve ion FAB in m-NBA, rel. intensity) 352.0[100, (M+H)⁺], 288.1(10), 272.1[14. (M−79)⁻], 255.2(9),169.0(13). MS: m/z (−ve ion FAB in m-NBA, rel. intensity) 701.2(12), 606.2(10), 517.1(42), 504.1[20, (M+153)⁻, 473.2(10), 350.1(100, (M−H)⁻], 271.1(45, (M−H−79)⁻], 182.0(8). Acc. MS (+ve ion FAB in m-NBA): m/z 352.0496. C₁₅H₁₄NO₇S requires 352.0491. Found: C, 51.1; H, 3.68; N, 3.98. C₁₅H₁₃NO₇S requires C, 51.28: H, 3.73; N, 3.99%.

### 5-Hydroxy-4′-methoxyisoflavone 7′-O-sulphamate (10)

5,7-Dihydroxy-4′-methoxyisoflavone (800 mg, 2.817 mmol) gave crude product (650 mg) which was fractionated on silica (200 g) with chloroform/acetone (8:1). Upon evaporation, the second fraction gave a yellow residue (266 mg, 26%) which was recrystallised in ethyl acetate/hexane (1:1) to give 10 as yellow crystals (211 mg), m.p. 184-188° C.; R_{f}s=0.22 and 0.59 for chloroform/acetone 8:1 and 4:1 respectively; vmax (KBr) 3300-3020, 1660, 1610, 1400 cm⁻¹; δ_{H} (acetone-d₆) 3.86 (3H, s, —OCH₃), 6.75 (1H, d, J=2.2 Hz, C-6-H or C-8-H), 7.04 (3H m, C-6-H or C-8-H and C-3′-H and C-5′-H), 7.49 (2H, br s, exchanged with D₂O, —OSO₂NH₂), 7.58 (2H, d, J=7 Hz, C-2′-H and C-6′-H), 8.41 (1H, s, C-2-H), 13.05 (1H, br s, exchanged with D₂O, C-5-OH). MS: m/z (+ve ion FAB in m-NBA, ret. intensity) 393.3(12), 364.0[100, (M+H)⁺], 284.1[12, (M−79)⁺], 169.1(24), 134.0(22). MS: m/z (−ve ion FAB in m-NBA, ret. intensity) 529.1(25), 515.1[12, (M−H+153)⁻], 442.1(20), 362.1[100, (M−H)⁻], 308.1(34), 283.1[70, (M−H−79)⁻], 170.1(26). Acc. MS (+ve ion FAB in m-NBA): m/z 364.0494. C₁₆H₁₄NO₇S requires 364.0491. Found: C, 52.8; H, 3.65; N, 3.81. C₁₆H₁₃NO₇S requires C, 52.89; H, 3.61; N, 3.85%.

### 5-Hydroxy Isoflavone-4′,7-O,O-Disulphamate (11) and 5,7-Dihydroxy Isoflavone-4′-O-Sulphamate (12)

4′,5,7-Trihydroxy isoflavone (0.5 g, 1.85 mmol) upon sulphamoylation gave a crude product (0.65 g) which was fractionated on silica (200 g) with chloroform/acetone (4:1), and upon evaporation the third fraction gave a light yellow residue (0.329 g, 51%) which was recrystallizd in ethylacetate/hexane (1:2) to give compdound (11) as beige crystals (0.197 g); m.p=198° C. (dec); R_{f}s=0.14 and 0.24 for chloroform/acetone 4:1 and 2:1 respectively; v^{max} (KBr) 3460 (—NH₂), 1650 (C═O), 1400 (—SO₂N—) cm⁻¹; δ_{H} (acetone-d₆) 6.78 (1H, d, J=2.2 Hz, C-6-H or C-8-H, 7.03 (1H, d, J=2.2 Hz, C-8-H or C-6-H, 7.4 (4H, br s, exchanged with D₂O, C-4′-OSO₂NH₂ and C-7-OSO₂NH₂), 7.43 (2H, d, J=8.4 Hz, C-3′-H and C-5′-H or C-2′-H and C-6′-H and C-6′-H), 7.72 (2H, d, J=8.4 Hz, C-2′-H and C-6′-H or C-3′-H and C-5′-H), 8.51 (1H, s, C-2-H) and 12.93 (1H, s, C-5-OH). MS: m/z (+ve ion FAB in m-NBA, rel. intensity) 428.9 [100, (M+H)⁺, 350.0 [20, (M+H−SO₂NH₂)⁺], 272.1 [30, (M−H—SO₂NH₂)⁺]. MS: m/z (−ve ion FAB in m-NBA, rel. intensity) 426.9 [100, (M−H)⁻], 347.9 [95. (M−H—SO₂NH₂)⁻], 269.0 [30, (M−H—SO₂NH₂)⁻]. Acc. MS: m/z (FAB)⁺ 429.0083 C₁₅H₁₃N₂O₉S₂ requires 429.0063. Found C, 42.0; H, 2.91; N, 6.45; C₁₅H₁₂N₂O₉S₂ requires C, 42.06; H, 2.82; N, 6.54%.

The second fraction was collected and upon evaporation gave light yellow residue (0.112 g, 17%) which was recrystallized in ethylacetate/hexane (1:3) to give compound (12) as pale white crystals (0.068 g); m.p.=189-192° C. R_{f}s=0.23 and 0.33 for chloroform/acetone 4:1 and 2:1 respectively; v^{max} (KBr) 3500-3300 (—NH₂), 3200 (H-bonded-OH). 1680 (C═O), 1610, 1400 (—SO₂N-)cm⁻¹; δ_{H} (acetone-d₆) 6.32 (1H, d, J=2.2 Hz, C-6-H or C-8-H), 6.46 (1H, d, J=2.2 Hz, C-8-H or C-6-H), 7.32 (2H, br s, exchanged with D₂O, —O₂NH₂), 7.42 (2H, t, J=8.4 Hz, C-3′-H and C-5′-H or C-2′-H and C-6′-H, 7.69 (2H, d, J=8.4 Hz, C-2′-H and C-6′-H or C-3′-H and C-5′-H), 8.31 (1H, s, C-2-H), 9.53 (1H, s, C-7-OH) and 12.9 (1H, s, C-5-OH). MS: m/z (+ve ion FAB in m-NBA, rel. intensity) 350.0 [100, (M+H)⁺], 271.1 [15, (M+H—SO₂NH₂)⁺]. MS: m/z (−ve ion FAB in m-NBA, rel. intensity) 347.9 [100, (M−H)⁻], 269.0 [20, (M−H—SO₂NH₂)⁻]. Acc. MS: m/z (FAB)⁺ 350.0347 C₁₅H₁₂NO₇S requires 350.0335. Found C, 51.0; H, 3.16: N, 3.90; C₁₅H₁₁NO₇S requires C, 51.58; H, 3.17; N, 4.01%.

### Isoflavone-4′,7-O,O-Disulphamate (13)

4′,7-Dihydroxy isoflavone (0.45 g, 1.77 mmol) upon sulphamoylation gave a crude product (0.769 g) which was fractionated on silica (200 g) with chloroform/acetone (4:1), and upon evaporation the second fraction gave a pale white residue (0.553 g, 72%) which was recrystallized in acetone/hexane (1:2) to give the compound (13) as white crystals (0.327 g); m.p.>195° C. (dec.); R_{f}s=0.21 and 0.40 for chloroform/acetone 4:1 and 2:1 respectively; v^{max} (KBr) 3400 (—NH₂), 1640 (C═O), 1360 (—SO₂N—) cm⁻¹. δ_{H} (DMSO-d₆), 7.37 (2H, d, J=8.8 Hz, C-3′-H and C-5′-H or C-2′-H and C-6′-H, 7.42 (1H, dd, J_{C-6-H, C-5-H}=2.2 Hz, J_{C-6-H, C-5-H}=8.8 Hz, C-6-H), 7.7 (2H, d, J=8.8 Hz, C-2′-H and C-6′-H or C-3′-H and C-5′-H), 8.09 (2H, br s, exchanged with D₂O, —OSO₂NH₂), 8.24 (1H, d, J=8.8 Hz, C-5-H, 8.36 (2H, br s, exchanged with D₂O, —OSO₂NH₂), 8.63 (1H, s, C-2-H). MS: m/z (+ve ion FAB in m-NBA, rel. intensity) 412.9 [100, (M+H)⁺], 334.0 [25, (M+H—SO₂NH₂)⁺], 255.1 [20, (M+H—SO₂NH₂)⁺]. MS: m/z (−ve ion FAB in m-NBA, rel. intensity) 410.9 [100, (M−H)⁻], 332.0 (70, (M−H—SO₂NH₂)⁻], 253.0 [30, (M−H—SO₂NH₂)⁻]. Acc. MS: m/z (FAB)⁺ 413.0119 C₁₅H₁₃N₂O₈S₂ requires 413.0113. Found C, 44.0; H, 2.94; N, 6.62; C₁₅H₁₂N₂O₈S₂ requires C, 43.69; H, 2.93; N, 6.79%.

Assay of Inhibition of Sulphatase and Aromatase Activities

Sulphatase inhibition was assessed using placental microsome (100,000 g) preparations or intact MCF-7 breast cancer cells as described previously. Placental microsomes were incubated with ³H E1S, adjusted to 20 μM with unlabelled substrate, in the absence or presence of inhibitor.

Placental microsomes were also used to assess the aromatase inhibitory properties of the flavanoid sulphamates using a tritiated water release assay. Further placental microsomes (200 μl) were incubated with [1β-³H] androstenedione. 60 nM and 1 mM NADPH in the absence or presence of inhibitor.

Inhibition of Sulphatase and Aromatase Activities

Inhibition of oestrone sulphatase and aromatase activities in placental microsomes by the flavanoid sulphamate derivatives is shown in the Table below.

| | | % | % |
|---|---|---|---|
| | CONCENTRATION | INHIBITION | INHIBITION |
| COMPOUND | μM | Sulphatase | Aromatase |
| Flavone-6- | 1 | 26.8 | 1 |
| sulphamate | 10 | 89.5 | 6.5 |
| Flavone-7- | 1 | — | 55 |
| sulphamate | 10 | — | 86 |
| | 50 | 56.3 | |
| | 100 | 75.3 | |
| 5-hydroxy | 1 | 8 | 5 |
| flavone-7- | 10 | 21 | 76 |
| sulphamate | | | |
| 5,7-dihydroxy | 0.1 | 30.4 | Not tested |
| flavanone 4′- | 1 | 79.1 | Not tested |
| sulphamate | 10 | 98.1 | Not tested |
| 5-hydroxy-4′- | 1 | 1 | 2 |
| methoxy- | 10 | 50.6 | 5 |
| isoflavone-7- | | | |
| sulphamate | | | |

From the results, it can be seen that potent inhibition of sulphatase and aromatase activities was detected. For sulphatase inhibition this ranged from 21% at 10 μM by 5-hydroxy flavone-7-sulphamate, to 98% by 5,7-dihydroxy flavanone-4′-sulphamate at 10 μM. Potent aromatase inhibition was also achieved ranging from 6.5% by flavone-6-sulphamate at 10 μM to 86% by flavone-7-sulphamate at 10 μM.

Further In Vitro Testing

The following Table presents in vitro data for three isoflavones that were tested.

IN VITRO ACTIVITY

| | | % Inhibition | |
|---|---|---|---|
| | Concentration | MCF-7 | Placental |
| Compound | (μM) | Cells | Microsomes |
| Isoflavone 5-hydroxy- | 0.1 | 28 | nd |
| 4′,7-bissulphamate | 1.0 | 90 | nd |
| | 10.0 | 99 | 93 |
| Isoflavone 5,7-dihydroxy- | 0.1 | 23 | nd |
| 4′-sulphamate | 1.0 | 83 | nd |
| | 10.0 | 99 | 75 |
| Isoflavone-4′,7- | 0.1 | 89 | nd |
| bissulphamate | 1.0 | 99 | nd |
| | 10.0 | 99 | 99 |

nd = not done

In Vivo Testing

FIG. [symbol] presents in vivo inhibition of oestrone sulphatase activity in rat liver for two isoflavones according to the present invention. In this regard, BH22F1=5-hydroxy isoflavone-4′,7-bissulphamate; BH22BF1=5,7-dihydroxy isoflavone-4′-sulphamate. Compounds were administered as a single 10 mg/Kg dose. Oestrone sulphatase activity was assayed in tissue samples obtained 24 h after drug administration.

Other modifications of the present invention will be apparent to those skilled in the art.

Preparative Methods

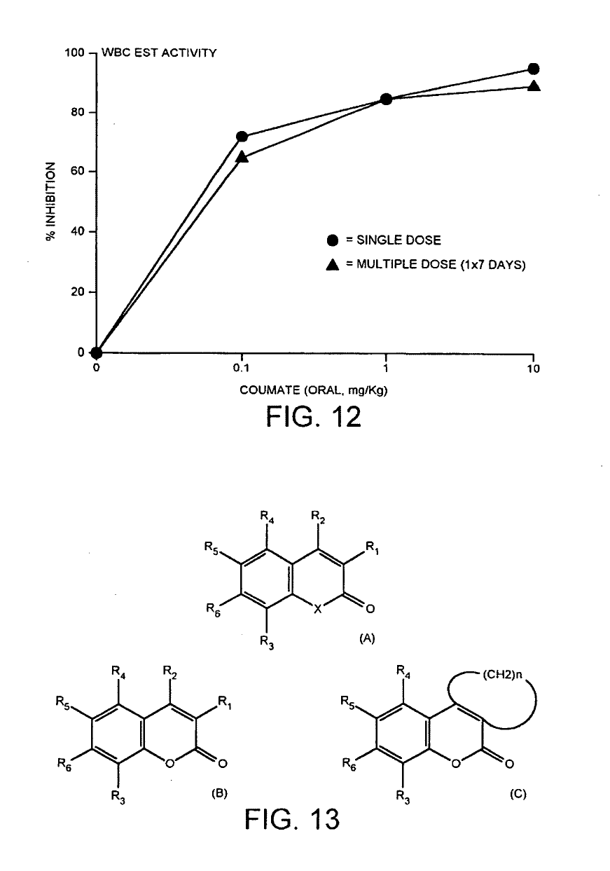

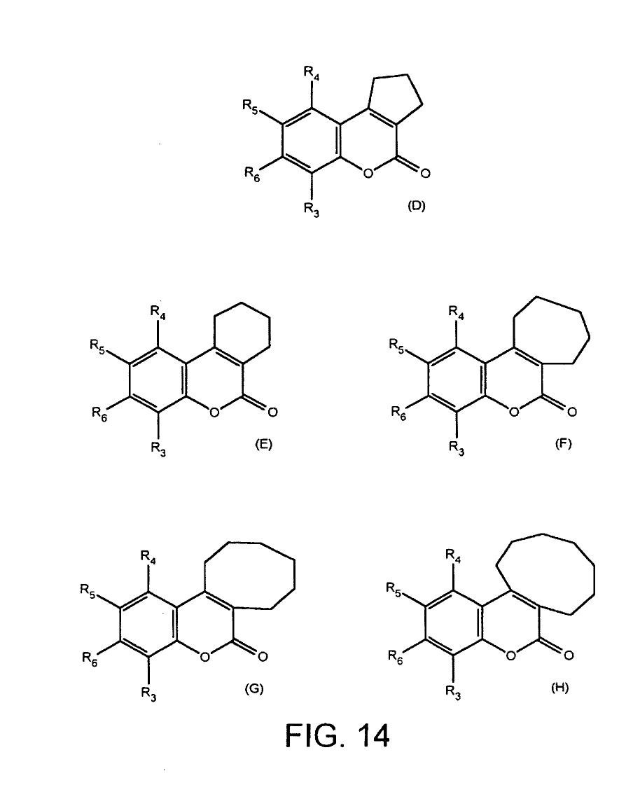

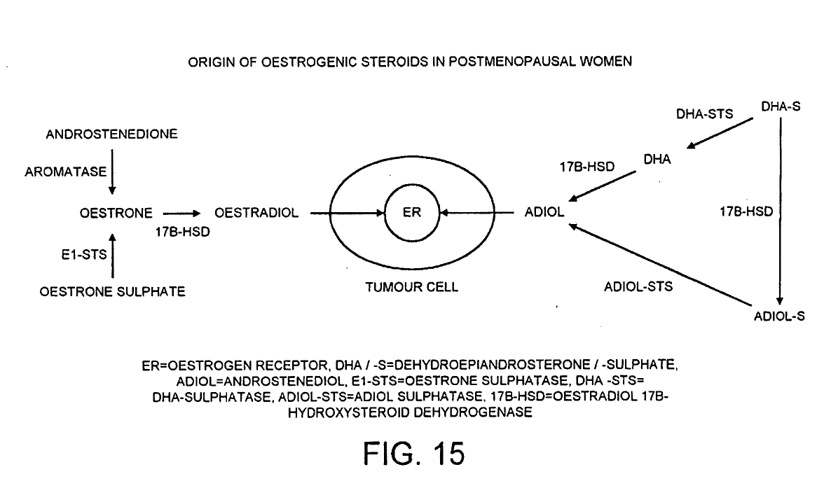

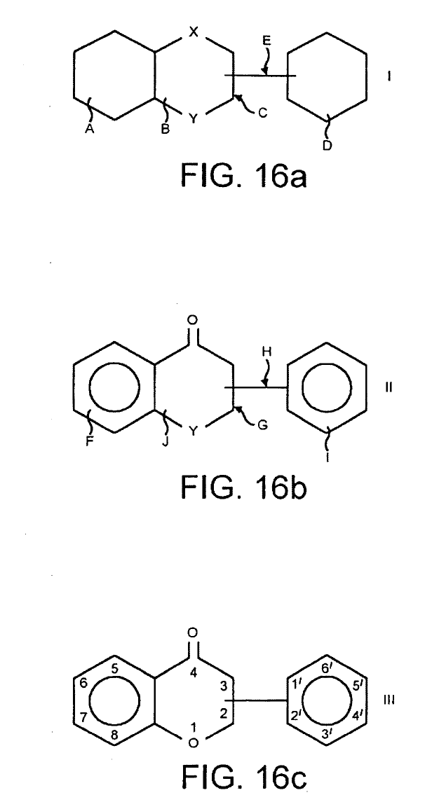

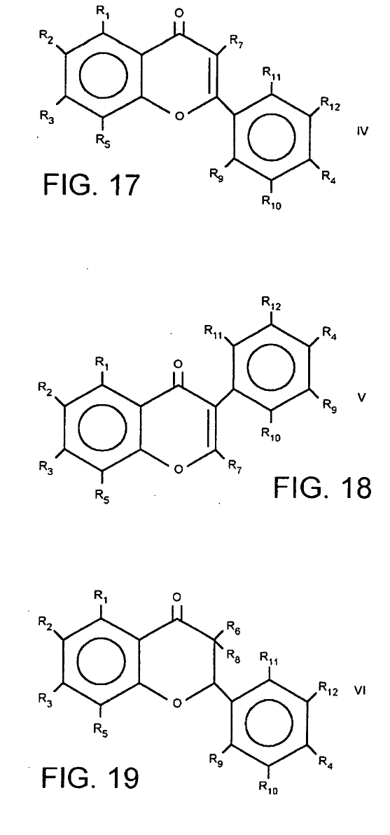

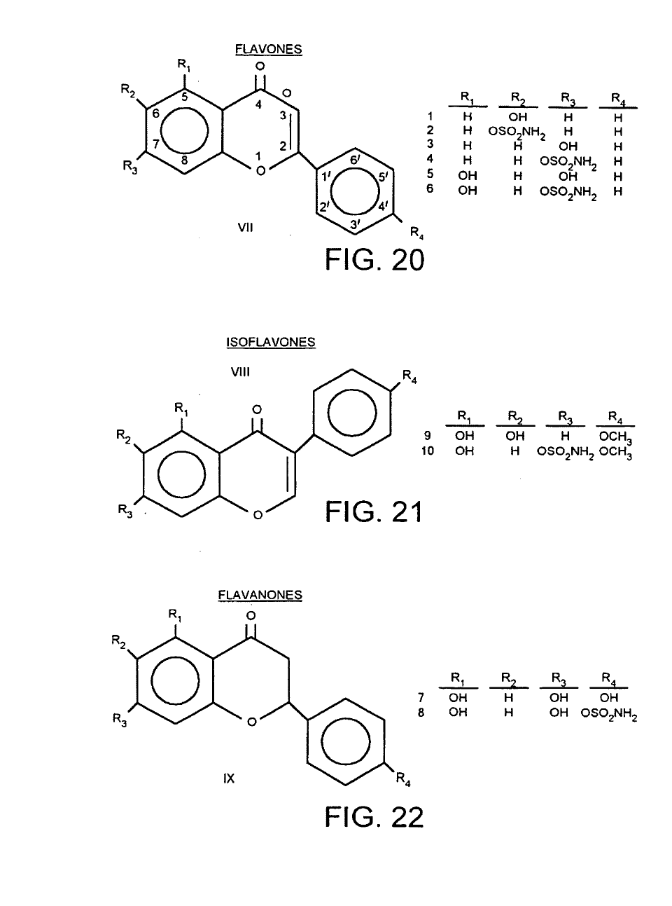

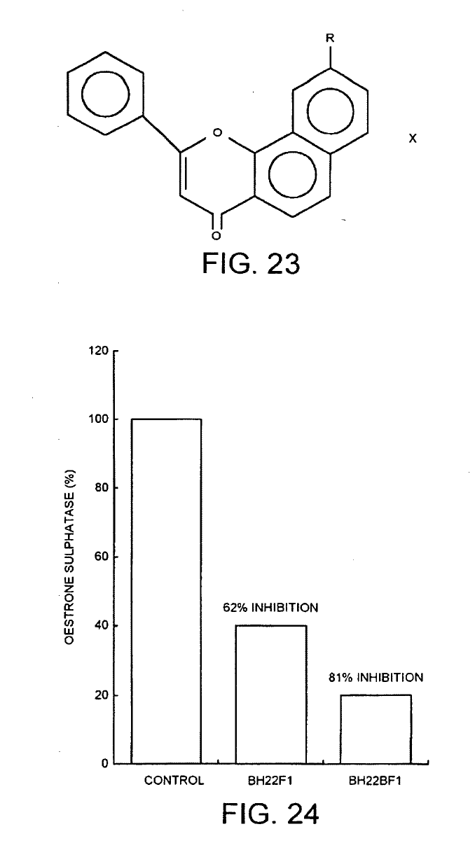

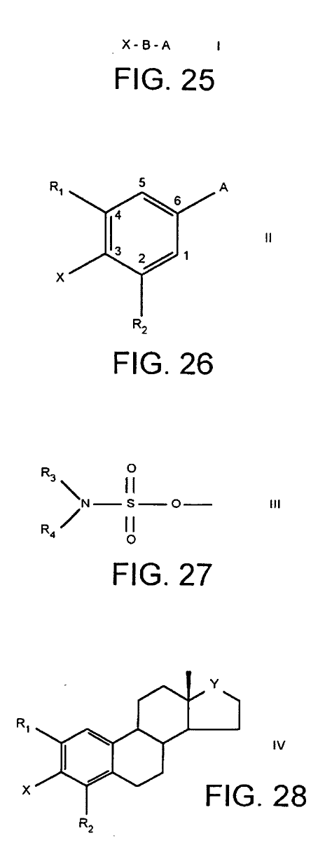

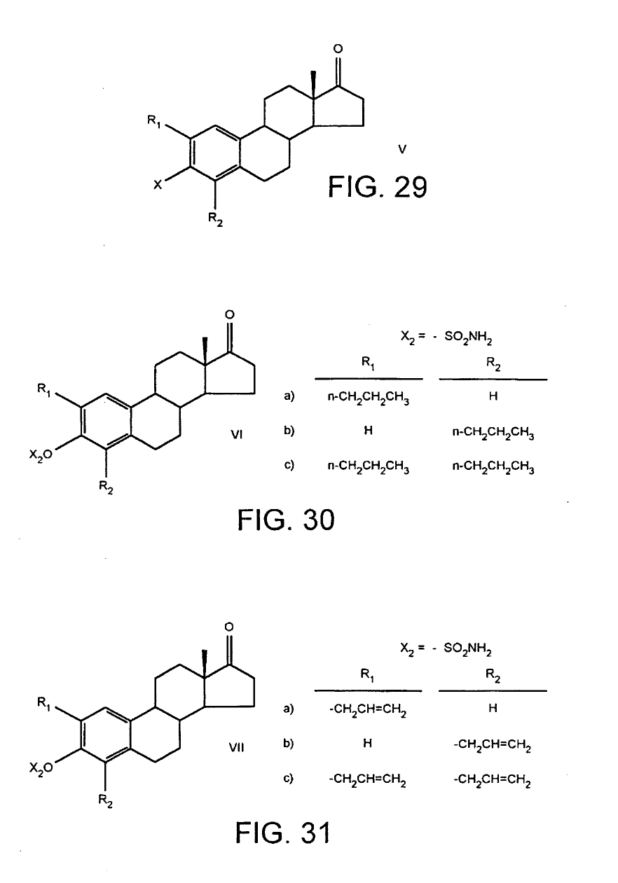

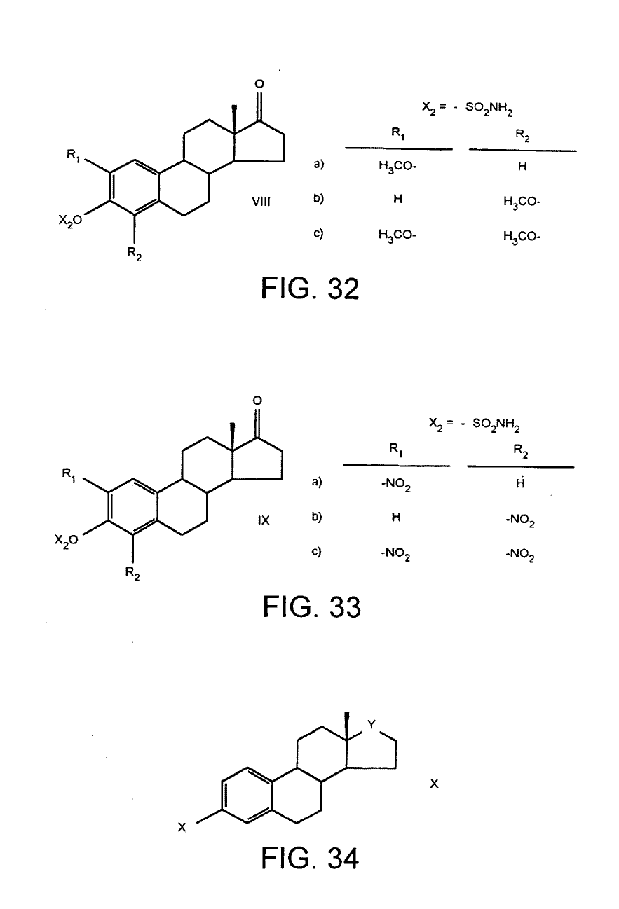

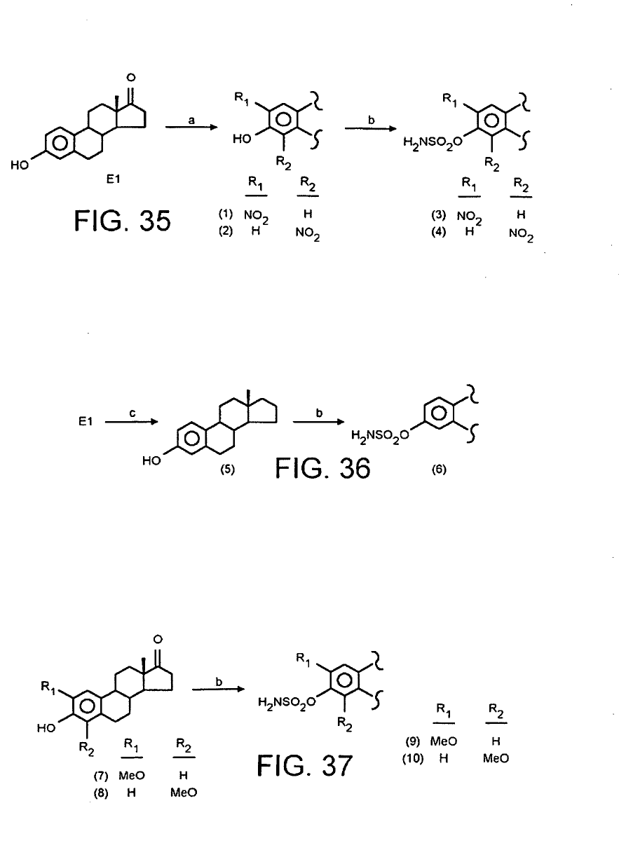

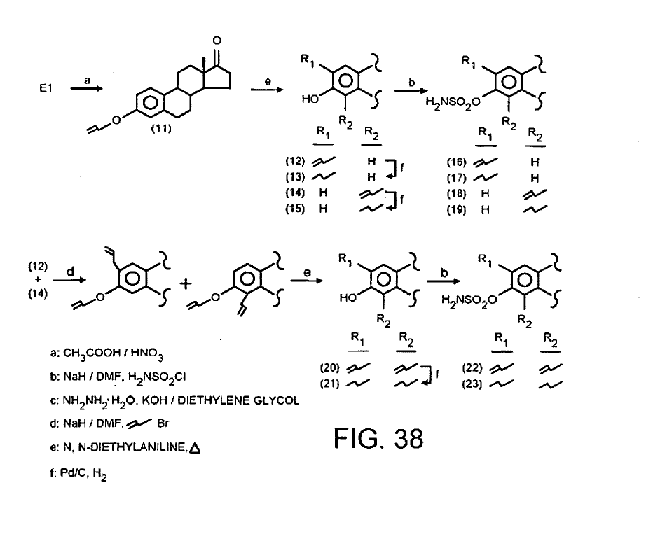

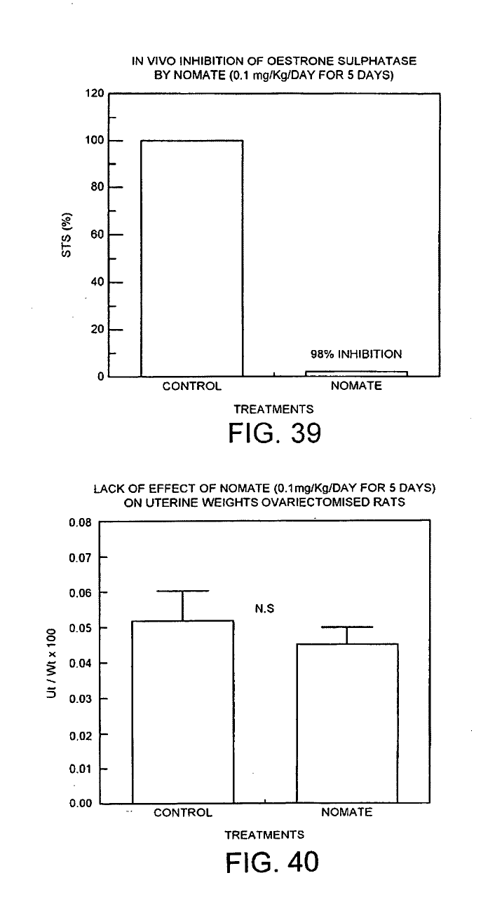

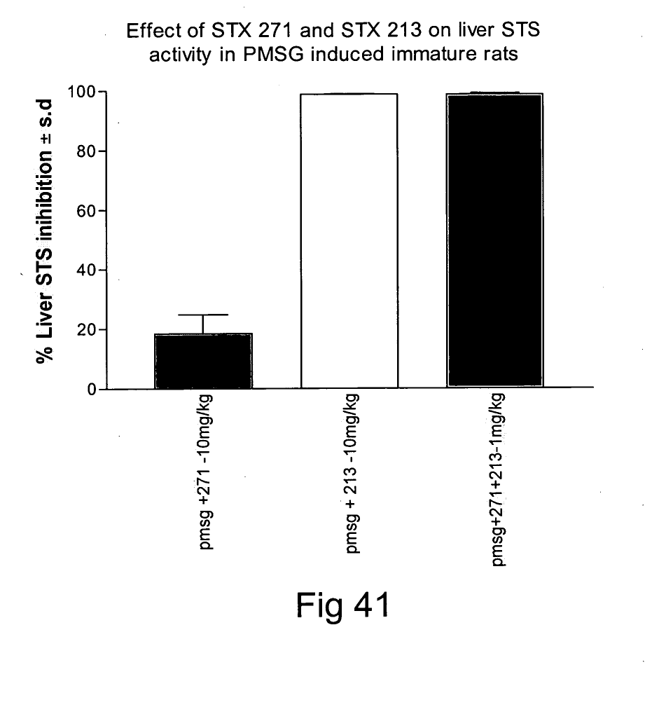

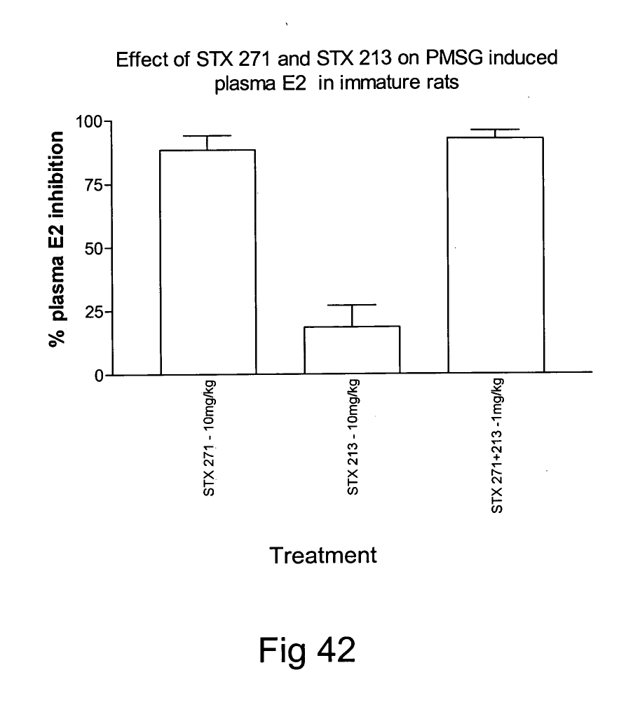

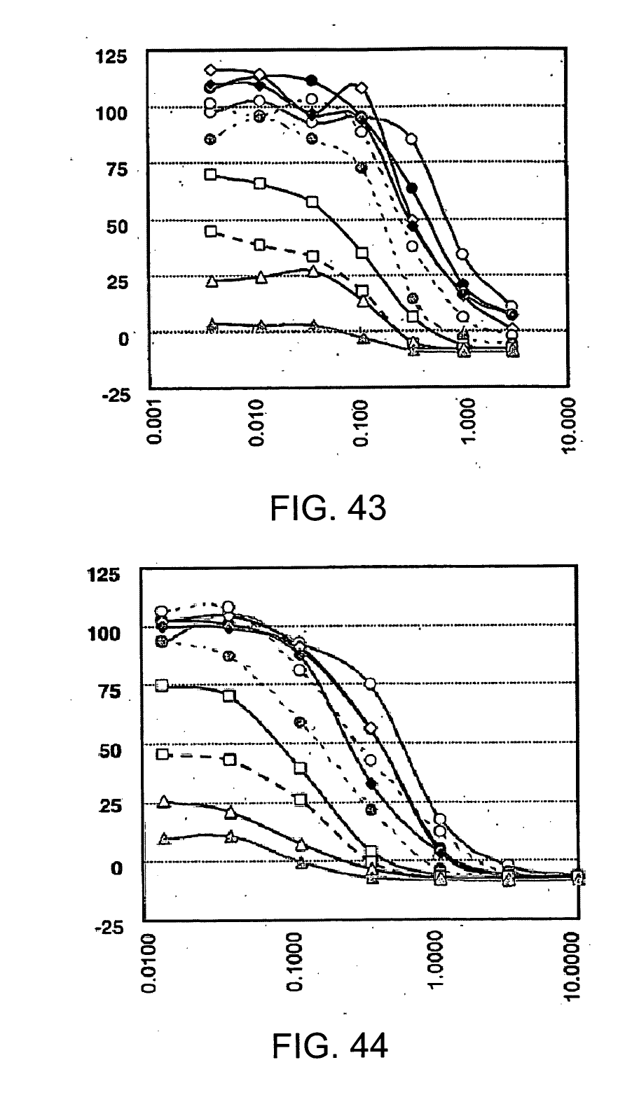

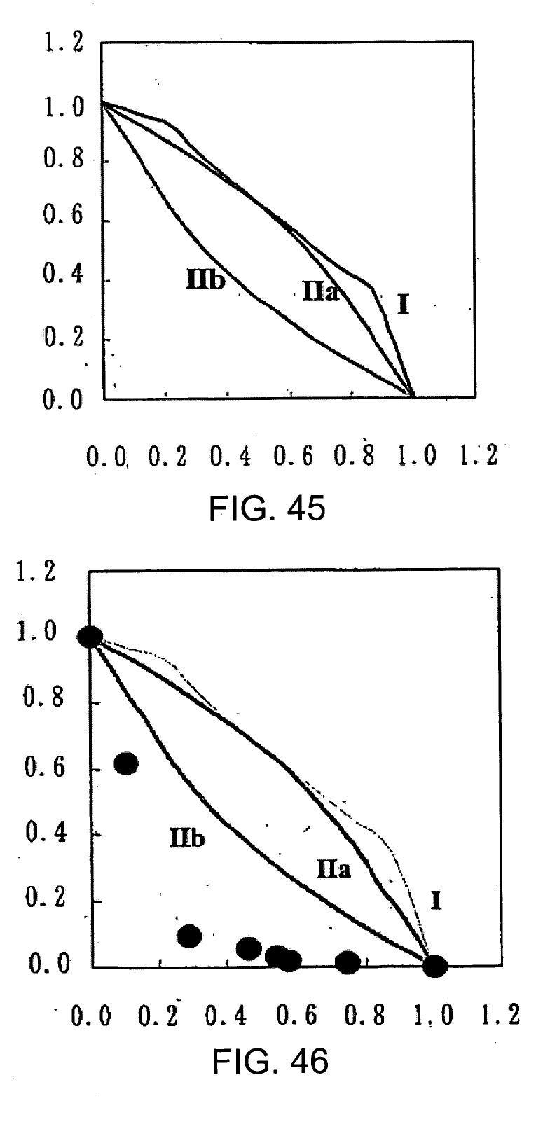

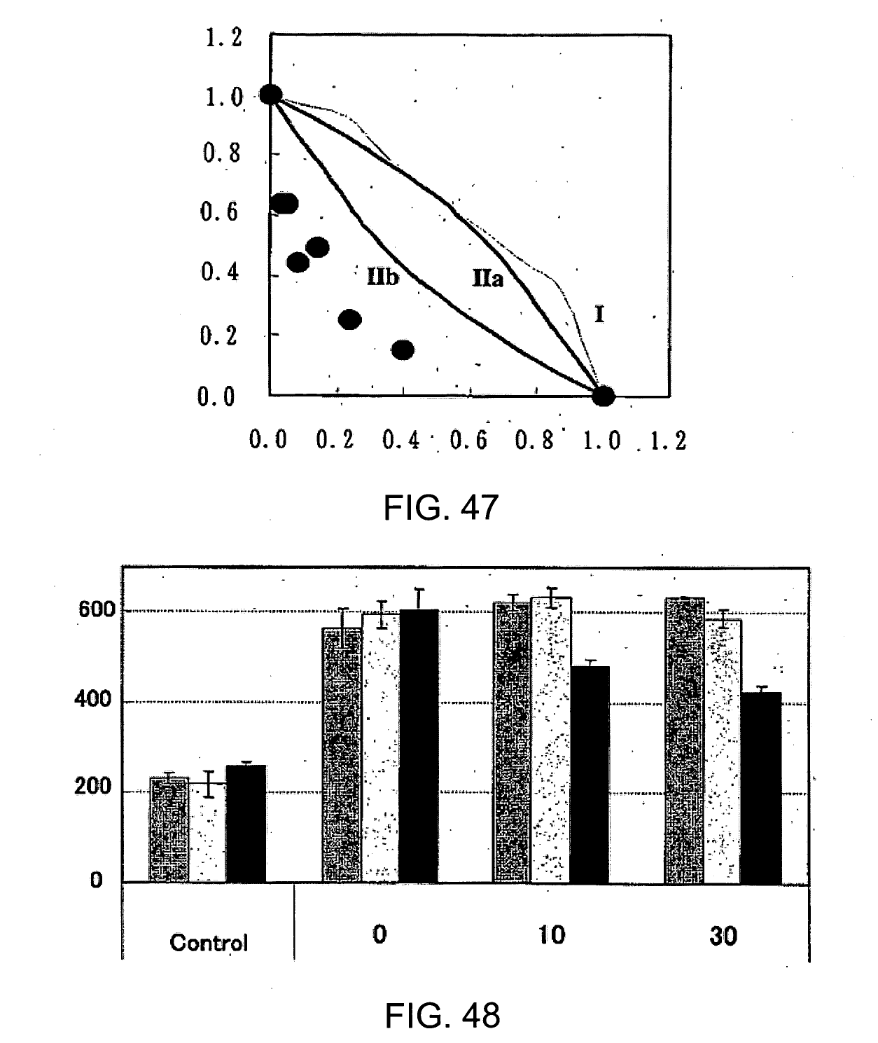

The preparation of various compounds in accordance with the present invention is illustrated in FIGS. 12 to 15. In these Figures, the curved lines attached to the phenyl rings represent the remainder of the ringed structure.

In Vitro Inhibition

The ability of compounds to inhibit oestrone sulphatase activity was assessed using either intact MCF-7 breast cancer cells or placental microsomes as previously described

In this regard, the teachings of that earlier reference are as follows:

Inhibition of Steroid Sulphatase Activity in MCF-7 cells by oestrone-3-sulphamate

Steroid sulphatase is defined as: Steryl Sulphatase EC 3.1.6.2.

Steroid sulphatase activity was measured in vitro using intact MCF-7 human breast cancer cells. This hormone dependent cell line is widely used to study the control of human breast cancer cell growth. It possesses significant steroid sulphatase activity (MacIndoe et al. Endocrinology, 123, 1281-1287 (1988); Purohit & Reed, Int. J. Cancer. 50, 901-905 (1992)) and is available in the U.S.A. from the American Type Culture Collection (ATCC) and in the U.K. (e.g. from The Imperial Cancer Research Fund). Cells were maintained in Minimal Essential Medium (Flow Laboratories, Irvine, Scotland) containing 20 mM HEPES, 5% foetal bovine serum, 2 mM glutamine, non-essential amino acids and 0.075% sodium bicarbonate. Up to 30 replicate 25 cm² tissue culture flasks were seeded with approximately 1×10⁵ cells/flask using the above medium. Cells were grown to 80% confluency and medium was changed every third day.

Intact monolayer of MCF-7 cells in triplicate 25 cm² tissue culture flasks were washed with Earle's Balanced Salt Solution (EBSS from ICN Flow, High Wycombe, U.K.) and incubated for 3-4 hours at 37° C. with 5 pmol (7×10⁵ dpm) [6,7-³H]oestrone-3-sulphate (specific activity 60 Ci/mmol from New England Nuclear, Boston, Mass., U.S.A.) in serum-free MEM (2.5 ml) together with oestrone-3-sulphamate (11 concentrations: 0; 1 fM, 0.01 pM; 0.1 pM; 1 pM; 0.01 nM; 0.1 nM; 1 nM; 0.01 mM; 0.1 mM; 1 mM). After incubation each flask was cooled and the medium (1 ml) was pipetted into separate tubes containing [¹⁴C]oestrone (7×10³ dpm) (specific activity 97 Ci/mmol from Amersham International Radiochemical Centre, Amersham, U.K.). The mixture was shaken thoroughly for 30 seconds with toluene (5 ml). Experiments showed that >90% [¹⁴C]oestrone and <0.1% [³H]oestrone-3-sulphate was removed from the aqueous phase by this treatment. A portion (2 ml) of the organic phase was removed, evaporated and the ³H and ¹⁴C content of the residue determined by scintillation spectrometry. The mass of oestrone-3-sulphate hydrolysed was calculated from the ³H counts obtained (corrected for the volumes of the medium and organic phase used, and for recovery of [¹⁴C]oestrone added) and the specific activity of the substrate. Each batch of experiments included incubations of microsomes prepared from a sulphatase-positive human placenta (positive control) and flasks without cells (to assess apparent non-enzymatic hydrolysis of the substrate). The number of cell nuclei per flask was determined using a Coulter Counter after treating the cell monolayers with Zaponin. One flask in each batch was used to assess cell membrane status and viability using the Trypan Blue exclusion method (Phillips, H. J. (1973) In: Tissue culture and applications, [eds: Kruse, D. F. & Patterson, M. K.]; pp. 406-408; Academic Press, New York).

Results for steroid sulphatase activity are expressed as the mean ±1 S.D. of the total product (oestrone+oestradiol) formed during the incubation period (20 hours) calculated for 10⁶ cells and, for values showing statistical significance, as a percentage reduction (inhibition) over incubations containing no oestrone-3-sulphamate. Unpaired Student's t-test was used to test the statistical significance of results.

Inhibition of Steroid Sulphatase Activity in Placental Microsomes by Oestrone-3-sulphamate

Sulphatase-positive human placenta from normal term pregnancies (Obstetric Ward, St. Mary's Hospital, London) were thoroughly minced with scissors and washed once with cold phosphate buffer (pH 7.4, 50 mM) then re-suspended in cold phosphate buffer (5 ml/g tissue). Homogenisation was accomplished with an Ultra-Turrax homogeniser, using three 10 second bursts separated by 2 minute cooling periods in ice. Nuclei and cell debris were removed by centrifuging (4° C.) at 2000 g for 30 minutes and portions (2 ml) of the supernatant were stored at −20° C. The protein concentration of the supernatants was determined by the method of Bradford (Anal. Biochem., 72, 248-254 (1976)).

Incubations (1 ml) were carried Out using a protein concentration of 100 mg/ml, substrate concentration of 20 mM [6,7-³H]oestrone-3-sulphate (specific activity 60 Ci/mmol from New England Nuclear, Boston, Mass., U.S.A.) and an incubation time of 20 minutes at 37° C. If necessary eight concentrations of compounds are employed: 0 (i.e. control); 0.05 mM; 0.1 mM; 0.2 mM; 0.4 mM; 0.6 mM; 0.8 mM; 1.0 mM. After incubation each sample was cooled and the medium (1 ml) was pipetted into separate tubes containing [¹⁴C]oestrone (7×10³ dpm) (specific activity 97 Ci/mmol from Amersham International Radiochemical Centre, Amersham, U.K.). The mixture was shaken thoroughly for 30 seconds with toluene (5 ml). Experiments showed that >90% [¹⁴C]oestrone and <0.1% [³H]oestrone-3-sulphate was removed from the aqueous phase by this treatment. A portion (2 ml) of the organic phase was removed, evaporated and the ³H and ¹⁴C content of the residue determined by scintillation spectrometry. The mass of oestrone-3-sulphate hydrolysed was calculated from the ³H counts obtained (corrected for the volumes of the medium and organic phase used, and for recovery of [¹⁴C]oestrone added) and the specific activity of the substrate.

For the present invention, the percentage inhibition for the series of EMATE analogues tested in either MCF-7 cells or placental microsomes is shown in Table 1

In Vivo Studies

Using 17-deoxy oestrone-3-O-sulphamate (NOMATE, FIG. [symbol] , Formula IV where X═—OSO₂NH₂, Y═—CH₂— and R₁ and R₂═H, and FIG. [symbol] as a representative example, the ability of this compound to inhibit oestrone sulphatase activity in vivo was examined in rats. The oestrogenicity of this compound was examined in ovariectomised rats. In this model compounds which are oestrogenic stimulate uterine growth.

i) Inhibition of oestrone sulphatase activity in vivo

NOMATE (0. 1 mg/Kg/day for five days) was administered orally to rats with another group of animals receiving vehicle only (propylene glycol). At the end of the study samples of liver tissue were obtained and oestrone sulphatase activity assayed using. ³H oestrone sulphate as the substrate as previously described.

As shown in FIG. 1, administration-of this dose of NOMATE effectively inhibited oestrone sulphatase activity by 98% compared widi untreated controls.

(ii) Lack of in vivo oestrogenicity

NOMATE (0.1 mg/Kg/day for five days) was administered orally to rats with another group of animals receiving vehicle only (propylene glycol). At the end of the study uteri were obtained and weighed with the results being expressed as uterine weight/whole body weight×100.

As shown in FIG. [symbol] , administration of NOMATE at the dose tested, but had no significant effect on uterine growth, showing that at this dose the compound is not oestrogenic.

TABLE 1
Inhibition of Oestrone Sulphatase Activity in MCF-7
Cells or Placental Microsomes by EMATE Analogues

| | | % Inhibition (Mean) | |
|---|---|---|---|
| | Concentration | MCF-7 | Placental |
| Inhibitor | Tested (mM) | Cells | Microsomes |
| 2-n-propyl EMATE | 0.1 | 41.1 | — |
| | 1 | 83.1 | 21.9 |
| | 10 | 92.2 | 43.2 |
| | 25 | — | 47.5 |
| | 50 | — | 61.1 |
| | 100 | — | 69.2 |
| 4-n-propyl EMATE | 1 | — | 13.7 |
| | 10 | — | 10.2 |
| | 25 | — | 15.7 |
| | 50 | — | 16.3 |
| | 100 | — | 23.7 |
| 2,4-n-dipropyl EMATE | 0.1 | 6.6 | — |
| | 1 | 10.6 | — |
| 2-allyl EMATE | 0.01 | 23.2 | — |
| | 0.1 | 76.1 | — |
| | 1 | 94.2 | 45.6 |
| | 10 | 93.7 | 65.4 |
| | 25 | — | 75.3 |
| | 50 | — | 86.6 |
| | 100 | — | 89.6 |
| 4-allyl EMATE | 1 | — | 29.1 |
| (approx 75%) | 10 | — | 54.2 |
| | 25 | — | 59.0 |
| | 50 | — | 65.1 |
| | 100 | — | 71.9 |
| 2,4-di-allyl EMATE | — | — | — |
| 2-methoxy EMATE | 0.1 | 96.0 | — |
| | 1 | 93.6 | — |
| | 10 | 96.2 | 99.0 |
| | 50 | — | 99.7 |
| | 100 | — | 99.7 |
| 2-nitro EMATE | 0.05 | — | 44.5 |
| | 0.5 | — | 93.9 |
| | 5 | — | 99.0 |
| | 50 | — | 99.4 |
| 4-nitro EMATE | 20 | — | 99.0 |
| NOMATE | 0.1 | 96.4 | 97.2 |
| (17-deoxy EMATE) | 1 | 99.1 | 99.5 |
| | 10 | 99.7 | 99.5 |
| | 25 | 99.7 | 99.7 |

— = not tested
Irreversible time- and concentration-dependent inhibition is assumed for these compounds in keeping with established precedent (Biochemistry, 1995, 34, 11508-11).

Other modifications of the present invention will be apparent to those skilled in the art.

### EXAMPLE 16

### In Vitro Studies of Effectiveness of Co-Administration of Aromatase Inhibitor and Steroid Sulphatase Inhibitor (AI+STSI)

These studies utilized:

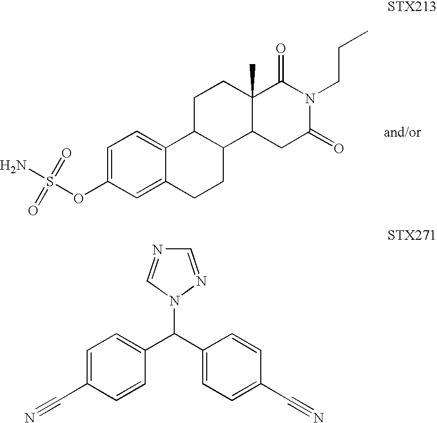

In vitro studies performed as described herein and/or as described in the literature demonstrated that co-administration of an AI+STSI completely blocked the activities of both enzymes.

An in vivo study performed as described herein and/or as described in the literature demonstrated that the co-administration of an AI+STSI blocks the activity of both enzymes.

The STSi data show (FIG. 41—Effect of STX271 and STX213 on liver STS activity in PMSG induced immature rats):
10 mg/kg of STX271 provide approximately 20% inhibition of STS
10 mg/kg of STX213 provides complete inhibition of STS
1 mg/kg of STX213 and 1 mg/kg of STX271 provide complete inhibition of STS

The AI data show (FIG. 42—Effect of STX271 and STX213 on PMSG induced plasma E2 in immature rats):
10 mg/kg of STX271 provide approximately 85% inhibition of aromatase
10 mg/kg of STX213 provide approximately 20% inhibition of aromatase
1 mg/kg of STX213 and 1 mg/kg of STX271 provide approximately 85% inhibition of aromatase.

### EXPERIMENTAL EXAMPLE 17

Proliferation inhibition of breast cancer cells using combination of antiestrogen agent and steroid-sulfatase inhibitor (With reference made to Experimental Example 1 of EP 1 568 381, which publication is not admitted to be prior art to the present application)

(1) MCS-2 cell in which human steroid-sulfatase is excessively expressed, is established from human breast cancer cell (MCF-7). Inhibition of the MCS-2 cell proliferation by an antiestrogen agent alone or a steroid-sulfatase inhibitor alone is compared with that by a combination of the steroid-sulfatase inhibitor and the antiestrogen agent. ICI-182780 is used as the antiestrogen agent, and Compound 17A is used as the steroid-sulfatase inhibitor.

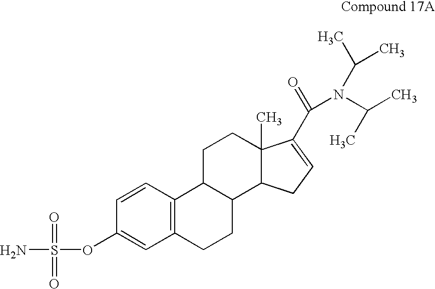

The MCS-2 cells are subcultured in a Phenol Red-free Eagle's minimum essential medium [PR(−)MEM; Nissui Pharmaceutical Co., Ltd.: referred to as medium A hereinafter] containing 5% bovine fetal serum (HyClone Laboratories Inc.) treated with dextran-charcoal, 1 mmol/L sodium pyruvate (Wako Pure Chemical Industries, Ltd.), 1% nonessential amino acid (NEAA; Dainippon Pharmaceutical Co., Ltd.), 2 mmol/L L-glutamine (GIBCO BRL), and 0.11% sodium hydrogencarbonate solution (ICN Biomedicals Inc.).

A dimethylsulfoxide (DMSO; Kanto Kagaku) solution containing 10 mmol/L estrone sulfate (Sigma Corp.) is diluted with medium A to a final concentration of 10⁻⁸ mol/L (medium B).

MCS-2 cells are diluted with the medium B containing estrone sulfate (final concentration: 10⁻⁸ mol/L) to a concentration of 2.5×10⁴ cells/mL and are inoculated in a 96-well microtiter plate (NUNC) in an amount of 100 mu L/well. The plate is incubated in an incubator set at 37 DEG C. and a humidity of 95% or more in a 5%-CO2 atmosphere for 24 hours, and then the medium is replaced with fresh estrone sulfate-containing medium B or fresh estrone sulfate-free medium A. To each well in which the medium is replaced with fresh estrone sulfate-containing medium B, test compound [(i) antiestrogen agent alone, (ii) a steroid-sulfatase inhibitor alone, or (iii) a combination of the antiestrogen agent and the steroid-sulfatase inhibitor: these agents are sequentially diluted with medium A] is added. To each well in which the medium is replaced with fresh estrone sulfate-free medium A, the agent is not added [(iv) agent-free]. The plate is incubated in an incubator set at 37 DEG C. and a humidity of 95% or more in a 5%-CO2 atmosphere for 168 hours. After the incubation, the supernatant is carefully removed such that all the cells are retained. Then, an MTT solution, which is prepared by dissolving 3-(4,5-dimethylthiazol-2-yl)-2,5-diphenyltetrazolium bromide (Sigma Corp.) in estrone sulfate-free medium A into a final concentration of 0.5 mg/mL, is added to each well in an amount of 50 mu L/well. The plate is incubated in an incubator set at 37 DEG C. in a 5%-CO2 atmosphere for 4 hours. After the MTT solution is removed, 0.1 mL of DMSO is added to each well. The plate is stirred with a plate mixer (Micro Mixer Model MX-4; Sanko Junyaku Co., Ltd.), and the formed formazan is eluted to measure the difference in absorbance at 550 nm and 630 nm with a plate reader (Spectra MAX 250; Wako Pure Chemical Industries, Ltd.). The results of inhibition of MCS-2 cell proliferation are indicated by a relative value of the number of MCS-2 cells in each condition to that in the agent-free condition, and are shown as a relative value (%) of each absorbance (MTT assay).

FIG. 43 shows inhibition curves on the MCS-2 cell proliferation under constant ICI-182780 concentrations while the concentration of Compound 17A is varied. FIG. 44 shows inhibition curves on the MCS-2 cell proliferation under constant Compound 17A concentrations while the concentration of ICI-182780 is varied.

As shown in FIG. 43, the inhibition of MCS-2 cell proliferation by Compound 17A is facilitated by the addition of ICI-182780 at a concentration of 0.234 nmol/L or more as compared with the results of the case ICI-182780 is not added.

As shown in FIG. 44, the inhibition of MCS-2 cell proliferation by ICI-182780 is facilitated by the addition of Compound 17A at a concentration of 0.140 nmol/L or more as compared with the results of the case Compound 17A is not added.

(2) In order to evaluate the efficacy of the combination treatment with the antiestrogen agent and the steroid-sulfatase inhibitor, an isobologram is constructed from the inhibition curves on the MCS-2 cell proliferation shown in FIGS. 43 and 44 according to International Journal of Radiation Oncology Biology Physics, page 85 (1979) and page 1145 (1979).

When the antiestrogen agent or the steroid-sulfatase inhibitor is used alone, the concentrations of each required to inhibit the proliferation of MCS-2 cells, i.e. the concentrations required to inhibit 50% of the proliferation (IC50 value), inhibit 45% of the proliferation (IC45 value), inhibit 40% of the proliferation (IC40 value), inhibit 35% of the proliferation (IC35 value), inhibit 30% of the proliferation (IC30 value), inhibit 25% of the proliferation (IC25 value), inhibit 20% of the proliferation (IC20 value), inhibit 15% of the proliferation (IC15 value), inhibit 10% of the proliferation (IC10 value), and inhibit 5% of the proliferation (IC5 value), are calculated from the inhibition curves on the MCS-2 cell proliferation shown in FIGS. 43 and 44 in the above-mentioned (1) with measurement software (the equation used in Soft Max Pro) equipped with a plate reader. An isobologram for IC50 values is constructed using these IC5 to IC50 values according to the above-mentioned paper. The isobologram is shown in FIG. 45.

(3) In order to evaluate the efficacy of the combination treatment, each concentration of the agents showing IC50 values in combination is plotted on the isobologram shown in FIG. 45 according to a method described in International Journal of Radiation Oncology Biology Physics, page 85 (1979) and page 1145 (1979).

IC50 values under the conditions where ICI-182780 concentrations were constant, i.e. concentrations of Compound 17A when 50% of the proliferation of MCS-2 cells is inhibited, are plotted on the isobologram (FIG. 45) constructed in the above-mentioned (2). The results are shown in FIG. 46. IC50 values under the conditions where Compound 17A concentrations are constant, i.e. concentrations of ICI-182780 when 50% of the proliferation of MCS-2 cells is inhibited, are plotted on the isobologram (FIG. 45) constructed in the above-mentioned (2). The results are shown in FIG. 47.

When the plots lay below a line of mode I on the isobologram, it is determined that the combination treatment had an additive effect. When the plots lay below a line of mode IIa, it is determined that the combination treatment further had a supra-additive effect.

As shown in FIG. 46, when ICI-182780 is used at constant concentrations, the addition of Compound 17A supra-additively inhibited the cell proliferation.

As shown in FIG. 47, when Compound 17A is used at constant concentrations, the addition of ICI-182780 supra-additively inhibited the cell proliferation.

### EXPERIMENTAL EXAMPLE 18

Proliferation inhibition of breast cancer cells using combination of aromatase inhibitor and steroid-sulfatase inhibitor (With reference made to Experimental Example 2 of EP 1 568 381, which publication is not admitted to be prior art to the present application)

Proliferation inhibition of breast cancer cell line (MCS-2) excessively expressing a human steroid-sulfatase by using an aromatase inhibitor alone or a steroid-sulfatase inhibitor alone is compared with that using a combination of the steroid-sulfatase inhibitor and the aromatase inhibitor. Vorozole is used as the aromatase inhibitor, and Compound 17A is used as the steroid-sulfatase inhibitor.

Medium C containing 10⁻⁸ mol/L estrone sulfate (final concentration) and 10⁻⁷ mol/L testosterone (final concentration) is prepared by diluting a DMSO solution containing 10 mmol/L estrone sulfate (Sigma Corp.) and a DMSO solution containing 10 mmol/L testosterone (Sigma Corp.) with medium A as described in Experimental Example 1.

MCS-2 cells are diluted with medium A to 2.5×10⁴ cells/mL, and then are inoculated in a 24-well microtiter plate (NUNC) at an amount of 100 mu L/well. The plate is incubated in an incubator set at 37 DEG C. and a humidity of 95% or more in a 5%-CO2 atmosphere for 24 hours, and then the medium is replaced with fresh medium C or fresh medium A. Medium C contains estrone sulfate at a final concentration of 10⁻⁸ mol/L and testosterone at a final concentration of 10⁻⁷ mol/L. Medium A contains neither estrone sulfate nor testosterone. To each well in which the medium is replaced with medium C, test compound diluted with a medium A [(i) an aromatase inhibitor alone, (ii) a steroid-sulfatase inhibitor alone, or (iii) a combination of the aromatase inhibitor and the steroid-sulfatase inhibitor] is added, or is not added [(iv) agent-free]. The plate is incubated in an incubator set at 37 DEG C. and a humidity of 95% or more in a 5%-CO2 atmosphere for 168 hours. The test compound are not added to the wells in which the medium is replaced with medium A (which contains neither estrone sulfate nor testosterone). These wells are incubated under the same conditions as above and used as controls. After the incubation, the supernatant is carefully removed such that all the cells are retained. Then, the wells are carefully rinsed with a phosphate buffer (GIBCO) such that all the cells are retained. A solution of 0.25% trypsin (GIBCO) and an aqueous solution of 0.02% ethylenediaminetetraacetic acid (EDTA, Wako Pure Chemical Industries, Ltd.) are added to the wells to thoroughly suspend the cells. The number of cells in each well is counted with a microcell counter (Sysmex).

FIG. 48 shows the numbers of MCS-2 cells in the presence of estrone sulfate and testosterone, when an aromatase inhibitor (vorozole), a steroid-sulfatase inhibitor (Compound 17A), or both vorozole and Compound 17A are added (N=3 each).

When Compound 17A is not added, inhibition of the cell proliferation is very low regardless of an increase in the concentration of vorozole. That is, the inhibition of the proliferation is able to be observed at a high concentration of 100 nmol/L. On the contrary, when Compound 17A is added, significant inhibition of the cell proliferation is observed.

The therapeutic agents and pharmaceutical compositions for the treatment of hormone-dependent cancers according to the present invention, which are prepared so as to contain active ingredients from both steroid-sulfatase inhibitors and agents for hormone therapy and/or agents for chemotherapy, can be used, administered, or manufactured in the form of a single preparation or a combination of some preparations. These therapeutic agents in unit dose form are preferable for oral or parenteral (e.g. injection) administration. When the therapeutic agents are used or administered in combination, they may be used or administered together or separately at an interval.

These preparations may contain a pharmaceutically acceptable diluent, excipient, disintegrant, lubricant, binder, surfactant, water, saline, vegetable-oil solubilizer, isotonic agent, preservative, or antioxidant in addition to the effective ingredients, and can be manufactured by a conventional process.

In the preparation of tablets, an excipient, e.g. lactose, a disintegrant, e.g. starch, a lubricant, e.g. magnesium stearate, a binder, e.g. hydroxypropyl cellulose, a surfactant, e.g. fatty acid ester, a plasticizer, e.g. glycerin, and the like may be used according to a conventional process.

In the preparation of injections, water, saline, a vegetable-oil, a solvent, a solubilizer, an isotonic agent, a preservative, an antioxidant, and the like may be used according to a conventional process.

When compound (I), (IA), (IB), and pharmaceutically acceptable salts thereof are used for the above-mentioned purposes, they may be administered orally or parenterally such as injections. An effective dose and frequency of administration depend on the administration form and subject's age, weight, and symptoms. In general, 0.01 to 20 mg/kg/day is preferably administered.

FIG. 43 shows the results of an MTT assay showing the inhibition of MCS-2 cell proliferation when the concentrations of ICI-182780 are constant in the range from 0 to 1.801 nmol/L and Compound 17A is sequentially diluted (1.5-fold for each dilution) from 3.000 nmol/L to 0.004 nmol/L (N=3 each). The vertical axis of the graph represents a relative value of the number of MCS-2 cells in each condition to that in the agent-free condition, which is shown as a relative value (%) of each absorbance. On the horizontal axis of the graph, the amounts of Compound 17A (nmol/L) are shown. Plots on the graph represent concentrations (nmol/L) of ICI-182780.
-∘-: ICI-182780 free
-●-: ICI-182780 at a concentration of 0.030 nmol/L
-⋄-: ICI-182780 at a concentration of 0.051 nmol/L
-♦-: ICI-182780 at a concentration of 0.084 nmol/L
--∘--: ICI-182780 at a concentration of 0.140 nmol/L
-●-: ICI-182780 at a concentration of 0.234 nmol/L
-□-: ICI-182780 at a concentration of 0.390 nmol/L
--□--: ICI-182780 at a concentration of 0.649 nmol/L
-Δ-: ICI-182780 at a concentration of 1.081 nmol/L
-▴-: ICI-182780 at a concentration of 1.801 nmol/L

FIG. 44 shows the results of an MTT assay showing the inhibition of MCS-2 cell proliferation when the concentrations of Compound 17A are constant in the range from 0 to 1.081 nmol/L and ICI-185780 is sequentially diluted (1.5-fold for each dilution) from 10.000 nmol/L to 0.014 nmol/L (N=3 each). The vertical axis of the graph represents a relative value of the number of MCS-2 cells in each condition to that in the agent-free condition, which is shown as a relative value (%) of each absorbance. On the horizontal axis of the graph, the amounts of ICI-182780 (nmol/L) are shown. Plots on the graph represent concentrations (nmol/L) of Compound 17A shown below.
-∘-: Compound 17A free
-●-: Compound 17A at a concentration of 0.018 nmol/L
-⋄-: Compound 17A at a concentration of 0.030 nmol/L
-♦-: Compound 17A at a concentration of 0.051 nmol/L
--∘--: Compound 17A at a concentration of 0.084 nmol/L
-●-: Compound 17A at a concentration of 0.140 nmol/L
-□-: Compound 17A at a concentration of 0.234 nmol/L
--□--: Compound 17A at a concentration of 0.389 nmol/L
-Δ-: Compound 17A at a concentration of 0.649 nmol/L
-▴-: Compound 17A at a concentration of 1.081 nmol/L

FIG. 45 shows an isobologram for the IC50 value constructed from an IC50 value, IC45 value, IC40 value, IC35 value, IC30 value, IC25 value, IC20 value, IC15 value, IC10 value, and IC5 value calculated from the inhibition curves on the MCS-2 cell proliferation shown in FIGS. 43 and 44 for both ICI-182780 alone and Compound 17A alone. The vertical axis of the graph represents a fraction of IC50 of ICI-182780, and the horizontal axis represents that of Compound 17A .

FIG. 46 shows the concentrations (IC50 values) calculated from the inhibition curves shown in FIG. 43 when Compound 17A inhibits 50% of the proliferation of MCS-2 cells under the conditions where the concentrations of ICI-182780 are constant in the range from 0.030 nmol/L to 1.801 nmol/L and Compound 17A is sequentially diluted (1.5-fold for each dilution) from 3.000 nmol/L to 0.004 nmol/L. The concentrations of Compound 17A are plotted with symbol ∘ on the isobologram (FIG. 45).

FIG. 47 shows the concentrations (IC50 values) calculated from the inhibition curves shown in FIG. 44 when ICI-182780 inhibits 50% of the proliferation of MCS-2 cells under the conditions where the concentrations of Compound 17A are constant in the range from 0.018 nmol/L to 1.081 nmol/L and ICI-182780 is sequentially diluted (1.5-fold for each dilution) from 10.000 nmol/L to 0.014 nmol/L. The concentrations of ICI-182780 are plotted with symbol ∘ on the isobologram (FIG. 45).

FIG. 48 shows the inhibition of MCS-2 cell proliferation when a combination of vorozole and Compound 17A is used in the presence of estrone sulfate and testosterone. The vertical axis of the graph represents the number of MCS-2 cells (×10³ cells/mL), and the horizontal axis represents control and vorozole concentration (nmol/L). The three bars show the results when Compound 17A is added at a concentration of, from the left, 0 nmol/L, 3.0 nmol/L, and 10.0 nmol/L.

The formulation examples will be illustrated below, however these examples are never intended to limit the scope of the present invention.

### FORMULATION EXAMPLE 19

(Tablet) (With reference made to Formulation Example 1 of EP 1 568 381, which publication is not admitted to be prior art to the present application)

Tablets having the following composition are prepared according to a conventional procedure.

| | |
|---|---|
| Compound 17A | 5 mg |
| Lactose | 60 mg |
| Potato starch | 30 mg |
| Polyvinylalcohol | 2 mg |
| Magnesium stearate | 1 mg |
| Tar pigment | small amount |

### FORMULATION EXAMPLE 20

(Tablet) (With reference made to Formulation Example 2 of EP 1 568 381, which publication is not admitted to be prior art to the present application)

Tablets having the following composition are prepared according to a conventional procedure.

| | |
|---|---|
| Compound 17A | 5 mg |
| Tamoxifen | 10 mg |
| Lactose | 60 mg |
| Potato starch | 30 mg |
| Polyvinylalcohol | 2 mg |
| Magnesium stearate | 1 mg |
| Tar pigment | small amount |

### PREPARATION EXAMPLE 21

(Injection) (With reference made to Formulation Example 3 of EP 1 568 381, which publication is not admitted to be prior art to the present application)

An injection having the following composition is prepared according to a conventional procedure.

| | |
|---|---|
| Compound 17A | 2 mg |
| D-mannitol | 10 mg |
| Hydrocholoric acid solution | proper amount |
| Sodium hydroxide solution | proper amount |
| Injectable distilled water | proper amount |

### INDUSTRIAL APPLICABILITY

According to the present invention, a therapeutic agent for a hormone-dependent cancer, which comprises (a) a steroid-sulfatase inhibitor and (b) an agent for hormone therapy and/or an agent for chemotherapy, and the like are provided. The above agent shows more excellent activity in treating a hormone-dependent cancer than a steroid-sulfatase alone or an agent for hormone therapy and/or an agent for chemotherapy alone.

1. A method of inhibiting steroid sulphatase activity in a subject in need of same, the method comprising administering to said subject a steroid sulphatase inhibiting amount of a ring system compound;
wherein the ring system compound comprises a ring to which is attached a sulphamate group of the formula 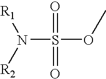
wherein each of R₁ and R₂ is independently selected from H, alkyl, alkenyl, cycloalkyl and aryl, or together represent alkylene optionally containing one or more hetero atoms or groups in the alkylene chain; and
wherein said compound is an inhibitor of an enzyme having steroid sulphatase activity (E.C.3.1.6.2); and
wherein if the sulphamate group of said compound is replaced with a sulphate group to form a sulphate compound and incubated with a steroid sulphatase enzyme (E.C.3.1.6.2) at a pH 7.4 and 37° C. it would provide a Kₘ value of less than 50 μM.

2. A ring system compound;
wherein the ring system compound comprises a ring to which is attached a sulphamate group of the formula 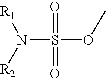
wherein each of R₁ and R₂ is independently selected from H, alkyl, alkenyl, cycloalkyl and aryl, or together represent alkylene optionally containing one or more hetero atoms or groups in the alkylene chain;
wherein R₁ or R₂ is H;
wherein said compound is an inhibitor of an enzyme having steroid sulphatase activity (E.C.3.1.6.2); and
wherein if the sulphamate group of said compound is replaced with a sulphate group to form a sulphate compound it would be a substrate for a steroid sulphatase enzyme (E.C.3.1.6.2).

3. A ring system compound;
wherein the ring system compound comprises a ring to which is attached a sulphamate group of the formula 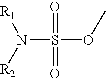
wherein each of R₁ and R₂ is independently selected from H, alkyl, alkenyl, cycloalkyl and aryl, or together represent alkylene optionally containing one or more hetero atoms or groups in the alkylene chain;
wherein R₁ or R₂ is H;
wherein said compound is an inhibitor of an enzyme having steroid sulphatase activity (E.C.3.1.6.2); and
wherein if the sulphamate group of said compound is replaced with a sulphate group to form a sulphate compound and incubated with a steroid sulphatase enzyme E.C.3.1.6.2) at a pH 7.4 and 37° C. it would provide a Kₘ value of less than 50 μM.

4. A ring system compound;
wherein the ring system compound has the formula 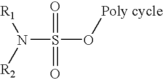
wherein each of R₁ and R₂ is independently selected from H, alkyl, alkenyl, cycloalkyl and aryl, or together represent alkylene optionally containing one or more hetero atoms or groups in the alkylene chain;
wherein R₁ or R₂ is H;
wherein the group Poly cycle is a polycyclic ring structure
wherein said compound is an inhibitor of an enzyme having steroid sulphatase activity (E.C.3.1.6.2); and
wherein if the sulphamate group of said compound is replaced with a sulphate group to form a sulphate compound it would be a substrate for a steroid sulphatase enzyme (E.C.3.1.6.2).

5. A ring system compound;
wherein the ring system compound has the formula 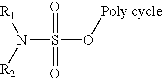
wherein each of R₁ and R₂ is independently selected from H, alkyl, alkenyl, cycloalkyl and aryl, or together represent alkylene optionally containing one or more hetero atoms or groups in the alkylene chain;
wherein R₁ or R₂ is H;
wherein the group Poly cycle is a steroidal ring structure
wherein said compound is an inhibitor of an enzyme having steroid sulphatase activity (E.C.3.1.6.2); and
wherein if the sulphamate group of said compound is replaced with a sulphate group to form a sulphate compound it would be a substrate for a steroid sulphatase enzyme (E.C.3.1.6.2).
